(19) United States
(12) Patent Application Publication
(10) Pub. No.: US 2019/0175515 A1
(43) Pub. Date: Jun. 13, 2019

(54) CHOLESTERYL ESTER VESICLES LOADING PEPTIDES, PROTEINS AND NUCLEIC ACIDS INTO CHYLOMICRONS AND BODY CELLS

(71) Applicants: Jerome J. Schentag, Amherst, NY (US); Mary P. McCourt, Amherst, NY (US); Lawrence Mielnicki, Buffalo, NY (US)

(72) Inventors: Jerome J. Schentag, Amherst, NY (US); Mary P. McCourt, Amherst, NY (US); Lawrence Mielnicki, Buffalo, NY (US)

(21) Appl. No.: 16/327,561

(22) PCT Filed: Aug. 23, 2017
(86) PCT No.: PCT/US2017/048135

§ 371 Date: Feb. 22, 2019

Related U.S. Application Data
(60) Provisional application No. 62/378,599, filed on Aug. 23, 2016.

(51) Int. Cl. A61K 9/48; A61K 9/50; A61K 9/51; A61K 45/06; A61P 35/00

(52) U.S. Cl. CPC A61K 9/4891; A61K 9/4858; A61K 9/5015; A61K 9/0053; A61K 45/06; A61P 35/00; A61K 9/5123

(57) ABSTRACT

The present invention is directed to one or more macromolecules in a lipid vesicle oral formulation which targets intracellular receptors, in particular for peptides, proteins, nucleic acids and mixtures thereof, optionally in combination with small molecules. The invention encapsulates said macromolecules in a neutral lipid vesicle comprised of one or more cholesteryl esters. Unique properties of macromolecules encapsulated in said vesicles include high oral bioavailability, defined herein as in at least 50%, i.e., often in excess of 50% on the basis of oral to parenteral AUC. Non-limiting examples are provided, for large hydrophilic molecules such as peptides, proteins and nucleic acids which heretofore have been very poorly absorbed by the mammalian intestine. In prior art; said molecules are generally less than 25% bioavailable, even with protective coatings and optionally absorption enhancing component substances in the formulation. An additional feature of the present invention is high tissue concentrations after oral use, a result of rapid uptake of cholestosomes delivered by chylomicrons to body cells. A preferred embodiment is disclosed for insulin, where with cholestosome encapsulation oral bioavailability is at least 66%. Prior to the present invention, oral bioavailability of insulin and other peptides and proteins was maximally 25% and usually between 5% and 10%. Additional preferred examples are provided for one or more macromolecules useful in the treatment of cancer and in particular intracellular targeting in the practice of cancer immunotherapeutics.

### RELATED APPLICATIONS

This application claims the benefit of priority of U.S. provisional application Ser. No. 62/378,599, filed Aug. 23, 2016, entitled “Chylomicrons as Carriers for Cholesteryl Ester Vesicles Loaded with Peptides and Proteins for Oral Absorption and Intracellular Delivery”, the entire contents of which is incorporated by reference herein.

### FIELD OF THE INVENTION

The present invention is directed to a means of encapsulating macromolecules, in particular certain biologically active peptides, proteins, nucleic acids and mixtures thereof, and is the first means that accomplishes both oral absorption and intracellular delivery for these large molecules. The invention disclosed herein is a Cholestosome™, which is a neutral charged lipid vesicle with high payload capacity in its center for hydrophilic macromolecules. Cholestosomes containing macromolecules and comprised of one or more cholesteryl esters demonstrate three unique properties over conventional delivery technologies. The first is high oral bioavailability, defined herein as in at least 50%, i.e., often in excess of 50% on the basis of oral to parenteral AUC. Non-limiting examples are provided, for large hydrophilic molecules such as peptides, proteins, nucleic acids and a fluorescent plasmid, all of which heretofore have been very poorly absorbed by the mammalian intestine. The second aspect is loading of the intact vesicle and its macromolecular contents into chylomicrons, a feature unique to absorption of the vesicle by intestinal enterocytes, followed by transfer of the intact vesicle directly into chylomicrons. The third aspect is receptor mediated loading of intact vesicles and their macromolecular contents by body cells, that are docking with cells.

In additional aspects of the present invention, the targeting of immune activation pathways in the distal intestine is now feasible. The invention provides for both distal intestinal delivery and uptake by dendritic cells, the sum of these factors permitting the oral activation of the immune system against cancer in situ. The invention of a practical means of delivering nucleic acids, peptides and proteins inside dendritic cells of the immune system immediately enables a unique means of programming both the innate and adaptive immune responses in a manner never considered possible with earlier technologies, since for the first time, the programming steps can be performed in vivo instead of ex-vivo,

### BACKGROUND AND OVERVIEW OF THE INVENTION

There are many new therapeutic products where a large peptide, protein or other macromolecule is serving a role as a therapeutic or diagnostic substance. Most of these are thought to have their primary action on cell surface receptors, which in turn effect an intracellular sequence of signaling pathways to control synthesis and degradation of molecules in the body. Most of these molecules are too large to be absorbed intact by the cells of the GI tract, and they are degraded in the conditions of the stomach into component amino acids. Overall, these molecules are not absorbed orally, so in virtually all cases they are injected into the body in order to exert their therapeutic actions.

For treatment of chronic conditions, there is a high interest in delivery of large molecules via non-intravenous (IV) routes such as subcutaneous (SC) injection, in order to improve patient convenience and compliance. The usual administration route, parenteral administration, is on the other hand suboptimal for macromolecular delivery for many reasons. Compared to oral administration, parenteral delivery is more expensive and requires hardware and more highly trained personnel. Subcutaneous injection does have advantages over IV use, including that it may be performed by the patient at home. However, oral administration of peptides (including polypeptides such as monoclonal antibodies), proteins, and DNA would be much more convenient and would provide greater safety, so long as oral delivery could be accomplished without damage to the GI tract or from novel materials that create systemic side effects or complications from delivery materials themselves,

Skilled artisans generally believe it is not possible to achieve reasonable bioavailability (greater than 50% of the blood level Area Under the Curve of a subcutaneous injection) via oral administration of large protein molecules in mammals. When given by the oral route, these proteins are not absorbed intact by intestinal cells. Rather, they are broken down by enzymes in the lumen of the intestine into component amino acid constituents, and the components are absorbed by the enterocytes, in such an unprotected formulation, oral formulations are not bio available as, a consequence of degradation by acids, proteases or bile in the stomach and duodenum of the anterior digestive tract. They become inactive. This is particularly true for pharmaceutical compounds such as peptides, proteins, certain small molecules, and nucleic acids. Essentially all of the therapeutic proteins produced by the biotechnology industry are completely susceptible to these gastrointestinal degradation pathways, and they have no chance at reasonable bioavailability.

The skilled artisans working on the degradation problem have tested a variety of protection strategies in order to work around local degradation pathways and improve oral bioavailability of macromolecules. Enteric coating has been employed since the 1960s to bypass the acid barrier in the stomach, where acids activate peptidases and degrade proteins and peptides so that component amino acids can be quickly absorbed. Enteric coating protects against acid degradation in the stomach but the molecule remains susceptible to rapid degradation by the enzymes and bile acids in the duodenum, Enteric coating does not prevent the enterocytes from absorbing and degrading the peptide or protein. Thus, the typical bioavailability achieved by enteric coating a peptide or a protein is around 5% or even substantially less.

Proteinaceous coatings have been employed for many years as a protective coating, typically with an absorption enhancer Sodium N-[8-(2-hydroxybenzoyl)amino]caprylate (SNAC). Examples of this composition are disclosed on the website of Emisphere (www.emisphere.com) and in a press release of Aug. 26, 2015, describing the use of their Eligen® technologies (proteinaceous coating and absorption enhancer SNAC) applied to semaglutide, a GLP-1 agonist under development by Novo Nordisk. Typical oral bioavailability is as low as 1% and as high as 2.5%, according to a disclosure of Novo Nordisk, where a 1.0 mg SC dose once weekly matched the HbA1c lowering of a 40 mg dose orally given once daily Clintrials.gov NCT 01923181. Of note, the Emisphere technology is currently disclosed for oral use with semaglutide in phase 3 trials, as of October 2015.

In some cases, skilled artisans, such as Kidron 2013(1) have successfully enterically coated important peptides such as insulin and GLP-1 and have further combined a protease inhibitor with the formulation to inhibit local enzyme degradation, and have further combined SNAC or Sodium N-[10-(2 hydroxybenzoyl)amino]decanoate (SNAD) as a means of spreading the gaps between cells to force macromolecules into portal blood bypassing the enterocytes themselves. Even with all three of these components of a formulation fully deployed and locally operational, the best oral bioavailability achieved is 10-20% with these strategies. The remaining and still unsolved problem is that intact insulin or GLP-1 is not taken into enterocytes and transferred to blood in the intact and active form. Even when enterocytes internalize an intact peptide, it is likely that the enterocytes themselves further degrade the unprotected insulin or GLP-1 or protein or nucleic material that is taken into the enterocytes. So in a worst case analysis, the absorption enhancer that takes peptides around enterocytes is the most successful component of;the strategy employed by Kidron or Emisphere.

In a similar manner to Kidron, others have tried to make protective outer coatings or particles such as liposomes, which are typically comprised of phospholipids or mixtures of phospholipids and macromolecules such as polyethylene glycols, for example, see Irvine 2011(2). As explained in the following, excerpt from United States Patent Application Document No. 2011-0229529 by Irvine(2), liposomes have not solved the aforementioned problems. Liposomes have been widely used as a delivery vehicle for small molecules; however, it remains difficult to achieve high levels of encapsulation for many macromolecular drugs within liposomes, as these larger molecules tend to be water soluble and thus incompatible with the lipophilic inner core of a typical liposome. While drug delivery by micro- and nanoparticles can encapsulate proteins and small-molecule drugs, this still typically yields very low total mass encapsulated drug per mass of particles, typically on the order of about 1:1000 to 1:10,000 mass ratio, of in this case protein phospholipid mixture (see for example Balu-lyer U.S. Pat. No. 7,662,405) (3).

Liposomes rarely load as high as 1% weightweight, even when using a lipophilic molecule such;as doxorubiein. Cholestosomes as developed by the inventors often will load at least 20% and in non limiting examples presented such as insulin, above 60%, (weight:weight as otherwise described herein),

Furthermore, many drug formulations leak from liposomes too quickly to maintain useful drug delivery kinetics, in addition, the organic solvents used in polymer particle synthesis and hydrophobiclacidic environment within these particles can lead to destruction of therapeutics. (See Zhu et al. Nat Biotechnol. 2000 18:52-57.) (4)

There are other problems with use of liposomes even beyond the aforementioned small amount of encapsulation of water soluble proteins or small molecules. Specifically, the contents of most liposomes are phospholipids, typically phosphatidylcholine, which are amphipathic. These nano sized lipid particles have highly positively charged surfaces and have thereby repelled by the outer membranes of enterocytes and also by cell membranes of peripheral cells.

Phospholipid based liposomes are thus not orally absorbed and are also not able to pass their contents into cells when injected parenterally. Thus, no liposome of current composition is suitable for encapsulation of proteins or peptides (including polypeptides such as monoclonal antibodies). Even if one could somehow load enough protein or peptide into these particles, they would not solve the oral absorption problem, as no phospholipid based liposome can be incorporated into a chylomicron with its molecular payload intact.

Tseng and colleagues further analyzed these problems in 2007(5) and therein tested the hypothesis that adding cholesterol to Phosphatidylcholine liposomes would alter their properties and improve loading. They found only a modest improvement in loading. There was not sufficient cholesterol to change the positive charge of the outer surface. Of greater significance to them was their observation that increased cholesterol in the liposome prevented exit of the loaded molecules, “An increase of the cholesterol content in liposomes results in a dramatic decrease in membrane permeability for non-electrolyte and electrolyte solutes. Optimized drug delivery via liposomes requires the liposome carrier to ultimately become permeable and release the encapsulated drug on the targeted area, but it also requires high stability in the bloodstream” Thus entire the liposomal field largely abandoned cholesterol as a component of liposomes, citing a deterioration in the molecular release properties of cholesterol containing liposomes and further teaching the entire field away from the particular cholesteryl ester vesicles of the present invention.

The particular liposome of Irvine, when given by injection to a mammal, does increase intracellular delivery of macromolecules, and it has been used successfully for transfection of nucleic materials in cells. However, phospholipid compositions in liposomes are particularly unstable in stomach acid and are degraded by intestinal bile acids, which results in release of the peptides and proteins in the intestine where they are accessible to degradation. Accordingly, there is almost never any improvement in oral bioavailability from use of a macromolecule in a phospholipid based, liposome. They are widely used in medicine as controlled release formulations when given by injection. Liposome manufacturing technology generally relies on phospholipid composition and cationic particles are most commonly produced, thereby teaching away from the inventor's use of cholesteryl esters in vesicles with neutral surfaces. In fact, if vesicles are made of phospholipids in the manner of Liposomes disclosed in the art, every one of the uniquely beneficial aspects of the present invention (oral absorption with bioavailability above 50%, intact incorporation into chylomicrons, intracellular delivery of intact payload without reliance on endosomes) is lost, because if they are taken up by cells, an endosomal step is the result.

Prior attempts to deliver macromolecules for oral absorption by the enterocytes have relied on encapsulation in nano sized particles. Most of the work has been conducted with liposomes of varying composition. Recognizing the disadvantages of liposomes, others have produced synthetic nanoparticles that are lipid or polymer based with the same goal of protecting proteins and peptides from degradation in the gastrointestinal tract. For example, Geho 2013(6). Some formulations have included protease inhibitors along with peptides, in an effort to increase the amount of peptide absorbed. The improvement is modest, taking oral bioavailability from perhaps 5% to perhaps 10%, but still in a non-viable range for a strategy to increase bioavailability. The issue is how much more must be provided in order to achieve the same blood levels as would come from injection,

Geho uses a molecular chaperone technology to carry encapsulated molecules across the GI tract by going between the gaps between said cells. These technologies rely on creating local gaps between cells and forcing molecules between said gaps. The methods modestly improve oral bioavailability (typically up to 15-20%) but they also carry the risk of local injury to the enterocytes and other essential cells of the duodenum. Aspects of Geho are relied upon by Kidron and others, typically protease inhibitors and a means of widening the gaps between cells using chemical means such as SNAC or SNAD. The improvement is modest, taking oral bioavailability from perhaps 5% to perhaps 10%, but still in a non-viable range for a strategy to increase bioavailability. The issue is how much more must be provided in order to achieve the same blood levels as would come from injection.

In a combination approach to the challenge of improving oral Insulin bioavailability, Sonaje and colleagues have achieved insulin bioavailability around 17-20% in rats, representing the best combination of the approaches to date.(7-9). They begin with the premise that oral administration of exogenous insulin would deliver the drug directly into the liver through portal circulation, mimicking the physiological fate of endogenously secreted insulin. This characteristic may offer the needed hepatic activation, while avoiding hyperinsulinemia and its associated long-term complications. Kidron and Geho and to our knowledge all other skilled artisans working on insulin and GLP-1 accept this premise, and thus focus on protecting insulin to move it around cells rather than into cells where it would be degraded. Thus, the current standard for improving the oral bioavailability is to protect from degradation and attempt to move the molecule around enterocytes where it can be taken into portal blood. (1, 6). Sonaje uses chitosan nanoparticles (NP)s, arguing that this carrier protects against a harmful gastric environment, and thereby averts enzymatic degradation. Their recent study described a pH-responsive NP system composed of chitosan (CS) and poly (gamma-glutamic acid) for oral delivery of insulin. (7) Chitosan is a nontoxic, soft-tissue compatible, cationic polysaccharide which adheres to the mucosal surface and transiently opens the tight junctions (TJs) between contiguous epithelial cells. Therefore, drugs made with CS NPs would have delivery advantages over traditional tablet or powder formulations. These CS NPs can adhere to and infiltrate the mucus layer in the small intestine. Subsequently, the infiltrated CS NPs transiently open the TJs between epithelial cells. Because they are pH-sensitive, the nanoparticles become less stable and disintegrate, releasing the loaded insulin. The insulin then permeates through the opened paracellular pathway, bypassing absorption by duodenal emerocytes and moves into the systemic circulation. Calculated bioavailability of Aspart Insulin in this rat model was 17%.

Later work by these skilled artisans retained the CS coating but changed the absorption enhancer from poly (gamma-glutamic acid) to add DTPA, forming, gamma poly glutamic acid—Diethylene triamine pentaacetic acid, known as gammaPGA-DTPA. Experimental results indicate that CS/gammaPGA-DTPA NPs can promote the insulin absorption throughout the entire small intestine of rats; the absorbed insulin was clearly identified in the kidney and bladder. In addition to producing a prolonged reduction in blood glucose levels, the oral intake of the enteric-coated capsule containing CS/gammaPGA-DTPA NPs showed a maximum insulin concentration at 4 h after treatment. The relative oral bioavailability of insulin was approximately 20%(8). These studies do move the bioavailability from 5-10% to approaching the theoretical limit of 25%. The protease inhibitor is clearly important but insufficient, so it remains necessary to further improve the poor bioavailability of proteins with a novel means of taking up proteins into enterocytes while preventing further molecular catabolism thereby. The problem remains that free insulin taken into enterocytes is degraded in those enterocytes Depending on the exact site of intestinal release, the strategies that move insulin around enterocytes into portal blood still suffer from the lack of uptake by the enterocytes without catabolism, and that is the primary means for degradation of insulin when given orally.

Regarding the strategy of passing the cell membrane with a coating, there have been nanoparticles that move materials inside cells. Hong 2004 described a micelle like nanoparticle delivery means in US 2004-0037874A1(10). This cationic nanoparticle was primarily used to encapsulate the anionic nucleic acids which may be useful for transfection of genetic materials into cells. It was likely subjected to endosomal uptake during passage of the cell membrane, and its contents were thus degraded. Furthermore, it was not suitable for oral use however, because it was not stable in the harsh environment of the GI tract and it would be unlikely to be taken up intact by enterocytes.

Even after parenteral administration, most macromolecules encounter problems with passage of membranes, and most delivery systems fail to provide intact macromolecules inside cells. They are excluded from many target cells, and as a result they circulate in blood until cleared or degraded but may never successfully enter body cells. Endosomal uptake may destroy them and they may never reach their intracellular targets in an active form Macromolecules either alone or in delivery means may fail to pass regional barriers such as the blood brain barrier, effectively preventing targeting of macromolecules to selected organs and tissues such as brain. Failure to reach into target cells may be an underlying reason for clinical trial failure of many of the monoclonal antibodies against targets in the amyloid pathway to clear amyloid from the brain and their lack of sufficient activity to reverse Alzheimer's disease. In general, the large size and lack of lipid solubility of these proteins may limit the intracellular effectiveness of an otherwise novel target monoclonal antibody.

Nagy in 2014 demonstrated a sophisticated intracellular delivery system, also applied to nucleic acids, where release inside the cell depended on enzymatic degradation of the particle itself (11). However, this particle as well does not appear stable in the GI tract and it would not be placed intact into a chylomicron fur delivery to lymphatics and then to body cells. The Nagy particle was also likely degraded in the endosomes of the cells that take it up.

All of these technologies are up against the primary limitation of this and all other lipid based particles, which is the inability to be taken up intact by enterocytes and remain intact during passage thru the enterocyte into blood on the basolateral side of the intestinal barrier.

Because orally administered molecules such as proteins, peptides and genetic material are either digested in the gastrointestinal (GI) tract or fail to diffuse in an intact form across the cellular membrane of the enterocytes, or both, skilled artisans widely believe that oral bioavailability barrier remains at 25% or less absorption thru the GI tract, and thus for proteins, peptides, and nucleic acids, IV or SC administration is the only reliable way to administer such active pharmaceutical agents.

A fundamental challenge plaguing oral delivery of peptides using current technology is the quantity of medication that must be orally administered to effect the desired outcome in a patient. For example, if the molecule is 2.5% bioavailable as with the semaglutide example, then the oral dose is 40 times the injected dose, and this is a cost of goods consequence that probably matters for production of the molecule itself Poor bioavailability due to a bad solubility profile or degradation of the surface coating can mean that even though a certain medication tolerates the digestive milieu, it cannot be given orally in any meaningful way. It may, for example, need to be given in substantially larger doses than would be required if given intravenously, or via another injectable route of administration.

It is a sign of these desperate times that one major company has placed the aforementioned oral formulation of oral semaglutide into phase 3 trials, even though the dose is 10-40 times greater orally than subcutaneously. The additional problem besides the obvious cost of goods increase is the potential for great variability in absorption, with resultant toxic side effects if more than usual is absorbed due to unknown factors on a particular day, such as a food effect.

The inventors disclose a new approach to the enterocytes and their associated degradation of orally administered proteins and peptides. The invention is simple in concept, uses non-toxic formulation materials and has unexpectedly yielded near 100% bioavailability in mice and rats. The resultant delivery vesicle is stable in the GI tract and completely absorbed by the enterocytes. However, the enterocytes do not degrade the particle and instead insert the particle, and its contents into chylomicrons for delivery to body cells via lymphatics. The delivery vesicle thus protects its contents dim the GI tract and thin the cell membranes of body cells, delivering for the first time, an intact payload inside the targeted cells. Any cell that expresses a surface receptor for chylomicron docking, enabling the intracellular delivery of the vesicle and its contents, is a targeted cell for this novel delivery means. The features of the invention have been disclosed by McCourt(12) and Schentag and McCourt(13). The unexpected ability to overcome the 25% bioavailability barrier is the subject of the present invention, disclosed herein.

Clearly, success with oral proteins depends on creation of novel formulations that overcome acid and/or enzymatic degradation in the GI tract and then overcome low permeability across an intestinal enterocyte membrane, and finally a delivery method must overcome the current inability to pass peptides into the cells and release them from the delivery means intact on the other side of the cell membranes. All of the aforementioned limitations and disadvantages have been remedied for insulin, by way of example, as will be subsequently disclosed herein.

Insulin is a medicament used to treat patients suffering from diabetes, and is the only treatment for insulin-dependent diabetes mellitus. Diabetes Mellitus is characterized by a pathological condition of absolute or relative insulin deficiency, leading to hyperglycemia, and is one of the main threats to human health in the 21st century. The global burden of people with diabetes is set at 220 million in 2010, and 330 million in 2025. Type I diabetes is caused primarily by the failure of the pancreas to produce insulin. Type II diabetes, involves a lack of responsiveness of the body to the action of insulin, a state which is termed insulin resistance. Insulin resistance is a precursor to many other metabolic diseases, such as obesity, Hepatic steatosis, atherosclerotic heart diseases, and even many forms of cancer.

Approximately 20%-30% of all diabetics use daily insulin injections to maintain their glucose levels. An estimated 10% of all diabetics are totally dependent on insulin injections.

Currently, the only route of insulin administration is injection. Daily injection of insulin causes considerable suffering for patients and can impact patient compliance. The greatest problem is hypoglycemia, which can be severe and in some cases life threatening. Side effects such as lipodystrophy, lipoatrophy, and lipohypertrophy, occur at the site of injection. In addition, subcutaneous injection of insulin does not typically provide the fine continuous retaliation of metabolism that occurs normally with insulin secreted from the pancreas directly into the liver via the portal vein.

The present invention addresses the need for an alternate solution for administration of insulin and peptides such as insulin. For the first time the present invention provides for an oral dosage of insulin that is the same as the injectable dose, which would be the expected outcome of a delivery means that achieves 100% oral bioavailability,

Recent oral insulin and other peptide formulations need a protease inhibitor and an absorption enhancer. (1) Experimental data show that protease inhibitors may partially overcome the gastrointestinal degradation problems with oral insulin formulations. (1) However, even where there is improvement in oral absorption, the maximum effect converts no absorption at all to perhaps 10-15% absorption. Even with these enhancements, the oral bioavailability remains low and variable and as a result the oral dose is often 10 times greater than would be given by injection, which exposes the patient to potentially lethal amounts of insulin should the entire administered dose actually be absorbed on any given day.

Of great importance, the delivery means of the present invention is the first to solve the next problem, that of intracellular delivery to enterocytes without enterocyte metabolism, by means of a transformative step performed on the vesicle, the incorporation of the lipid vesicle into chylomicrons with its molecular payload intact. Successful incorporation into chylomicrons is only possible with the use of herein disclosed cholesteryl esters to construct the lipid vesicle. No other biological encapsulation method known will provide for intact uptake of a vesicle by gastrointestinal enterocytes.

It should be noted in the present invention, that the inventors have chosen the high loading (i.e.., cargo is 20% to 96%, 25% to 95%, often 25-90%,10% to 80%, 25% to 75% by weight of the total weight of a cargo-loaded vesicle which comprises active compounds and vesicle, with a preferred vesicle size ranging from 750 nm to 7,500 nm, more often 1,500 to 2,500 nm, more often 2000 nm) and slow release properties with preferred specific mixtures of C₈ to C₁₄ cholesteryl esters for the specific purposes of protecting the molecule during its journey across membranes of the GI tract enterocytes, thus keeping the payload intact while incorporating the entire vesicle into chylomicrons.

Chylomicrons then become the perfect lipid delivery means for lipids such as cholesteryl esters, so there is no resistance by cells to uptake, which occurs normally. There is no endosomal step when cholesteryl ester vesicles and their intact payload pass thru the cell membrane into cytoplasm. Unpacking of cholesteryl ester encapsulated proteins only occurs inside the body cells after delivery to those cells, which confers a great advantage to the disclosed delivery method over any current system. Thus, the present inventors disclose the analogy to the Trojan Horse, invented of course before there were patents, but not used heretofore to synthesize a peptide delivery means that creates a surprise inside the cell when unpacking occurs and the molecule usually excluded from cells, is now inside to effect an intended outcome.

It should also be noted that the disclosed process works as disclosed only with cholesteryl esters, as only these molecules are handled intact among lipids all the way to intracellular delivery by chylomicrons and unpacking inside cells.

Given the limitations of existing macromolecule therapies, the need continues to exist for formulations and treatments that administer pharmaceutically active macromolecules in a more convenient way such as orally, and the need continues for formulations that allow proteins and other molecules to enter cells. The use of one formulation means to, accomplish both aspects (oral absorption and intracellular delivery) is but one unique aspect of the present invention.

It should be noted that in the present invention, the inventors preferably have chosen the high loading and slow release properties specific mixtures of C₅ to C₁₄ cholesteryl esters for the specific purposes of protecting the molecule during its journey across membranes of the GI tract enterocytes, then keeping the payload intact while incorporating the entire vesicle into chylomicrons.

Chylomicrons then become the perfect lipid delivery means for lipids such as cholesteryl esters, so there is no resistance by cells to uptake, which occurs normally. There is no endosomal step when cholesteryl ester vesicles and their intact payload pass thru the cell membrane into cytoplasm. Unpacking of cholesteryl ester encapsulated proteins only occurs inside the body cells, which confers a great advantage to the disclosed delivery method over any current system.

It should also be noted that the disclosed process works as disclosed only with cholesteryl esters, as only these molecules are handled intact among lipids all the way to intracellular delivery by chylomicrons and unpacking inside cells.

Given the limitations of existing macromolecule therapies, the need continues to exist for formulations and treatments that administer pharmaceutically active macromolecules in a more convenient way such as orally, and the need continues for formulations that allow proteins and other molecules to enter cells. The use of one formulation means to accomplish both aspects (oral absorption and intracellular delivery) is but one unique aspect of the present invention. The present invention also is directed to a means of loading a protein or peptide into cells expressing a receptor for chylomicrons, which contain the molecule within a lipid vesicle. This method is suitable for oral use and when used as an oral delivery means, bioavailability for peptides and proteins is almost 100%, depending on the molecule, and the means of encapsulation. When using the method disclosed herein, cells are loaded with intact molecules and the passage into the cell occurs without forming an endosome. Pursuant to the present invention, the novel cargo loaded cholestosomes according to the present invention are capable of depositing active molecules within cells of a patient or subject and effecting therapy or diagnosis of the patient or subject. This method is also enhanced by delivery of an inhibitor of intracellular metabolism, as the non-metabolized molecules are released from the cells to circulate in bio-fluids until otherwise excreted.

### SUMMARY OF THE INVENTION

The present invention is directed to a means of enclosing a hydrophilic macromolecule inside a lipid, vesicle comprised of one or more cholesteryl esters, and by means of the favorable surface properties of the vesicle, moving the protein or peptide inside the GI tract enterocytes without those cells degrading said protein or said vesicle. As a second step, the enterocyte moves the intact vesicle into chylomicrons for lymphatic transfer into blood. As a third step, the chylomicrons dock with cells (which preferably express chylomicron receptors) and then load these cells with the cholesteryl ester vesicles and their macromolecular payload. The result of this novel invention is both high oral bioavailability and intracellular delivery for said macromolecule. The delivery means disclosed herein surprisingly accomplishes both oral uptake and intracellular delivery, neither of which have been successfully accomplished prior to this invention with macromolcules. The prior art approaches focused on moving these and similar larger molecules around and between enterocytes, but have not succeeded with a means of moving molecules through enterocytes and from there inside cells of the body.

An embodiment of the invention, as described above, is a means of moving the protein or peptide into a chylomicron in the golgi apparatus of GI tract enterocytes, then releasing the loaded chylomicron from the enterocytes for circulation in the lymphatics and blood until delivery of the composition into cells expressing a receptor for attachment of chylomicrons. The method is suitable for oral use and when used as an oral delivery means, bioavailability for peptides and proteins is almost 100%, depending on the molecule, and the means of encapsulation. When using the method disclosed herein, cells are loaded with intact molecules and the passage into the cell occurs surprisingly without forming an endosome. Pursuant to the present invention, the novel cargo loaded cholesteryl ester vesicles prepared according to the present invention are capable of depositing active molecules within cells of a patient or subject and effecting therapy or diagnosis of the patient or subject. In embodiments, this method is also enhanced by delivery of an inhibitor of intracellular metabolism, as the non-metabolized molecules are released from the cells to circulate in bio-fluids until otherwise excreted. Prior art approaches have focused on coatings which do not incorporate into chylomicrons and which do not easily pass the cell membrane without requirement fear an endosome. These prior art methods are less effective at intracellular cell delivery of intact payloads.

This invention provides, by means of fully enabled examples, compositions comprising one or more peptides and optionally one or more inhibitors of intracellular peptide metabolism, for oral use in the treatment of a human patient in need thereof. In preferred embodiments, said compositions are useful in the treatment of cancer, infectious diseases, diabetes, obesity, insulin resistance, fatty liver diseases, non-alcoholic steatohepatitis (NASH), and metabolic syndrome.

In a preferred embodiment, the peptide is an Insulin, optionally in combination with a GLP-1 agonist, but these embodiments are not limiting and any peptide, protein, or other molecule in any amount falls clearly within the scope of the invention. The loaded vesicle may contain one or more peptides as a mixture, and the mixture of peptides may also contain an inhibitor of peptide metabolism for purposes of prolonging the residence time of the molecule once released inside cells.

The steps of the invention require one or more coating materials which consist essentially of C₆ to G₂₆ cholesteryl esters (i.e. the cholesteryl ester is fanned from cholesterol and a C₆-C₂₆ fatty acid forming an ester) and other components which do not materially impact the basic characteristics of the vesicle which provide enhanced delivery of macromolecules to cells, especially, but not exclusively, by oral routes of administration as otherwise described herein), preferably C₆-C₂₂, C₈-C₂₂, C₈-C₁₈, preferably C₈ to C₁₄ cholesteryl esters, which uniquely form a vesicle with a hydrophilic hollow center and a neutral charge on both inner and outer surface of the vesicle. The vesicle, once loaded with a macromolecule such as a peptide (insulin in a preferred embodiment) is uniquely taken into enterocytes by intestinal enterocyte surface transporters located on the apex brush border of the enterocytes. The intact vesicle which contains macromolcule is not altered within the cell after uptake, because the surface of the vesicle is recognized by enterocytes as a nutritional element (both components, fatty acids and cholesterol are needed together as cholesteryl esters). The enterocytes place the vesicles into nascent chylomicrons with internal peptide payload intact and un-recognized. From this step, the chylomicrons are sent to lymph channels by the enterocytes and thereby enabling chylomicron delivery of these cargo-loaded cholesteryl ester (lipid) vesicles into body non-enterocyte cells expressing a surface receptor for said chylomicron.

The cholesteryl esters used in the construction of said cholesteryl ester vesicle are produced from cholesterol (as defined herein) and one or more saturated or unsaturated fatty acids as otherwise described herein. The vesicles disclosed as preferred delivery means in the invention are constructed using at least one non-ionic cholesteryl ester of C₆-C₂₆, C₆-C₂₂, C₈-C₂₂, C₈-C₁₈, preferably C₈ to C₁₄, the optimal embodiment in the composition of a vesicle which is a 40:60 to 60:40, preferably a 55:45 to 45:55 or 50:50 mass ratio of Myristic acid to Laurie acid, which is then optimally recognized by apical surface transporters on enterocytes and taken into these cells as an intact vesicle.

These cholesteryl ester vesicles are cyclized around one or more encapsulated active molecules. Cholesteryl esters made from fatty acids less than C₆ and in particular C₂-C₅, including C₄ do not readily cyclize, making them unsuitable for encapsulation in vesicles according to the present invention. Cholesteryl esters longer than C₁₆ are less optimally internalized into enterocytes by the apical fatty acid transporters. The delivery means is suitable for molecules of varied sizes and all of which, in the absence of encapsulation in cholesteryl esters, cannot appreciably pass through an enterocyte membrane in the absence of said molecule being loaded into said cholesteryl ester vesicle. Specific composition of the vesicle conveys the uptake by enterocytes and ability to pass into enterocytes in the manner of orally absorbed nutrient fatty acids and cholesterol using cell pathways to reach the Golgi apparatus. Pursuant to the present invention, the novel cargo loaded vesicles of the present invention will deposit intact peptides, proteins or nucleic acids molecules within cells of a patient or subject and effecting therapy of the patient or subject.

Intact cholesteryl ester vesicles within the scope of the invention are surprisingly taken into duodenal enterocytes by specific fatty acid transporters. Further surprisingly, the internalized vesicles are then rapidly transferred, with outer membranes remaining intact, along with their intact contents, into chylomicrons in the Golgi apparatus. The chylomicrons loaded with the peptides within cholesteryl ester vesicles enter lymphatics and then the bloodstream via the thoracic duct, thereafter providing a means of transporting the encapsulated peptides directly into body cells that express a surface receptor for the apolipoprotein that is an integral part of the enterocyte loaded chylomicron. The result is unexpectedly high bioavailability on the order of 50% to upwards of 100% as defined herein.

Once inside body cells, cholesteryl ester hydrolase enzymes act on the bond between cholesterol and the fatty acids of the delivery means, the result is a release said peptides and optionally the release of an inhibitor of peptide metabolism within the cells. Body cells optionally incorporate the delivered peptides into the cells, metabolize them and/or eject the peptides out of cells into blood either as free peptides or peptides within cholesteryl ester vesicles. Surprisingly, the inventors have discovered that cells may optionally eject intact cholesteryl ester vesicles to continue their journey around the body, still with payload intact. These may be taken up intact by other cells, including cells which do not express a surface receptor for chylomicrons (apolipoproteins). This unusual recirculating pattern is surprisingly more pronounced in mice as compared to rats and leads to longer retention times in the animal and in some instances, more favorable pharmacokinetics. It is expected that this recirculating pattern will also occur in humans.

Preferred embodiments of the presently claimed peptide-loaded cholesteryl ester vesicles provide high blood levels of insulin and nearly complete oral bioavailability in studies of mice and rats. Cholesteryl ester vesicles selected for C₁₂:C₁₄ cholesteryl esters in a 1:1 molar ratio, and prepared under specific pH and insulin concentrations, deliver a maximal amount of insulin into chylomicrons and thereby into body cells. The method as disclosed herein can be adapted to encapsulate any peptide, including oligo and polypeptides (including polypeptides such as monoclonal antibodies) and proteins and other macromolecules, including polynucleotides such as DNA and RNA, which vary greatly in size and molecular weight, into cells via the oral route. In a disclosed preferred embodiment, the cholesteryl ester vesicles are used to load cells with GFP plasmids, the cells then expressing the characteristic green fluorescence, evidencing that the cholesteryl ester vesicles according to the present invention may readily encapusulate numerous nucleic acids and delivery these macromolecules into cells as otherwise described herein.

Accordingly, in embodiments, the present invention is directed to a pharmaceutical composition in oral dosage form. In one, embodiment, the present invention is directed to a pharmaceutical composition in oral dosage form for administration to a mammal, especially including a human, comprising a vesicle encapsulating a core comprising at least one pharmaceutically active agent which is a macromolecule (e.g., peptide, protein, nucleic acid, other active agent including an antibiotic, antiviral agent, antifungal agent, etc.) preferably an insulin and optionally one or more additional molecules, wherein said vesicle has an outer surface coating consisting essentially of one or more cholesteryl esters obtained from cholesterol and a C₆-C₂₆ fatty acid (preferably a C₈-C₂₂ fatty acid, or a C₈-C₁₄ fatty acid), wherein said outer surface coating: of said vesicle remains intact during passage of said composition across a cell membrane such that said vesicle and its core enters into body cells of said mammal without endosomal formation, the macromolecule and the optional additional molecule(s) comprising 20% to 96%, often 25% to 95% by weight of the total weight of the vesicle, in preferred aspects, the macromolecule is an insulin, and the composition further comprises a GLP-1 agonist and an inhibitor of the degradation of the insulin and/or the GLP-1 agonist.

In alternative embodiments, the present invention is directed to a pharmaceutical composition in oral dosage form comprising at least one pharmaceutically active agent which is an insulin and optionally one or more additional molecules which are all encapsulated within the core of a vesicle, wherein the vesicle has an outer surface which comprises one or more cholesteryl esters obtained from cholesterol and one or more C₆-C₂₆, preferably C₅-C₁₄ saturated or unsaturated fatty acids, wherein said pharmaceutically active agent comprises about 20% to about 96%, about 25% to about 95%, preferably about 25% to about. 80% by weight of said active agent and said vesicle, and the composition produces a bioavailability of at least 50% of active agent after oral administration of the composition to a patient. In an alternative embodment, the active agent is an insulin and the composition further includes a GLP-1 molecule and optionally, an inhibitor of intracellular metabolism of one or both of said insulin and said GLP-1 molecule, in another embodiment, the composition comprises a cholesteryl ester Obtained from cholesteryl ester and a C₈-C₁₄ fatty acid and the composition produces a bioavailability after administration to the patient of 60% to 100%. In still other embodiments, in the composition according to the present invention, the fatty acid is selected from the group consisting Myristoleic acid, Palmitoleic acid, Sapienic acid, Oleic acid, Elaidic acid, Vaccenic acid, Linoleic acid, Linoelaidic acid, α-Linolenic acid, Arachidonic acid, Eicosapentaenoic acid, Erucic acid, Docosahexaenoic acid, Caprylic acid, Capric acid, Laurie acid, Myristic acid, Palmitic acid, Stearic acid, Arachidic acid, Behenic acid, Lignoceric acid, Cerotic acid or a mixture thereof.

In still other embodiments, the present invention is directed to a pharmaceutical composition comprising a poly-neo-epitope mRNA cancer immunotherapy antigen construct and an optional adjuvant encapsulated within the core of a vesicle to produce a cargo-loaded vesicle, wherein said cargo-loaded vesicle has an outer surface which is comprised of one or more cholesteryl esters obtained from cholesterol and one or more C₆-C₂₆, preferably C₅-C₁₄ saturated or unsaturated fatty acids, wherein said antigen construct and said optional adjuvant comprises about 1% to about 96% by weight of said cargo-loaded vesicle, preferably about 1% to about 50%, about 2% to about 25% by weight of said cargo-loaded vesicle. In an alternative embodiment, the composition is in oral dosage form wherein the composition is adapted to deliver the mRNA cancer immunotherapy antigen construct and optional adjuvant to the ileum and/or appendix of a patient or subject. In still other embodiments, the adjuvant is a lipopolysaccharide (LPS) adjuvant. In another embodiment, according to the present invention, the poly-neo-epitope mRNA cancer immunotherapy antigen construct is autologous, meaning that the construct is derived from cancer cells of the patient to be treated. In alternative embodiments, the cancer immunotherapy construct is allogeneic, meaning that the construct is derived from suitable cancer cells not obtained from the patient. Another embodiment is directed to a method of treating cancer in a patient in need comprising administering to said patient an effective amount of the composition as described above, alone or in combination with an adjuvant, and alone or in combination with a checkpoint inhibitor. In still other embodiment of the invention, an entire plasmid is transported into cells using said lipid vesicles. This latter discovery provides a means of transfection of cells with nucleic acids using an oral dosage form. As no viral vector is required, the disclosed transfection means represents a novel and potentially safer means of inserting nucleic acid constructs into cells.

Thus, in one embodiment, the invention is directed to composition in pharmaceutical dosage form for administration to a patient or subject comprising one or more macromolecules encapsulated in a lipid vesicle to provide an intact loaded vesicle wherein the outer surface coating of the loaded vesicle comprises at least one cholesteryl ester obtained from cholesterol and a C₆-C₂₆ fatty acid. (preferably C₆-C₂₂, preferably C₈ to C₁₄ fatty acid), said macromolecule obtaining an intracellular concentration in cells of said patient or subject which is at least 10-fold greater than the concentration obtained by said macromolecule in the absence of said vesicle.

In preferred aspects of the invention, the composition is formulated in oral dosage form for administration to a patient or subject, preferably a human patient.

In an additional embodiment, the invention is directed to a composition including a composition described above wherein the outer surface coating of the loaded vesicle remains intact without endosomal formation during passage of said loaded vesicle across a membrane of a cell in said patient or subject, and wherein said vesicle optionally releases said one or more macromolecules inside the cell by the action of cholesteryl ester hydrolases on said vesicle.

In a further embodiment, the invention is directed to a composition, including a composition described above wherein the macromolecule is selected from the group consisting of proteins, peptides, nucleic acids and mixtures thereof.

In still another embodiment, the invention is directed to a composition, including a composition described above wherein the macromolecule obtains an intracellular concentration at, least 250-fold greater than the concentration obtained by the macromolecule in the absence of the vesicle.

In yet another embodiment, the invention is directed to a composition, including a composition described above wherein the intact vesicle binds to cells of said patient or subject which express surface receptors for the chylomicrons (targeted cells) and the intact vesicle releases the macromolecule(s) in the cell by the action of cholesteryl ester hydrolases in the cells on said vesicle.

In still a further embodiment, the invention is directed to a composition, including a composition described above wherein the intracellular concentration of macromolecule in cells expressing surface chylomicron receptors is at least 10 times greater than the intracellular concentration of macromolecule in cells which do not express surface chylomicron receptors in said patient or subject.

Yet another embodiment of the invention is directed to a composition, including a composition described above wherein the intracellular concentration of macromolecule in cells expressing a surface chylomicron receptor is at least 10 times greater than the intracellular concentration of macromolecule in vesicles which do not form loaded chylomicrons.

In still another embodiment, the invention is directed to a composition, including a composition described above wherein the intracellular concentration of macromolecule in, cells expressing a surface chylomicron receptor is at least 250 times greater than the intracellular concentration of macromolecule in vesicles which do not form loaded chylomicrons.

In another embodiment, the invention is directed to a composition, including a composition described above wherein the intact loaded vesicles enter cells in the patient or subject and the cells use cholesteryl ester hydrolases to release macromolecule from the vesicles, wherein the cells optionally eject a portion of the intact vesicles from the cells into the extracellular fluid surrounding the cells.

In a further embodiment, the invention is directed to a composition, including a composition described above wherein the intact vesicles are opened by cholesteryl ester hydrolase in said cell and said macromolecule acts on components in said cell.

In still a further embodiment, the invention is directed to a composition, including a composition described above wherein the cell metabolizes the macromolecule and/or ejects the macromolecule from the cell.

In still an additional embodiment, the invention is directed to a composition, including a composition described above wherein the macromolecule is unaltered during encapsulation into the intact vesicle and upon release in the cells by cholesteryl ester hydrolase and is identical to and has the same activity as the macromolecule encapsulated in the vesicle.

A further embodiment is directed to a composition, including a composition described above wherein the macromolecule is insulin.

In yet another embodiment, the invention is directed to a composition, including a composition described above is in oral dosage form which is optionally enteric coated, wherein said macromolecule reaches the blood stream of patient or subject at a concentration between at least 50 percent and 100 percent of the area under the curve (AUC) blood concentration when the insulin is administered to the patient or subject by subcutaneous or intravenous injection,

In still a further additional embodiment, the invention is directed to a composition, including, a composition described above which further incorporates a protease inhibitor in combination with the macromolecule in the core of the vesicle.

Another embodiment is directed to a composition, including a composition described above wherein the protease inhibitor inhibits the cell from metabolizing the macromolecule and wherein the cell ejects more of the macromolecule into the extracellular fluid surrounding than would occur in the absence of the protease inhibitor.

In yet another embodiment, the invention is directed to a composition, including a composition described above wherein the vesicle comprises insulin and at least one additional macromolecule.

The invention is also directed to an embodiment wherein the composition, including a composition described above, which further includes an inhibitor of extracellular metabolism of the insulin and/or the additional macromolecule.

Another embodiment is directed to a composition, including a composition described above, in oral dosage form wherein the intact vesicle passes through the intestinal enterocytes and into chylomicrons in the enterocytes and wherein cells in the patient or subject express receptors for the chylomicrons and the cells attain higher intracellular concentrations and release greater amounts of the insulin and the additional macromolecule than cells which lack surface receptors for the chylomicrons.

Another embodiment is directed to a composition, including a composition described above, wherein the additional macromolecule is bacitracin.

In still an additional embodiment, the present invention is directed to a composition, including a composition described above, which further includes an IDE inhibitor, a DPP-IV inhibitor or a mixture thereof.

An addition embodiment of the present invention is directed to a composition, including a composition described above, which further includes a GLP-1 antagonist.

In yet a further additional embodiment, the present invention is directed to a composition, including a composition described above, which comprises two macromolecules in said vesicle core.

In still a further embodiment, the present invention is directed to a composition, including a composition described above, wherein the first macromolecule is insulin, and the second macromolecule is a GLP-1 agonist.

In still another embodiment, the present invention is directed to a composition, including a composition described above, wherein the vesicle optionally includes an inhibitor of cellular metabolism of the macromolecule.

In still an additional embodiment, the present invention is directed to a composition, including a composition described above, wherein the macromolecule is trastuzumab and wherein the trastuzumab is loaded into said vesicles at 40 to 60 percent t by weight of the total weight of the cargo loaded vesicles at a pH of 5.5-6.5 preferably a pH of approximately 6.0.

The present invention also directed to a composition, including a composition as described above, wherein the macromolecule is exenatide and said vesicle further includes a DPP-IV inhibitor.

The present invention is also directed to yet another composition, including a composition as described above, wherein said DPP-IV inhibitor is sitagliptin, saxagliptin, linagliptin or a mixture thereof.

In another embodiment, the present invention is directed to a composition, including a composition described above, wherein the macromolecule is a GLP-1 molecule that has been modified to improve stability to DPP-IV enzymatic, degradation or has been modified to prolong its circulation time in the blood.

In a further embodiment, the present invention is directed to a composition, including a composition described above, wherein the GLP-1 molecule is liraglutide, dulaglutide, semaglutide, Lixisenatide, albigiutide or a derivative thereof, and the composition optionally includes an inhibitor of intracellular metabolism of the GLP-1 molecule.

In still a further embodiment, the present invention is directed to a composition, including a composition described above, wherein the macromolecule is a GLP-1 molecule selected from the group consisting of liraglutide, dulaglutide, semaglutide, lixisenatide, albighnide, or a derivative thereof, and the composition farther includes an insulin selected from the group consisting of recombinant insulin, NPH insulin, Lente insulin, insulin glargine, insulin lispro, novolog, or insulin degludec and the composition optionally comprises an inhibitor of intracellular metabolism of one or both of said GLP-1 molecule and the insulin.

In another embodiment, the present invention is directed to a composition, including a composition described above, wherein the GLP-1 molecule is lixisenatide, the insulin is glargine and the optional inhibitor of intracellular metabolism is sitagliptin.

In yet an additional embodiment, the present invention is directed to a composition, including a composition described above, wherein the Insulin is insulin Lispro and the GLP-1 molecule is dulaglutide, and the optional inhibitor is Linagliptin

The present invention is also directed to a composition, including a composition described above, wherein the insulin is degludec, the GLP-1 molecule is semaglutide, and the optional inhibitor is sitagliptin.

In a further embodiment, the present invention is directed to a composition, including a composition described above, wherein the Insulin is Novolog, the GLP-1 molecule is Liraglutide, and the optional inhibitor is sitagliptin.

In another embodiment, the present invention is directed to a composition, including a composition described above, wherein oral administration of the macromolecule produces bioavailability of the macromolecule in the patient or subject of at least 50%.

In yet an additional embodiment, the present invention is directed to a composition, including a composition described above wherein the bioavailability is 85-100%.

In still another embodiment, the present invention is directed to a composition, including a composition described above, wherein oral administration of the macromolecule produces a tissue concentration at least 10 times greater than the plasma concentration of the macromolecule.

In an alternative embodiment, the present invention is directed to a composition, including a composition described above, wherein oral administration of the macromolecule, produces a tissue concentration at least 20 times greater than the plasma concentration of the macromolecule.

In still another embodiment, the present invention is directed to a composition, including a composition described above, wherein oral administration of the macromolecule produces a tissue concentration up to 250 times the plasma concentration of the macromolecule.

In a further embodiment, the present invention is directed to a composition, including a composition described above, wherein the cholesteryl esters are a mixture of two different cholesteryl esters.

In yet another embodiment, the present invention is directed to a composition, including a composition described above, wherein the cholesteryl ester components are obtained from fatty acids which differ in length by more than two carbon units.

In an additional alternative embodiment, the present invention is directed to a composition, including a composition described above, wherein the cholesteryl ester components are obtained from fatty acids which differ in length by no more than two carbon units.

In a further embodiment, the present invention is directed to a composition, including a composition described above, wherein the cholesteryl ester components are obtained from fatty acids which differ in length by two carbon units.

In yet another embodiment, the present invention is directed to a composition, including a composition described above, wherein the vesicles further comprise an effective amount of phosphatidyl serine to target cells for apoptosis.

In still an alternative embodiment, the present invention is directed to a composition, including a composition described above, wherein the macromolecule is a nucleic acid, preferably including a plasmid.

In yet an additional embodiment, the present invention is directed to a composition, including a composition described above, wherein the nucleic acid is pgWizGFP plasmid.

In another embodiment, the present invention is directed to a composition, including a composition described above, wherein the macromolecule is a vaccine and the vesicle further includes an adjuvant.

In a further embodiment, the present invention is directed to a composition, including a composition described above, in oral dosage form wherein the vesicles are enclosed in a capsule with enteric coating to release the vesicles at a pH of 7.0 to 7.8.

In another embodiment, the present invention is directed to a composition, including a composition described above, wherein the vesicles are released from the capsule at a pH of 7.4.

In an alternative embodiment, the present invention is directed to a composition, including a composition described above, wherein the macromolecule is an insulin and the additional molecule is a protease inhibitor.

In still another embodiment, the present invention is directed to a composition, including a composition described above, wherein the protease inhibitor is selected from the group consisting of aprotonin, soy bean trypsin (SBTI) and mixtures thereof.

In a further embodiment, the present invention is directed to a composition, including a composition described above, wherein the insulin is recombinant insulin.

In a further alternative embodiment, the present invention is directed to a composition, including a composition described above, wherein the vesicle includes a compound which is a salt of SNAC or SNAD, and said salt is selected from the group consisting of a monosodium salt, a disodium salt, and a combination thereof.

In yet another alternative embodiment, the present invention is directed to a composition, including a composition described above, further including an omega-3 fatty acid.

In an additional embodiment, the present invention is directed to a composition, including a composition described above, further including EDTA or a salt thereof.

In, yet another embodiment, the present invention is directed to a composition, including a composition described above in oral dosage form which comprises a coating that inhibits digestion of said composition in a stomach of a subject.

In yet a further embodiment, the present invention is directed to a composition, including a composition described above, wherein the coating is an enteric coating or a gelatin coating.

In still another alternative embodiment, the present invention is directed to a composition, including a composition described above, wherein the fatty acid is selected from the group consisting of Myristoleic acid, Palmitoleic acid, Sapienic acid, Oleic acid, Elaidic acid, Vaccenic acid, Linoleic acid, Linoelaidic acid, α-Linolenic acid, Arachidonic acid, Eicosapentaenoic acid, Erucic acid, Docosahexaenoic acid, Caprylic acid, Capric acid, Laurie acid, Myristic acid, Palmitic acid, Stearic acid, Arachidic acid, Behenic acid, Lignoceric acid, Cerotic acid or a mixture thereof.

Another embodiment is directed to, a method of manufacturing a plurality of macromolecule loaded lipid vesicles wherein the outer surface coating of the vesicles comprises at least one cholesteryl ester obtained from cholesterol and a C₈-C₂₆ fatty acid (often C₆-C₂₂, C₈-C₁₈, more often C₈ to C₁₄ cholesteryl esters), comprising the steps of:

1. mixing by sonication
a) a polar solvent mixture containing one or more macromolecules and optionally, at least one surface modifier of said macromolecule(s) and
b) a non-polar solvent mixture consisting essentially of at least one non-ionic cholesteryl fatty acid ester selected from the group consisting of cholesteryl myristate and cholesteryl laurate

wherein said polar solvent mixture a) and said non-polar solvent mixture b) are sonicated until said one or more macromolecule, said surface modifier(s), said at least one cholesteryl fatty acid ester, said non-polar solvent and said polar solvent form a homogenous dispersion of vesicles after said mixing; and,

2. evaporating said non-polar solvent leaving said vesicles in said polar solvent,

wherein each of said vesicles comprises an exterior layer consisting essentially of a plurality of non-ionic cholesteryl fatty acid ester molecules and a hollow compartment containing said macromolecule(s).

In another embodiment, the invention is directed a method, including, the method described above, wherein the polar solvent comprises insulin in buffer at a pH ranging from 2.5 to 3.5 (preferably 3), the initial concentration of insulin in the buffer ranges from 7.5 to 8.5 mg/ml, preferably 8mg/ml, the temperature of the buffer is between 35-39 (preferably 37 degrees centigrade) and the mixture of the buffer (a) and the non polar solvent composition b) is sonicated for 20 minutes.

In another embodiment, the invention is directed a method, including the method described above, comprising subjecting the plurality of core loaded vesicles during and/or after step 1 (mixing by sonication) or step 2 (evaporating) to a dispersion step to prevent aggregation.

In another embodiment, the invention is directed a method, including the method described above, wherein the dispersion step is carried out using sodium lauryl sulfate.

In still an additional embodiment, the invention is directed a method, including the method described above, comprising subjecting the plurality of core loaded vesicles during and/or after step 1 (mixing by sonicating), step 2 (evaporating) or the dispersion step to a stabilization step employing a gelatin suspension.

In additional embodiments, the present invention is directed to composition for treating cancer.

In an embodiment, the invention is directed a composition comprising a tumor lysate or a tumor antigen encapsulated in a lipid vesicle wherein the outer surface coating of the vesicle is comprised of one or more cholesteryl esters obtained from cholesterol and a C₆-C₂₆ fatty acid (preferably, for example, a C₆-C₂₂ fatty acid, a C₈-C₂₂ fatty acid, or a C₈-C₁₄ fatty acid), wherein the outer surface coating of the vesicle remains intact during passage of said composition across a cell membrane such that the vesicle and its core enters into lymphoid cells, including dendritic cells without endosomal formation, and releases said encapsulated lysate or antigen in the cells by the action of cellular cholesteryl ester hydrolases, wherein the intracellular concentration of the lysate or antigen in the cell is at least 10 fold (often at least 100 fold) and more often at least 250 fold greater than the intracellular concentration that would be obtained from the same concentration of tumor lysate or antigen being delivered to the cells if the lysate or antigen were not encapsulated in the vesicle.

In an additional embodiment, the present invention is directed to a composition, including a composition described above, wherein the tumor lysate comprises macromolecules which are selected from the group consisting of proteins, peptides, nucleic acids or mixtures thereof and the antigen is a peptide,

In another embodiment, the present invention is directed to a composition, including a composition described above, in oral dosage form comprising a coating targeted to release the tumor lysate or antigen at a pH of 7.3 to pH 7.6, wherein said tumor lysate or the antigen is directed to dendritic cells in the ileum of a patient or subject.

In a further embodiment, the present invention is directed to a composition, including a composition described above, wherein the tumor lysate or the antigen is autologous.

In yet another embodiment, the present invention is directed to a composition, including a composition described above, wherein the ileum released composition additionally contains a cholestosome encapsulated adjuvant comprised of one or more substances demonstrated to activate dendritic cells.

In an additional embodiment, the present invention is directed to a composition, including a composition described above, wherein the adjuvant is lipopolysaccharide (LPS).

In yet a further embodiment, the present invention is directed to a composition, including a composition described above, wherein one or more checkpoint inhibitor monoclonal antibodies in an effective amount is optionally encapsulated in the vesicles, wherein the checkpoint inhibitor is nivolumab, pembrolizumab, atezolizumab or a mixture thereof and the composition optionally includes an adjuvant comprised of one or more substances demonstrated to activate dendritic cells.

In an additional embodiment, the present invention is directed to a composition, including a composition described above, wherein the adjuvant is lipopolysaccharide (LPS).

In an another embodiment, the present invention is directed to a composition, including a composition described above, wherein the ileum released composition additionally contains a cholestosome encapsulated stimulating substance for dendritic cells, preferably IMO-2125, and wherein the activation of the dendritic cells by the substance is demonstrated by an increase in the concentration of interferon gamma after exposure of dendritic ails to the composition.

In an additional embodiment, the present, invention is directed to a composition, including a composition described above, wherein the source of the tumor lysate is allogeneic or from a patient other than patient to whom the composition is to be administered,

In an alternative embodiment, the present invention is directed to a composition, including a composition described above, wherein the vesicle comprises a tumor antigen, rather than a tumor lysate.

In an additional embodiment, the present invention is directed to a composition, including a composition described above, wherein the tumor antigen is gp100 melanoma tumor antigen.

In still a further embodiment, the present invention is directed to a composition, including a composition described above, wherein the antigen is a poly-neo-epitope mRNA cancer immunotherapy antigen construct which is autologous, wherein the antigen is derived from cancer cells of the patient to be treated.

In yet another embodiment, the present invention is directed to a composition, including a composition described above, wherein said poly-neo-epitope mRNA cancer immunotherapy antigen construct is allogeneic, wherein the antigen is derived front cancer cells of a type specified but not obtained from the patient to be treated.

In another embodiment, the present invention is directed to a method of treating cancer in a patient in need comprising orally administering a composition as described above to the patient, wherein the composition activates a cellular immune response against cancer cells in said patient.

A further embodiment of the present invention is directed to a method, including a method as described above of treating cancer in a patient in need comprising direct injection of an effective amount of a composition as described above into the patient's tumor, wherein the composition activates a cellular immune response against cancer cells in the patient.

In an alternative embodiment, the present invention is directed to a method, including a method as described above of treating cancer in need wherein the cellular immune response is detected in vitro by release of IL-2 or IFN gamma in response to stimulation by the composition when the composition is exposed to the patient's T cells.

In still a further embodiment, the present invention is directed to a composition, including a composition as described above, wherein the vesicle is comprised of cholesteryl myristate and one or more of cholesteryl laurate or cholesteryl palmitate.

### BRIEF DESCRIPTION OF THE FIGURES

FIG. 1. Diagram showing assembly of a lipid, vesicle from cholesteryl myristate, cholesteryl mate and insulin in the hollow core. There are two lattice models in the format of a railroad track. The distance across the tails portion is about 20 A. (2 nm), in a liposome, it would be longer since the tails do not interdigitate but lie end to end. The distance longest of the insulin monomer is 37 A (3.7 nm) (magenta ball) The distance across the screen is 386.836 A or 38.6 nm. In the single lattice (RR track model), there is no real conformational flexibility for any molecules to move around in.

FIG. 1A, Table 1 shows a comparison of properties between Cholestosomes and alternative delivery modalities. Properties establish that cholesosomes are superior or at least equal in all categories. One particularly important aspect of this comparison is that nearly any molecule can be encapsulated into a cholestosome without altering the molecule itself in most cases, the molecule must be modified to meet the needs of the delivery method. Design flexibility is an advantageous property for a drug delivery system. Cholestosomes are not subject to most of the limitations of delivery modalities in the prior art.

FIG. 1B, Table 3. Summary of additional Preparations of cholesteryl esters, obtained after mixing various molar ratios in two different solvents at different temperatures in order to arrive at cholestosomes of known vesicle diameter

FIG. 2. Cholestosomes made by combining two short chain Cholesteryl esters that differ by two CH₂, units, Cholesteryl Caprylate (C₆) and Cholesteryl Caprate (C₈), mixed in a 1:1 molar ratio. The cholestosomes resulting from this combination were an average size of 350 nm. When FITC was incorporated into these cholestosomes and tested on MCF-7 cells, no loss of viability occurred, and the green fluorescence showed that these cholestosomes concentrated in the cells.

FIG. 3. Cholestosomes made by combining two long chain Cholesteryl esters that differ by four CH₂ units. Cholesteryl Stearate (C₁₈) and cholesteryl Behenate (C₂₂), mixed in a 1:1 molar ratio. The cholestosomes resulting from this combination ranged in size from 392 nm if prepared at 65° C. In chloroform to 3899 nm if prepared at 55° C. In ether. When FITC was incorporated into these cholestosomes and tested on MCF-7 cells, no loss of viability occurred and the green fluorescence showed that these cholestosomes concentrated in the cells.

FIG. 4. Cholestosomes made by combining two Cholesteryl esters that differ by ten CH₂ units, Cholesteryl Laurate (C₁₂) and Cholesteryl Behenate (C₂₂), mixed in a 1:4 molar ratio. The cholestosomes resulting from this combination had an average size of 1500 nm when prepared in chloroform at 65° C. When FITC was incorporated into these cholestosomes and tested on MCF-7 cells, no loss of viability occurred and the green fluorescence showed that these cholestosomes concentrated in the cells.

FIG. 5. Cholestosomes made by combining two Cholesteryl esters that differ by Eight CH₂ units, Cholesteryl Myristate (C₁₄) and Cholesteryl Behenate (C₂₂), mixed in a 1:4 molar ratio. The cholestosomes resulting from this combination had an average size of 690 nm when prepared at 65° C. When FITC was incorporated into these cholestosomes and tested on MCF-7 cells, no loss of viability occurred and the green fluorescence showed that these cholestosomes concentrated in the cells.

FIG. 6. Cholestosomes made by combining two Cholesteryl esters that differ by two CH2 units, Cholesteryl Myristate (C14) and Cholesteryl Palmitate (C₁₆), mixed in a 1:1 molar ratio. The cholestosomes resulting from this combination had an average size of1890 nm when prepared in chloroform at 65° C. When FITC was incorporated into these cholestosomes and tested on MCF-7 cells, no loss of viability occurred and the green fluorescence showed that these cholestosomes concentrated in the cells.

FIG. 7 Testing of insulin Cholestosome formulation 1117—Week 6 vs Week 18. Stability of the cholestosomes containing insulin in the refrigerator; Serial sampling of total, pellet and supernatant by ELISA assays

FIG. 8. Insulin Formulation 1117. NICOMP distribution between two distinct populations of vesicles—mean size vesicle #1 (33% of vesicles)=mean 208 nm; SD=29.2; Vesicle mean size #2 (67% of vesicles) mean 1191 nm, SD 133

FIG. 9. Average lipid concentration (n=2) in Insulin-Cholestosomes made at indicated pH and ionic strength

FIG. 10. shows that Average Insulin concentrations in Insulin cholestosome vesicles were highest when the pH of the preparation was 3.0, at lower ionic strength of buffer,

FIG. 11. Encapsulation Efficiency (E.E.) of formulations at pH (6-8) is maximized at higher ionic strengths. E.E. of pH (3&5) Cholestosomes is maximized using the ionic strength of 1× PBS.

FIG. 12. pH 3 Ins-Cholestosome vesicle sizes as determined by DLS as a function of ionic strength in the presence of Insulin.

FIG. 13. pH 3-8 Ins-Cholestosomes vesicle size as determined by DLS as a function of ionic strength in the presence of Insulin. Note the difference in size at acidic and basic conditions

FIG. 14. Reaction mixture: insulin-cholestosomes before surfactant

FIG. 15. Reaction mixture: insulin-cholestosomes after adding surfactant

FIG. 16. Reaction mixture insulin-cholestosomes after surfactant and after filtration to remove unencapsulated Insulin—Size Distribution

FIG. 17. Transmission EM of insulin cholestosome formulation 1117, at 20×, 80× and 100×, showing the larger center, interpreted as a hydrophilic pocket

FIG. 18 Illustration of the apparatus (Biocoat (Becton:Dickinson) transwell assay with CaCo-2 cells) used to collect basolateral fluids following exposure of the apical side of a monolayer Caco2 cells to a cholestosome encapsulated molecule Cells were given either FITC cholestosome insulin or unencapsulated FITC insulin, which was added to the media on the apical side of the differentiated Caco-2 monolayer. The cells were induced to form chylomicrons as described in methods. Cholestosome encapsulated molecules of all sizes are taken into Caco-2 cells, and from there are incorporated into chylomicrons by the Golgi apparatus. The uptake process by enterocytes is more rapid and efficient than the process shown here for Caco-2 cells.. Other typical components of Chylomicrons are APO-B, other apolipoproteins, and triglycerides. After formation, chylomicrons are secreted by Caco-2 cells into the lymphatic fluid on the basolateral side of the monolayer. Chylomicrons loaded with cholestosomes are captured in the fluid on the basolateral side of the Caco2 monolayer.

FIG. 19. Caco-2 Test: 2000 nm particle with FITC insulin alone and Cholestosome Encapsulated; Time=0 is baseline.

FIG. 20. Caco-2 Test: 2000 nm particle with FITC insulin alone and Cholestosome Encapsulated; Time=24 h

FIG. 21. Caco-2 Test: 2000 nm particle with FITC insulin alone and Cholestosome Encapsulated; FITC readings on Apical and basolateral sides of the caco2 monolayer, in this case at Time=24 h

FIG. 22. Cholestosome-mediated delivery of FITC-Cholestosome insulin into MCF-7 cells. Image A shows the phase contrast microscopy of the MCF-7 cells loaded with FITC-Cholestosomes after 24 hours. Image B shows the fluorescence of the MCF-7 cells loaded with FITC-Cholestosome insulin after 24 hours.

FIG. 23. compares the ability of free FITC-insulin, row A. FITC-Cholestosome Insulin row B, and FITC-Cholestosome Insulin-chylomicrons, row C, to deliver FITC-insulin into MCF-7 cells. The first column is darkfield, the second fluorescence and the third column is an overlay. The loading was nearly 1000× greater from FITC insulin cholestosome chylomicrons as shown in row C. In this experiment FITC insulin cholestosome loading of MCF-7 cells was improved over some of our previous experiments with FITC insulin cholestosomes, and here the loading was even greater from FITC insulin cholestosome chylomicrons. In all cases, processing of FITC insulin cholestosomes by Caco-2 cells into chylomicrons, produces a robust improvement in the amount of insulin inside cells. Not only are the cell membranes dramatically more concentrating FITC insulin in this image, but the cytoplasm of these cells is loaded with FITC insulin, and there is even distribution without an endosome visible at 2 hrs.

FIG. 24. insulin in Cholestosomes: Average Bioavailability of total Insulin Mice was 66%. Insulin-Cholestosome prep 1117. Mouse dose was 1 U/kg with 2 mice per data point. The Late peaks are caused by Cellular exocytosis Involving both free and total insulin

FIG. 25. Insulin in Cholestosomes: Bioavailability of free Insulin which after correction for the free insulin in the IV preparation. The IV cholestosome-Insulin was comprised of 3% free insulin; (Thus IV dose was 3% higher and correspondingly 3% lower for oral because free insulin in the GI tract is not absorbed. Insulin-Cholestosome prep 1117; Mouse dose was 1 U/kg, there were 2 mice per data point.

FIG. 26. FITC insulin concentrations in plasma and target tissues of:mice at 6 hr after dosing. Cholestosome encapsulated FITC insulin shows high absorption orally and higher tissue distribution from oral dosing than the same dose given IV. The assays reflect high concentration of FITC in cells at a time when plasma FITC concentration is low.

FIG. 27. Insulin concentration vs time in rats given 1 unit/kg cholestosome insulin 1412 formulation orally. AUC in this study was 295.7.

FIG. 28. Insulin concentrations vs time for Rats given Subcutaneous Human recombinant insulin 1.0 u/kg. AUC was 314.4

FIG. 29. Insulin concentration vs time in Rats given Insulin cholestosomes at a dose of 1.0 u/kg subcutaneously. AUC for insulin was 322.5

FIG. 30. FITC insulin concentrations in plasma and target tissues of rats at 4 hr after dosing. Cholestosome encapsulated FITC insulin shows high absorption orally and higher tissue distribution from oral dosing than the same dose given SC. The assays reflect high concentration of FITC in cells at a time when plasma FITC concentration is low.

FIG. 31. Aggregation and degradation control data on Trastuzumab (Herceptin) where absorbance at 280 nm is compared to degradation detecting wavelength (350 nm) using JENWAY spectrophotometer (Dilution factor 7.5) Note the minimal aggregation/degradation due to encapsulation, freeze-drying and dialysis.

FIG. 32. Trastuzumab-cholestosome and IgG-cholestosome preparations were analyzed for lipid content, antibody content, and size before a 1.5 ml portion was mixed with protein G-Sepharose to separate free antibodies from cholestosomes. Lipid and antibody concentrations were determined as described in Methods. *Determined by difference; **As lipid, after elution from protein G-Sepharose

FIG. 33. Growth (A) of trastuzumab-Cholestosome (1510)-treated, and IgG-Cholestosome (1519)-treated 184B5 (blue bars) and MCF-7 (grey bars) cells.

FIG. 34. Cell viability (B) of trastuzumab-Cholestosome (1510)-treated, and IgG-Cholestosome (1519)-treated 184B5 (blue bars) and MCF-7 (grey bars) cells. Cells were grown as described and treated as described in methods.

FIG. 35. Analysis of trastuzumab after concentration by lyophilization middle bar and subsequent dialysis (third bar). Trastuzumab stock solution concentration was measured after lyophilization and dialysis as described. Values above the bars represent amount of protein detected (as a percentage of the input, i.e. Untreated, set as 100%). Protein amounts were determined by comparison to a trastuzumab standard curve.

FIG. 35A, Table 4. Qiagen Buffer components per column, as used in the preparation of pgWizGFP

FIG. 36 Cholestsomes made from cholesteryl my state and cholesteryl palmitate in a 1:1 molar ratio loading human retinal epithelial cells with FITC. Panel A shows images of ARPE-19 cells treated for 2 h with FITC-Cholestosomes made from myristate/palmitate. Top left panel is the phase contrast image; the top right is the green fluorescent channel image at 2 hr, the bottom left panel is the red fluorescent channel image; the bottom right panel is the merged image.

FIG. 37. Shown are images of MCF7 cells treated with pgWizGFP Cholestosomes made from Cholesteryl myristate/palmitate (top panel at 30 hr) and pgWizGFP Cholestosomes made from Cholesteryl myristate/laurate (bottom panel at 24 hr). In each panel, the far-left image is phase contrast, the next is green fluorescent channel, the next is red fluorescent channel, and the far right is the merged image. The images show that both formulations load MCF7 cells with pgWizGFP plasmid, with expression of Green Fluorescence. Media control panels (not shown) had negligible fluorescence, consistent with background autofluorescence.

FIG. 38. Shown are images of ARPE-19 Human retinal cells treated for 24 h with pgWizGFP Cholestosomes made from Cholesteryl myristate/palmitate (top panel) and pgWizGFP Cholestosomes made from Cholesteryl myristate/laurate (middle panel) and media alone (bottom panel). In each panel, the far-left image is phase contrast, the next is green fluorescent channel, the next is red fluorescent channel, and the far right is the merged image. The images show that both formulations load ARPE-19 cells with pgWizGFP plasmid, resulting in the expression of Green Fluorescence at 24 hr. Media alone as well as pgWizGFP alone (not shown) show marginal fluorescence at 24 hr, indicating little background autofluorescence and negligible entry of unencapsulated plasmid into cells.

### DETAILED DESCRIPTION OF THE INVENTION

The following terms are used throughout the specification to describe the present invention. Where a term is not given a specific definition herein, that term is to be given the same meaning as understood by those of ordinary skill in the art. The definitions given to the disease states or conditions which may be treated using one or more of the lipid vesicle encapsulated compounds according to the present invention are those which are generally known in the art.

It is noted that, as used in this specification and the appended claims, the singular forms “a,” “an,” and “the,” include plural referents unless expressly and unequivocally limited to one referent. Thus, for example, reference to “a compound” includes two or more different compounds. As used herein, the term “include” and its grammatical variants are intended to be non-limiting, such that recitation of items in a list is not to the exclusion of other like items that can be substituted or other items that can be added to the listed items.

The term “patient” or “subject” is used throughout the specification to describe an animal, preferably a human, to whom compositions according to the present invention are administered. For administration of compositions to a specific animal such as a domestic animal (for veterinary applications) or a human patient, the term patient or subject refers to that specific animal.

The term “compound” is used herein to refer to any specific chemical compound disclosed herein. Within its use in context, the term generally refers to a single compound and its pharmaceutically acceptable salts as disclosed herein, but in certain instances may also refer to stereoisomers and/or optical isomers (including racemic mixtures) of disclosed compounds.

The term “consisting essentially of” is used to describe components of a composition or components of an element of a composition which contain those components and any additional components which do not materially change the basis and novel characteristics of the composition or element which principally, and in some cases, exclusively, contains those components.

As used in this application, the terms “about” and “approximately” are used as equivalents. Any numerals used in this application with or without about approximately are meant to cover any normal fluctuations appreciated by the skilled practitioner in the relevant aspect of the present invention.

Approximately or about: As used herein, the term “approximately” or “about,” as applied to one or more values of interest, refers to a value that is similar to a stated reference value. In certain embodiments, depending on context, the term “approximately” or “about” refers to a range of values that fall within 15%, 14%, 13%, 12%, 11%, 10%, 9%, 8%, 7%, 6%, 5%, 4%, 3%, 2%, 1%, or less in either direction (greater than or less than), more often 5% or less of the stated reference value unless otherwise stated or otherwise evident from the context (except where such number would exceed 100% or be less than 0% of a possible value).

The term “treating” refers to curing or at, least favorably impacting a disease. In another embodiment within context, “treating” refers to preventinglreducing the likelihood of a disease and/or condition, in another embodiment, “treating” refers to reducing the incidence of a disease. In another embodiment, “treating” refers to ameliorating symptoms of a disease. In another embodiment, “treating” refers to inducing remission of a disease or condition. In another embodiment, “treating” refers to slowing the progression of a disease.

### Means of Choice of Fatty Acids in the Practice of the Invention

Fatty acids suitable for use in the practice of the invention are listed in table 2 below, and are further characterized by structure, ratio of carbons to number of double bonds, the ratio of C:D and position of the double bonds:

TABLE 2
Listing of fatty acids potentally used to form cholesteryl esters
characterized by structure, ratio of Carbons to number of double bonds the ratio C:D
and position of the double bonds

| | | | Position |
|---|---|---|---|
| | | | of double |
| Common name | Chemical structure | C:D | bond |
| Myristoleic acid | CH₃(CH₂)₃CH═CH(CH₂)₇COOH | 14:1 | n-5 |
| Palmitoleic acid | CH₃(CH₂)₅CH═CH(CH₂)₇COOH | 16:1 | n-7 |
| Sapienic acid | CH₃(CH₂)₈CH═CH(CH₂)₄COOH | 16:1 | n-10 |
| Oleic acid | CH₃(CH₂)₇CH═CH(CH₂)₇COOH | 18:1 | n-9 |
| Elaidic acid | CH₃(CH₂)₇CH═CH(CH₂)₇COOH | 18:1 | n-9 |
| Vaccenic acid | CH₃(CH₂)₅CH═CH(CH₂)₉COOH | 18:1 | n-7 |
| Linoleic acid | CH₃(CH₂)₄CH═CHCH₂CH═CH(CH₂)₇COOH | 18:2 | n-6 |
| Linoelaidic acid | CH₃(CH₂)₄CH═CHCH₂CH═CH(CH₂)₇COOH | 18:2 | n-6 |
| α-Linolenic acid | CH₃CH₂CH═CHCH₂CH═CHCH₂CH═CH(CH₂)₇COOH | 18:3 | n-3 |
| Arachidonic acid | CH₃(CH₂)₄CH═CHCH₂CH═CHCH₂CH═CHCH₂CH═CH(CH₂)₃COOH^{NIST} | 20:4 | n-6 |
| Eicosapentaenoic | CH₃CH₂CH═CHCH₂CH═CHCH₂CH═CHCH₂CH═CHCH₂CH═CH(CH₂)₃COOH | 20:5 | n-3 |
| acid | | | |
| Erucic acid | CH₃(CH₂)₇CH═CH(CH₂)₁₁COOH | 22:1 | n-9 |
| Docosahexaenoic | CH₃CH₂CH═CHCH₂CH═CHCH₂CH═CHCH₂CH═CHCH₂CH═CHCH₂CH═CH(CH₂)₂COOH | 22:6 | n-3 |
| acid | | | |
| Caprylic acid | CH₃(CH₂)₆COOH | 8:0 | |
| Capric acid | CH₃(CH₂)₈COOH | 10:0 | |
| Lauric acid | CH₃(CH₂)₁₀COOH | 12:0 | |
| Myristic acid | CH₃(CH₂)₁₂COOH | 14:0 | |
| Palmitic acid | CH₃(CH₂)₁₄COOH | 16:0 | |
| Stearic acid | CH₃(CH₂)₁₆COOH | 18:0 | |
| Arachidic acid | CH₃(CH₂)₁₈COOH | 20:0 | |
| Behenic acid | CH₃(CH₂)₂₀COOH | 22:0 | |
| Lignoceric acid | CH₃(CH₂)₂₂COOH | 24:0 | |
| Cerotic acid | CH₃(CH₂)₂₄COOH | 26:0 | |

In the above table 2, C is the number of carbons and D is the number of double bonds in the alkyl chain of the fatty acid molecule, C:D ratio of the molecule as displayed. The position of the double bond is expressed as the number of carbon after the carbonyl, which is position 1 in the chain. In this manner, n−5 for myristoleic acid means that the double bond is found at position 14-5=position 9

### Cholesterol for Esterification in the Practice of the Invention

The tent “cholesterol” is used in the present invention to describe any cholesterol compound which may be condensed with a fatty acid in the preparation of the cholesteryl esters which may be used to form cholestosomes pursuant to the present invention. The term “cholesterol” and includes the molecule identified as cholesterol itself, and any related cholesterol molecule with additional oxygenation sites (“an oxygenated analog of cholesterol”) as m for example but not limited to), 7-ketocholesterol, 25-hydroxy cholesterol, 7-beta-hydroxycholesterol, cholesterol, 5-alpha, 6-alpha epoxide, 4-beta hydroxycholesterol, 24-hydroxycholesterol, 27-hydroxycholesterol, 24,25-epoxycholesterol, Oxysterols can vary in the type (hydroperoxy, hydroxy, keto, epoxy), number and position of the oxygenated functions introduced and in the nature of their stereochemistry. These various cholesterols may be used to provide cholesterol esters Which vary in solubility characteristics so as to provide some flexibility in providing a cholestosome with a neutral surface and groups which can instill hydrophilicity in the cholesterol ester molecules. The cholesterol type molecule could also include any sterol structurally based compound containing the OH necessary for ester formation such as Vitamin a

Molar ratios claimed in beneficial formation of cholestosomes range from 0.05 to 0.95 of any pair of esters (when a pair of esters is used) listed in table 2 above. Product ratios of composition between pairs of approximately equal alkyl chain length cholesteryl esters and active molecules range from about 2:2:96 to 48:48:4, often 45:45:10 to about 2:2:96, about 40:40:20 to about 5:5:90, about 40:40:20 to about 25:25:50. It is noted that in many cholestosome formulations when two (or more) cholesteryl esters are used, the ratio may vary above or below a 1:1 ratio (e.g. 60:40 to 40:60 or 55:45 to 45:55) for the cholesteryl esters used.

### Means of Choice of Cholesteryl Esters in the Practice of the Invention

By means of example, the following principles define the basis for choice of a component cholesteryl ester in a cholestosome, a means of choosing an ester or ester pair for encapsulation purposes, and rely on the disclosed physiochemical, properties of the listed cholesteryl esters in Table 2:
1) The esters chosen for combination should be able to arrange themselves to optimize the ester link interactions between ester pairs. This electrostatic interaction is important for orientation purposes, with the necessary hydrophobic exterior and hydrophilic center of, the vesicle,
2) The alkyl interactions should be able to optimize van der Waals forces,
3) The sum of electrostatic interactions and the alkyl interaction van der Waals forces are fundamental properties that hold the vesicle shape and thereby retain the molecule inside. A key additional factor for stability of cholestosome vesicles includes the degree of repulsion between the dual hydrophobic ends of the esters and the aqueous component containing the molecule(s) to be encapsulated.
4) The overall size of the vesicle becomes a function of the length of the alkyl chain. The increased length of the esters chosen will increase the overall hydrophobic character of the entire vesicle.
5) Using smaller chain length esters will actually increase the overall hydrophilic character of the vesicle (in terms of the overall structure of each ester).
6) Molecules that require more hydrophobic areas to assist in encapsulation within the vesicle could benefit from esters having longer alkyl chains.
7) Molecules that are smaller and require more hydrophilic components to assist in encapsulation would benefit from ester pairs that are shorter in length.
8) An additional choice is the use of unsaturated alkyl chains such as those listed in Table 2, where these fatty acids are used to prepare ester side chains for use in forming cholesteryl esters.
9) The use of an unsaturated fatty acid offers an additional structural modification in the vesicle structure which incorporates additional electrostatic interactions between the aqueous and the double bond character.
10) In the process of selection of esters for vesicle formation, selection of CH₂ chain lengths ranging for example from 2 CH₂ units but less than 27 CH₂ in length result in a structure that may not be as tight, as a result of the challenges in adapting the alkyl chains to maximize their interactions in a vesicle. The cholesterol component of the vesicle wall does not change. The van der Waals interactions within CH₂ units governs the flexibility of the alkyl interactions. However, for beneficial hydrophilic vesicle center, the optimal configuration in this vesicle is longer alkyl chains, meaning that larger ester molecules have greater utility for stabilizing more hydrophilic vesicle centers of the vesicle exposed to the aqueous environment in formulation stability.
11) The choice of paired cholesteryl esters is made on relative affinity of the fatty acid to the paired fatty acid transporters on the duodenal enterocytes. On Caco2 cells, these transporters are more avidly taking up shorter chain fatty acids compared to longer chains

Cholesteryl esters are selected for the composition of the vesicle, based on their reactivity with cholesterol transporters on the surface of duodenal intestinal (duodenal) enterocytes, which facilitates their rapid and complete uptake into the enterocytes. An essential step in the practice of the invention is uptake by the apical surface transporters that are uniquely expressed on enterocytes ( 14). The further essential step is uptake of the intact two ester cholesteryl ester vesicle by these transporters, which are also found on Caco-2 cells. (15). The inventors have unexpectedly discovered that vesicles made of different cholesteryl esters are taken up by these transporters. Because this uptake does not involve an endosome and because these transporters do not break open the cholesteryl ester vesicle, the materials chosen have unexpectedly resulted in an intact vesicle which was produced from more than one cholesteryl ester. Thus there is no alteration of the vesicle or its contents during entry into enterocytes. Further to the practice of the invention, specific combinations, of cholesteryl ester vesicles can be assembled to take advantage of the optimal functioning of these transporters, where shorter chain cholesteryl esters such as caprate, caprylate, myristate and laurate are taken up more avidly and completely than longer chain esters such as palmitate, oleate, stearate and behenate.

Once inside, cholesteryl ester vesicles offer the added benefit of a protection of the payload contents of the vesicle during chylomicron formation inside the enterocyte, since the enterocytes are among the few body cells that do not hydrolyze cholesteryl esters back into the cholesterol and fatty acid components. This feature and the chylomicron formation step is a unique property of enterocytes, and thus an essential step in the practice of the invention disclosed herein, since pairs of cholesteryl esters are chosen by the inventors to optimize both loading of molecule and to relative affinity for the apical transporter of the enterocyte.

Regular cells (herein considered cells that are not enterocytes) also have surface transporters for the disclosed cholesteryl ester vesicles, and as the inventors have shown here, regular cells that are not enterocytes (MCF-7 cells are used as a non-limiting example) also take up cholesteryl ester vesicles of the present invention (without an endosome formation step) during passage of the cell membrane.

Surprisingly, the post cell uptake processing of cholesteryl ester vesicles differs between regular cells and enterocytes. Specifically, the arrival of the cholesteryl ester vesicle inside the cell following receptor mediated endocytosis is a signal to release of cholesteryl ester hydrolases, and the specific action of this enzyme breaks open the vesicle to release its payload into the cytoplasm. This does not happen in enterocytes, as only in these cells the intact vesicle is not hydrolyzed and is incorporated into a chylomicron in its intact form.

Additional favorable properties of the cholesteryl ester components of the vesicle are
1. their surface neutral charge allows the enterocytes to selectively recognize these components of the invention,
2. the entire composition of the lipid vesicle coating is comprised of safe dietary ingredients (fatty acid esters of cholesterol) in small amounts, thereby providing a safe delivery means, and
3. in particular on permeability and in particular, on their potential to “pack” with each other and the requirements of the pharmaceuticals to be incorporated in the vesicles themselves.

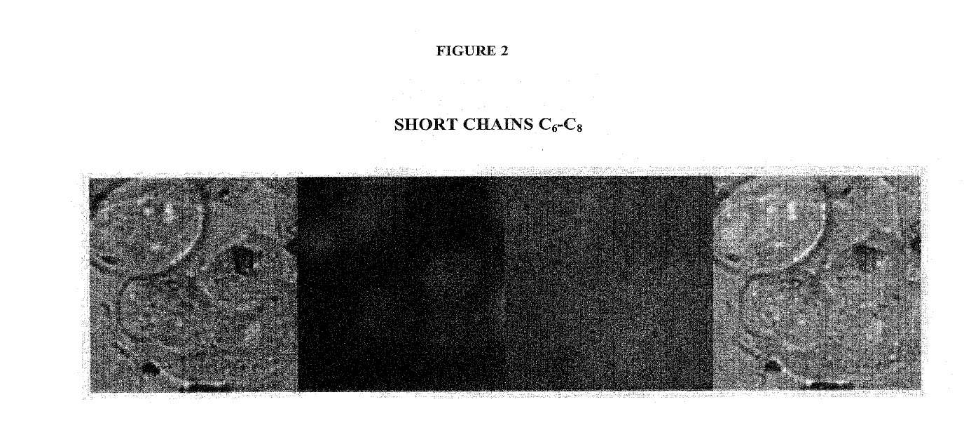

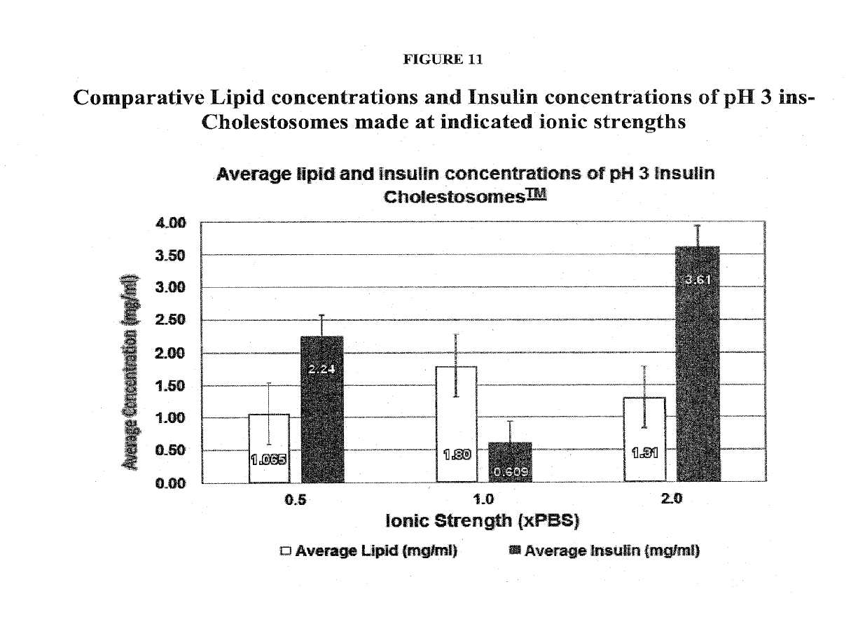

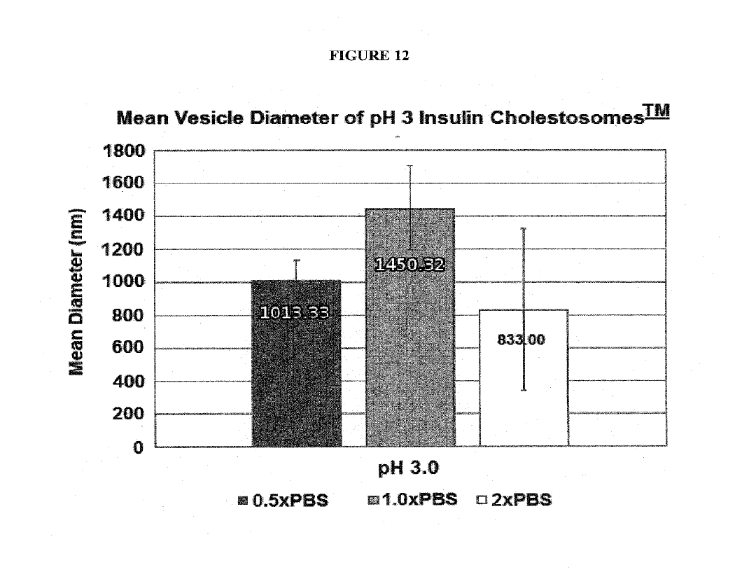

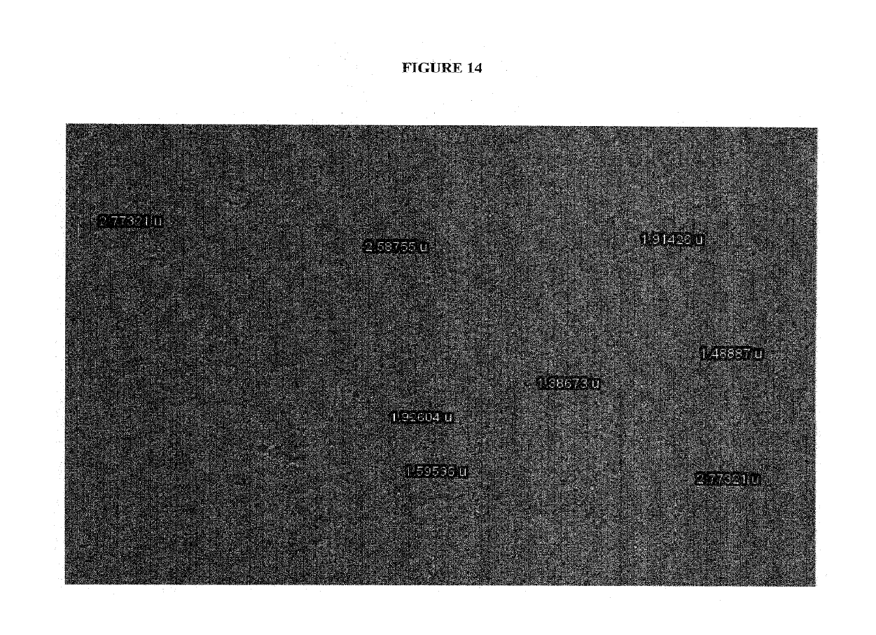

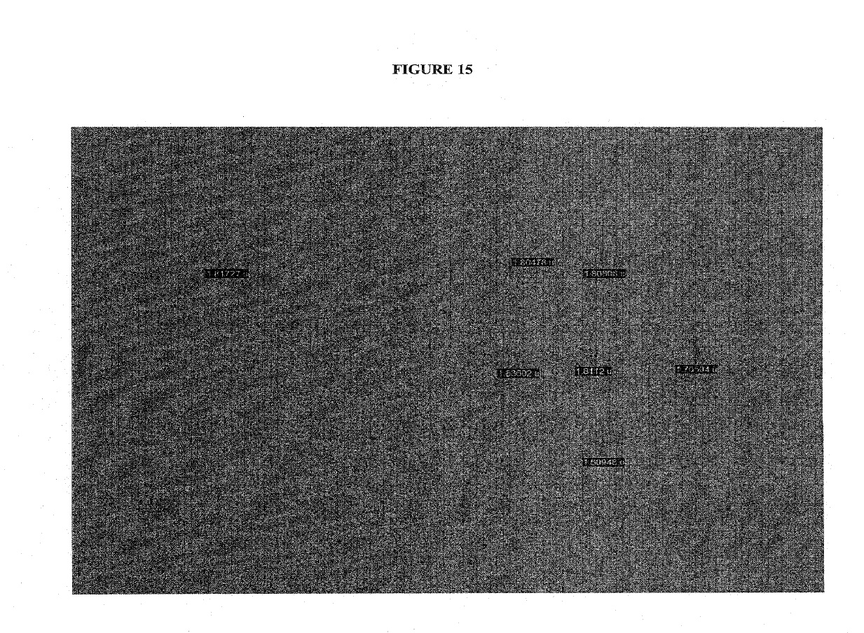

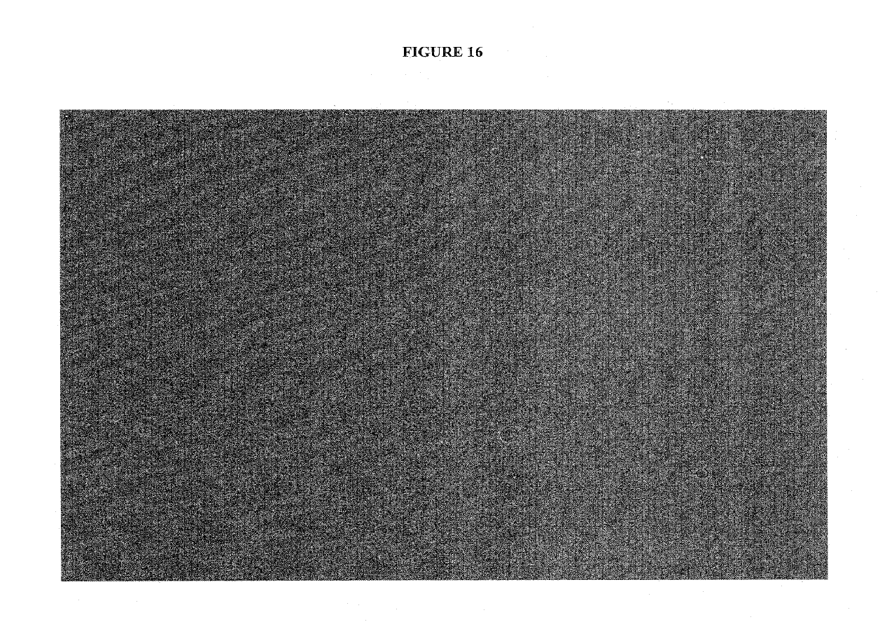

FIGS. 2-6 illustrate molecular modeling diagrams by means of an example of Cholestosome vesicle matrix formation from different pairs of cholesteryl esters selected from Table 2, In FIGS. 11-13, the representative peptide molecule was Insulin, a peptide of 6 kd size that is generally water soluble. In FIGS. 14-15, the cholestosome vesicle structure was applied to encapsulate bevacizumab, a representative monoclonal antibody of size approximately 150 kd. In FIG. 16 all 3 representative molecules are shown in relation to the cholestosome vesicle formed from cholesteryl esters myristate and laurate.

For ester pairs that are greater than 6 CH₂ units different in length (which is defined as intermediate) it is possible to maintain ester interactions and turn the molecules in opposite directions to still have alkyl chains packed into a vesicle. This arrangement would be useful for packing in molecules that have alternating structural regions of hydrophobic/hydrophilic character, and which when incorporated into said vesicle, could be relied upon to segregate different molecule types.

The choice of ester pairs is a function of the structure of the molecule needed to be encapsulated and its ability to interact with the vesicle.

### Neutral Charge of Cholestosomes vs Positive Charge of Liposomes

Liposomes are not able to pass the Caco-2 enterocyte barrier intact, in fact most are broken open in the GI tract to harvest their individual component phospholipids. Thus liposomes and their payloads are not taken up by enterocytes, perhaps due to their surface charge. Cholestosomes are comprised of Cholesteryl esters, which are in final form for absorption into duodenal enterocytes (already converted by cholesterol esterases into absorbable moieties). They are already neutral particles by virtue of their composition from cholesteryl esters, and are preferred in this term by the enterocyte cells of the duodenum for absorption intact and use in chylomicron formation. As long as the encapsulated molecule is completely within the hollow center, cholestosomes are taken up intact and they are placed intact into chylomicrons in the Golgi apparatus of enterocytes.

### Liposomes Do not Load Proteins, While Cholestosomes Load Them Preferentially

Liposomes do not load proteins, genetic materials (polynucleotides, such as DNA and/or RNA as otherwise described herein), peptides (especially including polypeptides such as monoclonal antibodies) and many macromolecules including macromolecular antibiotics in usable amounts (less than 2% means that the amount of carrier is very large if encapsulating a dose of 100-1000 mg which is typical of peptides or monoclonal antibodies). Many molecules which are water soluble, and where the charge is positive, are not favorably loaded into nanoparticles like phospholipid based liposomes. In contrast, the inside of a cholestosome (core) is large in relation to the size of the encapsulating membrane, and hydrophilic. The charge is neutral, a system compatible with loading proteins, peptides, genes as well as hydrophilic small molecules which are charged. Since all of these fail to pass the GI tract barrier, the use of Cholestosome vesicles offer, for the first time, the prospect of orally absorbed proteins and peptides that pass thru the enterocytes rather than are forced around them.

### Liposomes and Therefore Their Contents Do not Enter Chylomicrons

Phospholipid coatings of liposomes are degraded in the GI tract, and thus the liposome itself has been degraded and its contents released in the GI tract, and even before arrival at the duodenal site of absorption. Thus even if a protein could be loaded into a liposome, it would be destroyed with the liposome before it could be absorbed by emerocytes. There is no possibility for a phospholipid constituent liposome to be incorporated into a chylomicron.

### Liposomes Do not Pass Cell Membranes

Not only do liposomes fail to be orally absorbed with their payloads, they also do not enter cells and certainly when lacking APO on their surfaces, they have no ability to dock with cells in need of lipids. When injected intravenously, Liposomes are harvested by the liver and there broken down into their component phospholipids. This does not ordinarily offer intracellular delivery of their contents, although high local concentrations of payload molecules in the liver may offer an advantage lithe target cell is the hepatocyte,

### Cholesteryl Esters Useful In the Composition

A cholesteryl ester comprised of cholesterol and a fatty acid, preferably a C₄-C₁₆ fatty acid, often a C₆-C₂₆ is fatty acid or a C₆-C₂₂ fatty acid, more often a C₈-C₂₀ fatty acid, even more often a C₈-C₁₄ fatty acid. In certain embodiments, the fatty acid is selected from the group consisting Myristoleic acid, Palmitoleic acid, Sapienic acid, Oleic acid, Elaidic acid, Vaccenic acid, Linoleic acid, Linoelaidic acid, α-Linolenic acid, Arachidonic acid, Eicosapentaenoic acid, Erucic acid, Docosahexaenoic acid, Caprylic acid, Capric acid, Laurie acid, Myristic acid, Palmitic acid, Stearic acid, Arachidic acid, Behenic acid, Lignoceric acid, Cerotic acid or a mixture thereof can he suitable for use in the practice of the invention. Fatty acids of C4 to C6 are often not particularly suitable for the invention, as the inventors have discovered that these often do not form vesicles.

Lipid Particle

As defined in United States Patent Application Document No, 2011-0268653 by Negrete and colleagues (16) “‘lipidic particle’ refers to a particle having a membrane structure in which amphipathic lipid molecules are arranged with their polar groups oriented to an aqueous phase. Examples of the lipid membrane structure include configurations such as a liposome, multi-lamellar vesicle (MLV), and a micelle structure.

A ‘liposome’ refers to a closed nanosphere, which is formed by forming a bilayer membrane of a phospholipid molecule with the hydrophobic moiety positioned inside and the hydrophilic moiety positioned outside, in water and closing the ends of the bilayer membrane. Examples of liposomes include a nanosphere having a single layer formed of a phospholipid bilayer membrane and a nanosphere having a multiple layer formed of a plurality of phospholipid bilayers. Since a liposome has such a structure, an aqueous solution is present both inside and outside of the liposome and the lipid bilayer serves as the boundary.

A ‘micelle’ refers to an aggregate of amphipathic molecules. The micelle has a form in which a lipophilic moiety of this amphipathic molecules is positioned toward the center of the micelle and a hydrophilic moiety is positioned toward the outside thereof, in an aqueous medium. A center of a sphere is lipophilic and a peripheral portion is hydrophilic in such a micelle. Examples of a micelle structure include spherical, laminar, columnar, ellipsoidal, microsomal and lamellar structures, and a liquid crystal.” Note that such structures do a very poor job of encapsulating hydrophilic molecules like peptides and proteins, where loading is 1:1000 or worse. Contrast that with cholestosomes with hydrophilic centers (from the orientation of the ester functionality) and hydrophobic outsides.

In certain embodiments, the interior and exterior may be the same with the sterol nucleus on the outside surface and inside cavity with the tails of the esters interdigitated in a Pseudo-bilayer type of molecule. When a chylomicron takes up a cholestosome, the truly hydrophilic outside is re-established by the Apolipoprotein components of the transformed and loaded chylomicrons, and the Apolipoproteins also facilitate docking of the transformed chylomicrons with cells. In short, the cholestosome two stage formation into a chylomicron is totally novel and unexpected compared to previous efforts.

### Effective Amount

The term “effective amount” is used throughout the specification to describe concentrations or amounts of formulations or other components which are used in amounts, within the context of their use, to produce an intended effect according to the present invention. The formulations or component may be used to produce a favorable change in a disease or condition treated, whether that change is a remission, a favorable physiological result, a reversal or attenuation of a disease state or condition treated, the prevention or the reduction in the likelihood of a condition or disease-state occurring, depending upon the disease or condition treated. Where formulations are used in combination, each of the formulations is used in an effective amount, wherein an effective amount may include a synergistic amount. The amount of formulation used in the present invention may vary according to the nature of the formulation, the age and weight of the patient and numerous other factors which may influence the bioavailability and pharmacokinetics of the formulation, the amount of formulation which is administered to a patient generally ranges from about 0.001 mg/kg to about 50 mg/kg or more, about 0.5 mg/kg to about 25 mg/kg, about 0.1 to about 15 mg/kg, about 1 mg to about 10 mg/kg per day and otherwise described herein. For avoidance of doubt, the dosage of the component in said formulation given to said animal is approximately the same as would be given by parenteral means, after collection for the added mass of the delivery system. The person of ordinary skill may easily recognize variations in dosage schedules or amounts to be made during the course of therapy.

The term “coadministration” is used to describe the administration of two or more active compounds, in this case a compound according to die present invention, in combination with an additional agent or other biologically active agent, in effective amounts. Although the term coadministration preferably includes the administration of two or more active compounds to the patient at the same time, it is not necessary that the compounds actually be administered at the exact same time or in the same composition (although that may be preferable), only that amounts of compound will be administered to a patient or subject such that effective concentrations are found in the blood, serum or plasma, or in the pulmonary tissue at the same time.

### Active Agent

The term “active nolecule”, “active agent” or “active compound” shall mean any molecule which is active in a biological system and which maybe incorporated into a cholestosome as described herein. Cholestosomes according to the present invention are able to readily accommodate a large number of active compounds in their large neutral charged cores, including small molecules and large molecules, especially including compounds which cannot otherwise be delivered efficiently orally. This is because of the unique mechanism (as described herein) that cargo-loaded cholestosomes provide in delivering active compounds through emerocytes into chylomicrons and then into the cells of a patient or subject to whom these cargo-loaded cholestosomes are administered. These active molecules include small molecules which are unstable to standard oral delivery techniques and are typically only parenterally administered and macromolecules such as proteins (including glycoproteins) an polypeptides (e.g. insulin, interferon, hCG, C-reactive protein, cytokines, including various interleukins, growth factors), other polypeptides, including antibodies such as polyclonal antibodies, monoclonal antibodies (as otherwise described in detail herein, antibody fragments (single chain variable fragments or scFv, antigen-binding fragments or Fab, ₃G antibodies), immunogenic polypeptides and oligopeptides, polynucleotides, including DNA and RNA, such as naked DNA, plasma DNA, mRNA, siRNA, shRNA, bifunctional shRNA, microRNA (including 122, among others) and various oligonucleotides of DNA and RNA. Plasmids that can be loaded into cells and produce expression as fluorescence, including but not limited to pgWizGFP as disclosed herein, are also within the scope of active agents. Numerous anti-infective agents, including antibiotics (such as vancomycin and penicillin) and antiviral agents and other active molecules, especially including macromolecular antibiotics as well as numerous anticancer agents which are disclosed in detail herein, may also be delivered by the present invention.

It is noted that cholestosomes pursuant to the present invention may be used to deliver virtually any active molecule of a wide variety of sizes and molecular weights. Cholestosomes according to the present invention may also be used to topically deliver a number of active molecules to provide high bioavailability through the skin of a patient or subject including topical antibiotics, topical anti-fungals, topical platelet derived growth factor, other growth factors, topical anti-TNF for psoriasis, for example and topical vaccines, and topical deliver of cosmetic agents, among others. Numerous chemotherapeutic agents, antibiotics, and antiviral agents may be incorporated into cholestosomes according to the present invention. The cholestosomes according to the present invention are particularly suited for these compounds, even small molecules, because delivery of the compound into the cell pursuant to the mechanism of active molecule delivery by compositions according to the present invention represents a particularly effective therapy against a variety of microbes, including bacteria and viruses.

Other compounds for use in the present invention are also described herein below, and in the examples which follow.

The term “cancer” shall refer to a proliferation of tumor cells having the unique trait of loss of normal controls, resulting in unregulated growth, lack of differentiation, local tissue invasion, and/or metastasis. As used herein, neoplasms include, without limitation, morphological irregularities in cells in tissue of a subject or host, as well as pathologic proliferation of cells in tissue of a subject, as compared with normal proliferation in the same type of tissue. Additionally, neoplasms include benign tumors and malignant tumors (e.g., colon tumors) that are either invasive or noninvasive. Malignant neoplasms are distinguished from benign neoplasms in that the former show a greater degree of dysplasia, or loss of differentiation and orientation of cells, and have the properties of invasion and metastasis. The term cancer also within context, includes drug resistant cancers, including multiple drug resistant cancers. Examples of neoplasms or neoplasias from which tumor lysates of the present on may be derived include, without limitation, carcinomas (e.g., squamous-cell carcinomas, adenocarcinomas, hepatocellular carcinomas, and renal cell carcinomas), particularly those of the bladder, bone, bowel, breast, cervix, colon (colorectal), esophagus, head, kidney, liver, lung, nasopharyngeal, neck, thyroid, ovary, pancreas, prostate, and stomach; leukemias, such as acute myelogenous leukemia, acute lymphocytic leukemia, acute promyelocytic leukemia (APL), acute T-cell lymphoblastic leukemia, adult T-cell leukemia, basophilic leukemia, eosinophilic leukemia, granulocytic leukemia, hairy cell leukemia, leukopenic leukemia, lymphatic leukemia, lymphoblastic leukemia, lymphocytic leukemia, megakaryocytic leukemia, micromyeloblastic leukemia, monocytic leukemia, neutrophilic leukemia and stem cell leukemia, benign and malignant lymphomas, particularly Burkitt's lymphoma, Non-Hodgkin's lymphoma and B-cell lymphoma; benign and malignant melanomas; myeloproliferative diseases; sarcomas, particularly Ewing's sarcoma, hemangiosarcoma, Kaposi's sarcoma, liposarcoma, myosarcomas, peripheral neuroepithelioma, and synovial sarcoma; tumors of the central nervous system (e.g., gliomas, astrocytomas, oligodendrogliomas, ependymomas, gliobastomas, neuroblastomas, ganglioneuromas, gangliogliomas, medulloblastomas, pineal cell tumors, meningiomas, meningeal sarcomas, neurofibromas, and Schwannomas); germ-line tumors (e.g., bowel cancer, breast cancer, prostate cancer, cervical cancer, uterine cancer, lung cancer (e.g., small cell lung cancer, mixed small cell and non-small cell cancer, pleural mesothelioma, including metastatic pleural mesothelioma small cell lung cancer and non-small cell lung cancer), ovarian cancer, testicular cancer, thyroid cancer, astrocytoma, esophageal cancer, pancreatic cancer, stomach cancer, liver cancer, colon cancer, and melanoma; mixed types of neoplasias, particularly carcinosarcoma and Hodgkin's disease; and tumors of mixed origin, such as Wilms' tumor and teratocarcinomas, among others. It is noted that certain epithelial tumors including ovarian, breast, colon, head and neck, medulloblastomas and B-cell lymphoma, among others are shown to exhibit increased autophagy and are principal target cancers for compounds and therapies according to the present invention.

The term “additional anti-cancer agent” is used to describe an additional compound which may be coadministered with one or more compositions which include vesicles pursuant to the present invention in the treatment of cancer. Such agents include, for example, everolimus, trabectedin, abraxane, TLK 286, AV-299, DN-101, pazopanib, GSK690693, RTA 744,ON 0910.Na, AZD 6244 (ARRY-142886), AMN-107, TKI-258, GSK461364, AZI) 1152, enzastaurin, vandetanib, ARQ-197, MK-0457, MLN8054, PHA-739358, R-763, AT-9263, a FLT-3 inhibitor, a VEGFR. inhibitor, an EGFR TX inhibitor, an aurora kinase inhibitor, a PIK-1 modulator, a Bcl-2 inhibitor, an HDAC inhibitor, a c-MET inhibitor, a PARP inhibitor', Cdk inhibitor, an EGFR TK inhibitor, an IGFR-TK inhibitor, anti-HGF antibody, a P13 kinase inhibitors, an AKT inhibitor, a JAK/STAT inhibitor, a checkpoint-1 or 2 inhibitor, a focal adhesion kinase inhibitor, a Map kinase kinase (mek) inhibitor, a VEGF trap antibody, pemetrexed, adotinib, dasatardb, decatanib, panitumumab, amaibiein, oregovomb, Lep-etu, nolat ed, azd2171, batabulin, ofattimumah, zanolimumab, edotecarin, tetrandrine, rubitecan, tesmilifene, oblimersen, ticilimumab, ipilimumab, gossypol, Bio 111,131-1-TM-601, ALT-110, BIO 140, CC 8490, cilengitide gitnatecan, EL13-PE38QQR, INO 1001, IPdR₁ KRX-0402, lucanthone, 317615, neuradiab, vitespan, Rta 744, Sdx 102, talampanel, atrasentan, Xr 311 romidepsin, ADS- 100380, sunitinib, 5-fluorouracii, vorinostat, etoposide, gemeitabine, doxorubicin, irinotecan, liposomal doxorubicin, 5′-deoxy-5-fluorouridine, yincristine, temozolomide, 304709, seliciclib; PD0325901, AZD-6244, capecitabine, L-Glutarnic acid, N-[2-[2-(2-amino-4,7-dihydro-4-oxo-1 H-pyrrolo[2,3-d]pyrimidin-5-yl)ethyl]benzoyl]-, disodium salt, heptabydrate, camptothecin, PEG-labeled irinotecan, tamoxifen, toremifene citate, anastrazole, exemestane, letrozole, DES(diethylstilbestol), estradiol, estrogen, conjugated estrogen, bevacizumab, IMC-1C11, CHIR-258,); 3-[5-(methylsulfonylpiperadinemethyl)-indolylj-quinolone, vatalanib, AG-013736, AVE-0005, the acetate salt of [D-Ser(Bu t) 6,Azgly 10 ] (pyro-Glu-His-Trp-Ser-Tyr-D-Ser(Bu t)-Leu-Arg-Pro- Azgly-NH₂ acetate [C₅₉H₈₄N₁₈O₁₄-(C₂H₄O₂)ₓ where x=1 to 2.4], goserelin acetate, leuprolide acetate, triptorelin pamoate, medroxyprogesterone acetate, hydroxyprogesterone caproate, mecestrol acetate, raloxifene, bicalutamide, flutamide, nilutamide, megestrol acetate, CP-724714; TAK-165, HKI-272, erlotinib, lapatanib, canertinib, ABX-EGF antibody, erbitux, EKB-569, PKI-166, GW-572016, lonafamib, BMS-214662, tipifamib; amifostine, NVP-LAQ824, suberoyl analide hydroxamic acid, valproic acid, trichostatin A, FK -228, SU11248, sorafenib, KRN951, aminoglutethimide, arnsacrine anagrelide, L-asparaginase, Bacillus Calmette-Guerin (BCG) vaccine, bleomycin, buserelin, busulfan carboplatin, carmustine, chlorambucil, cisplatin, cladribine, clodronate, cyproterone, cytarabine, dacarbazine, dactinomycin, daunorubicin, diethylstilbestrol, epirubicin, fludarabine, fludrocortisone, fluoxymesterone, flutamide, gemcitabine gleevac, hydroxyurea, idarubicin, ifosfamide, imatinib, leuprolide, levamisole, lomustine, mechlorethaine, melphalan, 6-mercaptopurine, mesna, methotrexate, mitomycin, mitotane, mitoxantrone, nilutamide, octreotide, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deooxyuridine, cytosine arabinoside, 6-mecaptopurine, deoxycoformycin, calcitriol, valrubicin mithramycin vinblastine vinorelbine, topotecan, razoxin, marimastat, COL-3, neovastat, BMS-275291, squalamine, endostatin, SU5416, SU6668, EMD121974, interleukin 12, IM862, angiostatin, vitaxin, droloxifene, idoxyfene, spironolactone, finasteride, cimitidine, trastuzumab, denileukin diftitox, gefitinib, bortezimib, paclitaxel, irinotecan, topotecan, doxorubicin, docetaxel, vinorelbine, bevacizumab (monoclonal antibody) and erbitux, cremophor-free paclitaxel, epithilone B, BMS-247550, BMS-310705, droloxifene, 4-hydroxytamoxifen, pipendoxifene, ERA- 923, arzoxifene, fulvestrant, acolbifene, lasofoxifene, idoxifene, TSE-424, HMR-3339, ZK186619, PTK787/ZK 222584, VX-745, PD 184352, rapamycin, 40-O-(2-hydroxyethyl)-rapamycin, temsirolimus, AP-23573, RAD001 ABT-578, BC-210, LY294002, LY 92223, LY292696, LY293684, LY293646, wortmannin, ZM336372, L-779,450, PEG-filgrastim, darbepoetin, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritgumomab tiuxetan, androgens, decitabine, hexamethylmelamine, bexarotene, tositumoinab, arsenic trioxide, cortisone, editronate mitotane, cyclosporine, liposomal daunorubicin, Edwina-asparagmase, strontium 89, casopitant, netupitant, an NK-1 receptor antagonists, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, sspegtilgrastim, erythropoietin, epoetin alfa and darbepoetin alfa, ipilumumab, vemurafenib among others. Other anticancer agents which may be used in combination include immunotherapies such ipilimumab, pembrolizumab, nivolumab. These compounds may be administered separately with the vesicle containing compositions according to the present invention or in some cases, may be included in vesicles according to the present invention in combination with one or more macromolecules and optional additional compounds as otherwise described herein.

It is noted in the present invention that incorporation of active molecules into cholestosomes and administration to a patient or subject will produce a greater therapeutic effect at the same dosage level than identical active molecules delivered by prior art methods. In effect, the mechanism of packaging cargo-loaded cholestosomes in chylomicrons results in a substantial greater amount or concentration of an active molecule at its actual site of activity (in a cell) resulting in substantially greater efficacy than prior art methods. In many instances, the amount of concentration of active agent delivered inside a cell according to the present invention is at least 2 and often as much as 10 times to 1000 times the concentration of active compared to delivery by prior art (contemporary) means.

### Peptides Suitable for Encapsulation in Cholestosomes

In a preferred embodiment, the invention provides a peptide-loaded cholestosome pharmaceutical composition for oral or intracellular use, comprising a peptide or a protein which is often selected from the group consisting of a hydrophilic peptide, including but not limited to insulin, interferon alpha, interferon beta, human growth hormone, prolactin, oxytocin, calcitonin, bovine growth hormone, porcine growth hormone, Ghrelin, exenatide, extendin-4, GLP-1, any GLP-1 agonist, PYY36, Oxyntomodulin, GLP-2, and Glucagon, any of which is encapsulated by a cholesteryl ester as otherwise described herein. In another embodiment, the protein is an insulin secretagogue. In another embodiment, the protein is GLP-1. In another embodiment, the protein is a GLP-1 analogue. In another embodiment, the protein is a GLP-1 mimetic. In another embodiment, the protein is an incretin mimetic. In another embodiment, the protein mimics the GLP-1 incretin. In another embodiment, the protein is GLP-2, In another embodiment, the protein is a GLP-2 analogue. In another embodiment, the protein is a GLP-2 mimetic. This composition can be administered to improve structure or function of organs and tissues such as pancreas or liver, to increase or initiate growth of a mammal or to administer insulin in those individuals to whom insulin treatment is beneficial.

### Insulin, GLP-1 and Compositions for Use In the Practice of the Invention

In one embodiment, the insulin of methods and compositions of the present invention is human insulin. In another embodiment, the insulin is a recombinant insulin. In another embodiment, the insulin is recombinant human insulin. In another embodiment, the insulin is bovine insulin. In another embodiment, the insulin is porcine insulin. In another embodiment, the insulin is a metal complex of insulin (e.g. a zinc complex of insulin, protamine zinc insulin, or globin zinc insulin).

In another embodiment, the insulin is contained in the present invention in the form of a hexamer. In another embodiment, the insulin is classified as fast acting, where said classification include by example the insulin analogues insulin aspart, insulin lispro, and insulin glulisine.

In another embodiment, the insulin is classified as short-acting, where said classification includes regular insulin,

In another embodiment, the insulin is classified as long acting, were said classification includes by way of example insulin glargine, insulin detemir and insulin degludec.

In another embodiment, the insulin is lente insulin. In another embodiment, the insulin is semilente insulin. In another embodiment, the insulin is Ultralente insulin.

In another embodiment, the insulin is NPH insulin. In another embodiment, the insulin is glargine in another embodiment, the insulin is lispro insulin. In another embodiment, the insulin is aspart insulin.

In another embodiment, the insulin is a combination of two or more of any of the above types of insulin in another embodiment, the insulin is any other type of insulin known in the art. Each possibility represents a separate embodiment of the present invention.

In another embodiment, one or more of the above types of insulin may optionally be combined with an inhibitor of insulin metabolism which is able to prevent the intracellular metabolism of the insulin when it is released from the delivery means inside body cells.

In another embodiment, one or more of the above types of insulin may be combined with one or more of the above peptides or peptides not already listed above in the preferred embodiment. Any peptide known in the art is within the scope of the invention.

In metabolic syndrome, there is a known secretion of so called ileum hormones, which regulate the balance between nutritional intake, microbiome actions, and the metabolic balance. Ileum hormones therefore include, but are not limited to, GLP-1, glicentin, terminally glycine-extended GLP-1 (7-37), (PG (78-108)); intervening peptide-2 (PG (111-122) amide); GLP-2 (PG (126-158), GRPP (PG (1-30)), oxyntomodulin (PG (33-69), and other peptide fractions to be isolated, PYY (PYY 1-36) and (PYY 3-36), cholecystokinin (CCK), gastrin entero-glucagon and secretin. Any one or any combination of more than one of these peptides are suitable for encapsulation in the cholesteryl esters of the present invention.

In another embodiment, the peptide loaded cholestosome of the present invention contains one or more Insulin and one or more GLP-1 in any molar ratio as effective for the treatment of a patient in need.

In another embodiment, the peptide loaded cholestosome of the present invention contains one or more insulin and one or more glucagon molecules in any molar ratio as effective for the treatment of a patient in need.

In another embodiment, the peptide loaded cholestosome of the present invention provides a method for treating diabetes mellitus in a subject, comprising administering orally to the subject a pharmaceutical composition comprising an insulin and optionally other peptides and optionally one or more inhibitors of intracellular metabolism of said substance, thereby treating diabetes mellitus.

In another embodiment, said peptide loaded cholestosome containing one or more insulin and one or more GLP-1, optionally contains an inhibitor of intracellular insulin metabolism and optionally contains an inhibitor of intracellular GLP-1 metabolism. Any inhibitor of GLP-1 intracellular metabolism and any inhibitor of Insulin intracellular metabolism contained within the core of the cholestosome with insulin and GLP-1 would be within the scope of the invention.

In a preferred embodiment, the inhibitor of GLP-1 intracellular metabolism would be a DPP-4 inhibitor such as sitagliptin, saxagliptin, linagliptin, at it would be obvious to one skilled in the art that any DPP-4 inhibitor would be within the scope of the invention. Any inhibitor of Insulin intracellular metabolism would be within the scope of the invention. In a preferred embodiment, the inhibitor of Insulin intracellular metabolism would be an inhibitor of insulin degradation enzyme (IDE), which would ordinarily be an inhibitor of a Zinc Metalloproteinase, as previously disclosed by Leissring in 2010 (17)

Leissring discloses novel peptide hydroxamic acid inhibitors of IDE. The resulting compounds are approximately 10(6) times more potent than existing inhibitors, non-toxic, and surprisingly selective for IDE vis-a-vis conventional zinc-metalloproteases. These IDE inhibitors potentiate insulin signaling by a mechanism involving reduced catabolism of internalized insulin. The distinctive structure of1DE′s active site, and the mode of action of our inhibitors, suggests that it may be possible to develop inhibitors that cross-react minimally with conventional zinc-metalloproteases. One specific form of the IDE inhibitor disclosed by this group and suitable for use in the present invention is ML345.(18)

In another embodiment, said peptide loaded cholestosome contains insulin glargine and lixisenatide and optionally an inhibitor(s) of metabolism inside body cells. A general inhibitor of enzyme degradation inside cells and therefore suitable for inclusion in this formulation is Soybean Trypsin Inhibitor.

In another embodiment, said peptide loaded cholestosome contains Dulaglutide

In another embodiment, said, peptide loaded cholestosome contains Semaglutide

In another embodiment, said peptide loaded cholestosome contains liraglutide

In one embodiment, the amount of insulin utilized in methods and compositions of the present invention is 0.5-3 units (u)/kg in humans. In one embodiment, the units used to measure insulin in methods and compositions of the present invention are USP insulin Units. In one embodiment, the units used to measure insulin are milligrams. In another embodiment, one USP insulin Unit is equivalent to 34,7 mg, insulin.

In another embodiment, the amount of insulin for a human patient is 0.1-1.0 u/kg. In another embodiment, the amount is 0.2-1.0 u/kg. In another embodiment, the amount is 0.3-1.0 u/kg. In another embodiment, the amount is 0.5-1.0 u/kg. In another embodiment, the amount is 0.1-2.0 u/kg. In another embodiment, the amount is 0.2-2.0 tag. In another embodiment, the amount is 0.3-2.0 u/kg. In another embodiment, the amount is 0.5-2.0 u/kg. In another embodiment, the amount is 0.7-2.0 tag. In another embodiment, the amount is 1.0-2.0 u/kg. In another embodiment, the amount is 1.2-2.0 u/kg. In another embodiment, the amount is 1.01.2 in another embodiment, the amount is 1.0-1.5 u/kg. In another embodiment, the amount is 1.0-2.5 u/kg. In another embodiment, the amount is 1.0-3.0 u/kg. In another embodiment, the amount is 2.0-3.0 u/kg. In another embodiment, the amount is 1.0-5.0 u/kg. In another embodiment, the amount is 2.0-5.0 u/kg, in another embodiment, the amount is 3.0-5.0 u/kg.

In another embodiment, the present invention provides a method for treating diabetes mellitus in a subject, comprising administering orally to the subject a pharmaceutical composition comprising an insulin and optionally other peptides and optionally one or more inhibitors of intracellular metabolism of said substance, thereby treating diabetes mellitus.

In certain embodiments, the cargo loaded vesicle is placed in a capsule and the capsule surface is further enterically coated to prevent degradation of the pharmaceutical composition in the stomach acid of the gastrointestinal tract.

In certain embodiments, the surface layer of the peptide-loaded cholestosome remains intact at a pH range of between about 2 to about 14,

In other embodiments, the cargo-loaded cholestosome is a unilamellar vesicle having a diameter of about 250 nm up to more than 10,000 nm (10 micrometers), about 10 nm to about 1000 nm, often about 50 nm to about 750 nm, about 1000 to about 2500 nm, about 200 to about 300 nm, depending upon whether the material is subjected to an extrusion step or is used un-extruded. Accordingly, it is noted that larger cholestosomes are used when the active molecule is larger and small cholestosomes are used when the active molecule is smaller.

In one embodiment, the protein is a recombinant protein. In one embodiment, the protein is an insulin, in another embodiment, the protein is a glucagon. In another embodiment, the protein is an interferon gamma. In another embodiment, the protein is an interferon alpha. In another embodiment, the protein is an interferon beta. In another embodiment, the protein is an erythropoietin. In another embodiment, the protein is granulocyte colony stimulating factor (G-CSF). In another embodiment, the protein is any other protein known in the art.

In another embodiment, the protein is a growth hormone. In one embodiment, the growth hormone is somatotropin. In another embodiment, the growth hormone is insulin Growth Factor-I (IGF-1). In another embodiment, the growth hormone is any other growth hormone known in the art.

In another embodiment, the protein has a molecular weight (MW) of 1-50 kilodaltons (kDa). In another embodiment, the MW is 1-450 kDa. In another embodiment, the MW is 1-400 kDa. In another embodiment, the MW is 1-350 kDa. In another embodiment, the MW is 1-300 kDa in another embodiment, the MW is 1-250 kDa. In another embodiment, the MW is 1-200 kDa. In another embodiment, the MW is 10-50 kDa. In another embodiment, the MW is 15-50 kDa. In another embodiment, the MW is 20-50 kDa. In another embodiment, the MW is 25-50 kDa. In another embodiment, the MW is 30-50 kDa. In another embodiment, the MW is 35-50 kDa. In another embodiment, the MW is 1-100 kDa. In another embodiment, the MW is 1-90 kDa. In another embodiment, the MW is 1-80 kDa. In another embodiment, the MW is 1-70 kDa. In another embodiment, the MW is 1-60 kDa. In another embodiment, the MW is 10-100 kDa. In another embodiment, the MW is 15-100 kDa. In another embodiment, the MW is 20-100 kDa. In another embodiment, the MW is 25-100 kDa. In another embodiment, the MW is 30-100 kDa. In another embodiment, the MW is 10-80 kDa. In another embodiment, the MW is 15-80 kDa. In another embodiment, the MW is 20-80 kDa. In another embodiment, the MW is 25-80 kDa. In another embodiment, the MW is 30-80 kDa. Each possibility represents a separate embodiment of the present invention.

In another embodiment, the MW is less than 20 kDa. In another embodiment, the MW is less than 25 kDa. Another embodiment, the MW is less than 30 kDa. In another embodiment, the MW is less than 35 kDa. In another embodiment, the MW is less than 40 kDa. In another embodiment, the MW is less than 45 kDa. In another embodiment, the MW is less than 50 kDa. In another embodiment, the MW is less than 55 kDa. In another embodiment, the MW is less than 60 kDa. In another embodiment, the MW is less than 65 kDa. In another embodiment, the MW is less than 70 kDa. In another embodiment, the MW is less than 75 kDa. In another embodiment, the MW is less than 80 kDa. In another embodiment, the MW is less than 85 kDa. In another embodiment, the MW is less than 90 kDa. In another embodiment, the MW is less than 95 kDa. In another embodiment, the MW is less than 100 kDa.

The molecular weights of some of the proteins mentioned above are as follows: insulin-6 kilodalton (kDa); glucagon-3.5 kDa; interferon, 28 kDa, growth hormone-21.547 kDa; human serum albumin-69 kDa; erythropoietin-34 kDa, G-CSF-30-34 kDa; trastuzumab 150 kDa; Catalase 280 kDa. Thus, in one embodiment, the molecular weight of these proteins is appropriate for administration by methods of the present invention.

In another embodiment, methods and compositions of the present invention are used to administer a human serum albumin. Human serum albumin is not, in one embodiment, considered to be a pharmaceutically-active component; however, it can be used in the context of the present invention as a therapeutically-beneficial carrier for an active component that may bind preferentially to albumin.

Each type of protein represents a separate embodiment of the present invention.

### Pathway for Loaded Cholesteryl Ester Vesicles Used in the Present Invention

In one embodiment, the invention provides a cholesteryl ester vesicle composition comprising an active pharmaceutical agent, a molecule which in preferred embodiments is a macromolecule, even more often a peptide (“peptide-loaded cholestosome”), for example, a pharmaceutically-active agent (which term includes therapeutic and diagnostic agents) which is encapsulated by a surface layer of neutral charge comprising one or more cholesteryl esters produced from cholesterol and one or more saturated or unsaturated fatty acids. The cholestosomes according to the present invention encapsulate one or more different active pharmaceutical agent molecules of wide variety of size and weight, especially pharmaceutical active molecules which are difficult to deliver by the oral route using prior art methods, including liposomes, pegylation, dendrimers, cationic nanoparticles and the like.

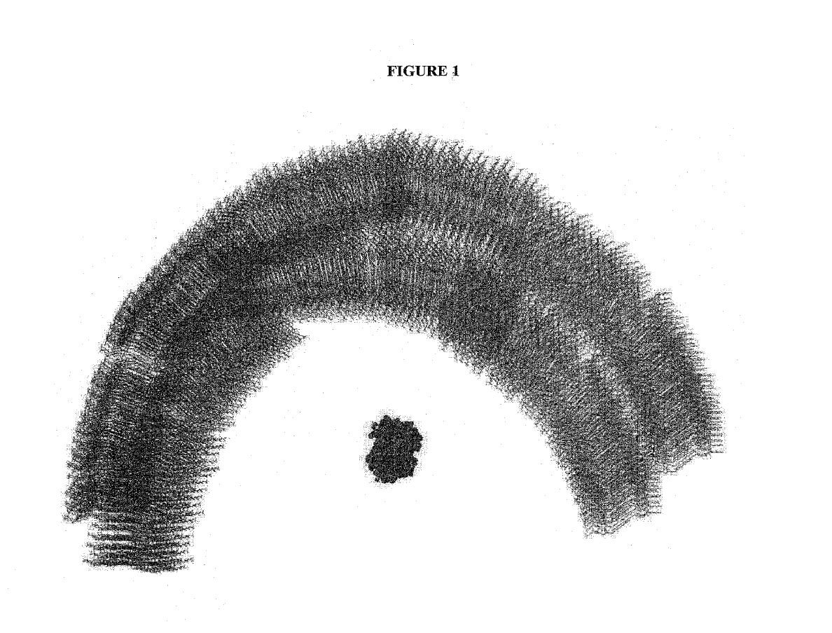

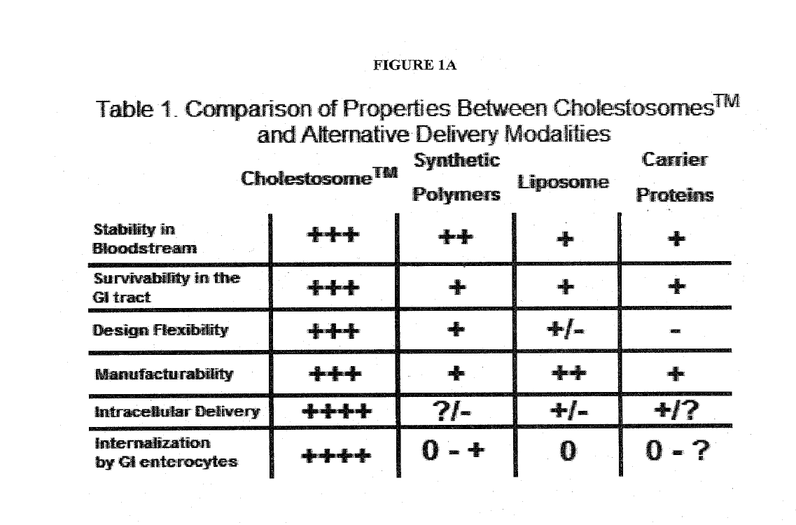

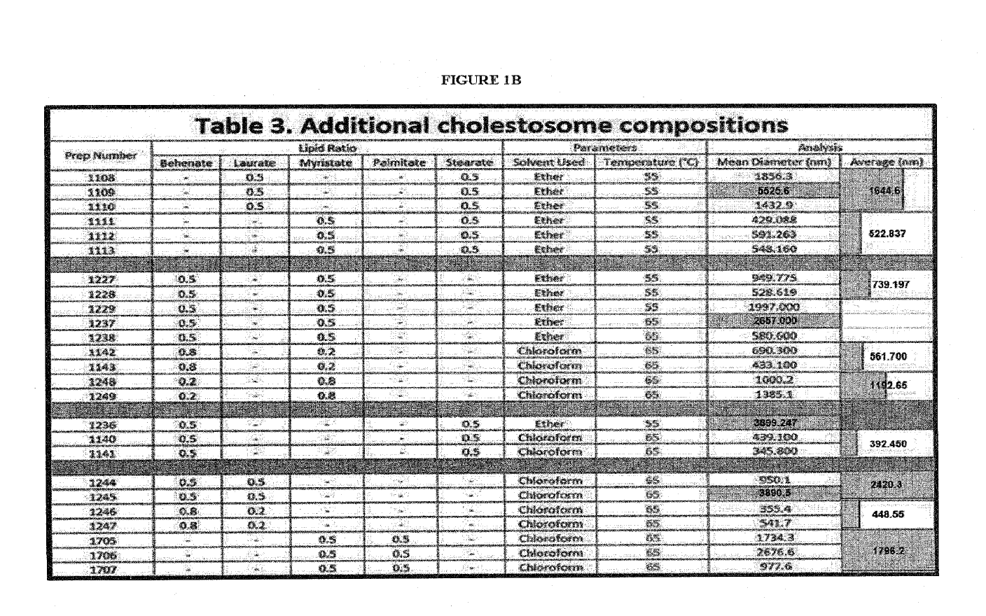

### FIG. 1A, Table 1: A Comparative Analysis of the Properties of Cholesteryl Ester Vesicles and Alternative Delivery Technologies is Disclosed

### Oral Absorption and Intracellular Delivery Using Cholestosomes

Pursuant to the present invention, said peptide-loaded cholestosomes, after administration to a mammal, a patient or a subject, are of specific, composition to enable intact incorporation into chylomicrons (the specific steps in this process are carried out in the body uniquely by intestinal enterocytes) to produce a chylomicron containing said cholestosome vesicle and its intact payload. Said cargo loaded chylomicrons are then released by gastrointestinal enterocytes into the lymphatics and subsequently into blood by entry of lymph into the thoracic duct. After traveling thru the heart, said chylomicrons loaded with vesicles are accessible to all cells receiving said arterial blood supply, although only cells expressing a chylomicron docking receptor will have access to said contents. After receptor mediated docking of the chylomicrons with these cells, the cholestosome and its payload is delivered intact into said cells, wherein said cholestosome is disassembled by action of cholesteryl hydrolase to break the bond between cholesterol and the fatty acid, thereby releasing the encapsulated active molecules inside the membrane into the cytoplasm of said cells. The impact of the present invention is to directly deliver orally administered active molecules inside cells to effect therapy or diagnosis.

Pharmaceutical compositions and oral methods of treatment of the invention, when encapsulated with said cholesteryl esters, enable chylomicron-targeted intracellular delivery of a variety of active ingredients that are, in an unprotected state, ineffective due to degradation in vivo. For example, the invention enables effective delivery of macromolecules useful in the treatment of inflammation-associated metabolic disorders as defined herein, vaccines to specific sites in the body, genetic materials inside cells where they act in the ribosomes and nuclei, and even topical delivery on the skin with the potential for passage of the skin barrier in some specific embodiments. Other methods of treating disease states and/or conditions using compositions according to the present invention are also disclosed herein. Virtually any pharmaceutically active molecule can be delivered efficiently into target cells of a patient or subject in the manner of the present invention, and the result in effective therapy is unmatched by delivery methods of the prior art. Methods of treating disease states and conditions by administering compositions according to the present invention to a patient in need represent additional embodiments according to the present invention. Effective dosages of compositions for methods of treatment embodiments according to the present invention may range from as little as one microgram or less up to one gram or more per day. Other effective dosages will depend on the size and age of the patient or subject, the general health of the patient and the potency of the molecule among a number of other facts. Dosages contemplated within the range of less than about 0.0001 mg/kg/day up to about 100 mg/kg/day or more with ranges of about 0.001 mg/kg/day to about 25 mg/kg/day being more often utilized.

In certain embodiments, the pharmaceutical composition is a unilamellar vesicle in which between about 10% to about 98%, about 20% to about 96%, often about 50% to about 96%, often about 90% to about 96% of the vesicle's total weight is the weight of the molecule or said pharmaceutically-active agent.

In the present invention, the mass ratio of the active molecule (which preferably includes a pharmaceutically-active agent), to one or more cholesteryl esters is between about 4:96 to about 96:4, about 10:90 to about 96:4, often about 10:90 to about 96:4, often about 20:80 to about 90:10, about 20:80 to about 50:50, about 50:50 to about 96:4, about 90:10 to about 96:4.

In the present invention, the pharmaceutical composition is not altered when incorporated into said cholestosome vesicle, and upon release by cholesteryl ester hydrolase inside said body cells, said pharmaceutical composition has the same activity and is identical to the Active Agent,

In another embodiment, an interdigitated alternating alkyl chain model of cholesteryl ester inter-digitation is used to maximize the mass ratio of the active molecule, including a pharmaceutically-active agent to one or more cholesteryl esters by selecting the one or more cholesteryl esters based on pharmaceutically-active agent-cholesteryl ester functional group interaction. Example 2, infra describes formulation criteria which ensure optimal pharmaceutically-active agent-cholesteryl ester functional group interaction.

In another embodiment, the pharmaceutical composition is a cholestosome vesicle made by a process comprising reacting one or more of the cholesteryl esters in diethyl ether, removing the resultant organic phase under vacuum and introducing an aqueous phase which contains a high concentration of the peptide to be encapsulated.

In still another embodiment, cholesteryl esters are selected based on their reactivity with cholesterol transporters on the surface of duodenal enterocytes and ability to remain intact in enterocytes until incorporation into chylomicrons.

In embodiments according to the invention, the cholesteryl ester is obtained by esterifying cholesterol with a C₆ to C₃₆ saturated or unsaturated fatty acid, often a C₅ to C₂₆ fatty acid, even more often a C₈-C₂₂ fatty acid or a C₈-C₁₄ fatty acid. In certain preferred embodiments, the cholesteryl esters are selected from the group consisting of cholesteryl myristate, cholesteryl laurate, cholesteryl dodeconate, cholesteryl palmitate, cholesteryl arachidonate, cholesteryl behenate, cholesteryl linoleate, cholesteryl linolenate, cholesteryl oleate and cholesteryl stearate.

Cholestosomes pursuant to the present invention are unique among delivery systems for molecules. For the first time, the inventors have successfully disguised proteins and other molecules and chemical compounds as fatty acids, which are dietary lipids commonly known in the art as food. Most specifically, the chosen materials for oral uptake are dietary cholesteryl esters. Surprisingly the cholesteryl esters provide a unique cholesteryl ester vesicle having the following properties that differentiate cholestosome encapsulated products (especially macromolecules which cannot otherwise be delivered to patients with any real measure of success) over liposomes or any other vesicle:
1. All component materials of the delivery means and system are common dietary ingredients, and total dosage of these substances per day in most applications will be less than from food.
2. Working temperature for encapsulation in cholestosomes is often 35-45 degrees centigrade, which is an optimal temperature for the stability of peptides and proteins in their body circulating forms.
3. Said Delivery means will offer all favorable aspects without concern for molecular size, charge, binding or degradation pathways
4. Cholestosome encapsulated proteins show complete passage of Caco2 enterocyte barrier, and are incorporated intact into chylomicrons
5. Bypass of the liver and associated first pass clearance pathways
6. Cholestosomes and the chylomicrons that contain them, provide protection for molecules as they pass cell membranes from oral intake all the way to intracellular uptake
7. Docking with cells; Quantitative intracellular loading; Complete passage of cellular membrane barrier
8, Unpacking of encapsulated contents in cytoplasm by cholesteryl ester hydrolases, an endogenous pathway.
9. Robust intracellular concentration of payload molecules at intracellular sites, yet cholestosomes do not use endosome uptake pathways
10. Cholestosomes and their encapsulated contents are distributed into all cells when incorporated into native formed chylomicrons

While some deliver systems may achieve one or a small number of these 10 features, there is no other delivery system that can achieve this wide array of favorable properties, especially when the delivery system enables oral use of heretofore unabsorbed proteins, and does not alter the payload molecules and can be employed for essentially any molecule or chemical compound. Cholestosomes are the first intracellular delivery system that can be applied to any molecule. In fact, cholestosomes are at least as efficient with macromolecules, especially including proteins, peptides, polynucleotides (RNA and DNA, including, for example, naked DNA, plasmid DNA, interfering RNA or “RNAi”, including small interfering RNA or “siRNA”, small hairpin “shRNA”, bifunctional shRNA, microRNA and various oligonueleotides of DNA and RNA, among others) and macromolecular antibiotics, among others, as they are with small molecules.

Because of the unique mechanism of delivering active molecules to a target within cells of a patient or subject, cargo loaded cholestosomes according to the present invention are capable of delivering cargo (i.e., active macromolecules) within cells of a patient or subject to whom the present compositions are administered (preferably, orally) to a concentration of at least 2 times that which is provided in the absence of cholestosomes (i.e., by conventional pharmaceutical delivery means, including delivery in liposomes). In most embodiments, the present invention delivers active molecules within cells to a concentration at least 10 times, 25 times, 50 times, 100 times, 250 times, 500 times and 1,000 times or more that which is provided (delivered into cells) in the absence of cholestosomes.

As cholestosomes are novel in relation to prior art for molecule and chemical compound delivery, the inventors provide detailed comparison information to the reader in order to point out why prior art does not disclose any similar system,

Following these comparisons, Non-limiting examples will be provide

Cholesterol has vital structural roles in membranes and in lipid metabolism in general, it is a biosynthetic precursor of bile acids, vitamin D and steroid hormones (glucocorticoids, oestrogens, progesterones, androgens and aldosterone). In addition, it contributes to the development and working of the central nervous system, and it has major functions in signal transduction and sperm development. It is found in, covalent linkage to specific membrane proteins or proteolipids (hedgehog' proteins), which have vital functions in embryonic development.

Cholesterol esters, preferably with long-chain fatty acids linked to the hydroxyl group (often prepared from fatty acids containing at least eight up to 26 carbon atoms), are much less polar than free cholesterol and appear to be the preferred form for transport. In plasma and as a biologically inert storage (de-toxification) form. They do not contribute to biological membranes but are packed into intracellular lipid particles.

Cholesterol ester hydrolases in animals liberate cholesterol and free fatty acids front the ester form, when required for membrane and lipoprotein formation. They also provide cholesterol for hormone synthesis in adrenal cells. Many cholesterol ester hydrolases have been identified, including a carboxyl ester hydrolase, a lysosomal acid cholesterol ester lipase, hormone-sensitive lipase and hepatic cytosolic cholesterol ester hydrolase. These are located in many different tissues and organelles and have multiple functions.

The inventors disclose a novel delivery technology which encapsulates molecules in a cholesteryl ester particle called a cholestosome, and after this particle is orally absorbed by cells of the intestine, it is placed into chylomicrons for delivery to all body cells via lymphatic transport. After this vesicle is taken up into cells from the chylomicron transport particle, the cholesterol ester hydrolases unpack the particle and liberate the molecule at the intracellular site.

Relevant background information regarding the structure of the cholestosomes in this application is found in United States Patent Application Document No. 20070225264, filed Mar. 20, 2007 and entitled “Drug Delivery Means”.

Principles of interdigitation as used herein are known to those of ordinary skill in the art. See e.g. Yeagle, The Structure of Biological Membranes (CRC Press 2010).

“Chylomicrons” are very large, heterogeneous, lipid-rich particles ranging in diameter from about 750 to 40,000 nm. They are formed in the enterocytes of the GI tract and function to transport dietary fat and fat-soluble vitamins to cells via circulation in the bloodstream. The size heterogeneity of the secreted chylomicron particles depends on the rate of fat absorption, type and amount of fat absorbed. When cholestosomes are very large, the resulting chylomicrons that incorporate these large cholestosomes can be larger as well.

“Cholestosomes” are stable in the adverse conditions of the GI tract, possess greater design flexibility, and exhibit greater encapsulation efficiency fix a wide variety of molecules, and have advantages of easier manufacturability. These favorable cholestosome properties are emphasized in FIG. 1A, Table 1, which compares delivery systems. The structural differences between cholestosomes and liposomes confer on cholestosomes different physical and chemical properties and therefore permit them superiority in desired properties and functions. For example, cholestosomes have been shown to be stable over a wide pH range from 2 to 13. In contrast, according to a 2005 review article in the journal of Molecular structure describing liposomes, “owing to the small resistance of liposomes to gastric juice (pH 1.9), enzymes of the alimentary canal and bile acids in the intestine (pH 8) their application per os is useless.” Cholestosomes resist pH degradation and therefore have the potential to be used as a primary means for oral delivery of molecules, a particularly novel aspect of the present invention.

A cargo-loaded cholestosome” refers to, cholestosome which has encapsulated a pharmaceutically active agent, in particular a macromolecule and contains the agent principally, although not necessarily exclusively, in the core of the cholestosome vesicle.

Secondly, the structural features based on the interaction of the cholesteryl esters confers electrostatic surface properties which are calculated to be similar to PEG surfaces which liposomes use to confer enhanced time in the blood system. This confers upon the drug or molecule contained within the cholestosome a longer residence time in the body, normally an advantage of a drug delivery system, but not necessarily an advantage if the molecule cannot he released from the drug delivery vesicle.

The evidence for this is the Zeta potential measurements showing cholestosomes with a neutral surface in one formulation cholestosomes have a measured Zeta potential of −14, which is typical of a neutral charge to cholestosomes alone in their unloaded form. Neutral charge boundaries for Zeta potential means having a Zeta potential of about −40 to +40, often about −20 to about +20, often about −40 to +10, −5 to +5 or approximately 0. The push for neutral surface charge leads to the use of PEG is used in other types of formulations. Cholestosomes approximate the neutral surfaces of. PEG in certain comparisons of embodiments among the various inventions.

Structural modifications of cholestosomes are based on modification of mole ratios of the esters which result in different interior and exterior surface properties and in cholestosomes those properties are not defined by an organization based on hydrophilic/hydrophobic sequestration (as in liposomes and other prior art delivery means: and therefore are more easily defined and manufactured, (Evidence of size as a result; of sonication, often temperature, often pH (aqueous solutions of neutral pH have different charges on the molecules for encapsulation which may affect their ability to define the size oaf the lipid vesicles)). All of these beneficial properties are summarized and compared with those of other delivery systems in FIG. 1A, Table 1 below.

As shown in FIG. 1A, Table 1, below. Upon comparison of properties between Cholestosomes and alternative delivery modalities evidences that cholestosomes are superior or at least equal in all categories. One particularly important aspect of this comparison is that nearly any molecule can be encapsulated into a cholestosome without altering the molecule itself. This feature is not shared with other delivery systems, which tend to be specific to the molecule itself. Design flexibility is an advantageous property for a drug delivery system, clearly evident in the present invention.

FIG. 1A, Table 1
Comparison of properties of Cholestosomes
with other common means of drug delivery
Table 1. Comparison of Properties Between
Cholestosomes ™ and Alternative Delivery
Modalities

| | | Synthetic | | Carrier |
|---|---|---|---|---|
| | Cholestosomes ™ | Polymers | Liposome | Proteins |
| Stability in the | +++ | ++ | + | + |
| Bloodstream | | | | |
| Survivability in the | +++ | + | + | + |
| GI tract | | | | |
| Design Flexibility | +++ | + | +/− | − |
| Manufacturability | +++ | + | ++ | + |
| Intracellular | ++++ | ?/− | +/− | +/? |
| Delivery | | | | |
| Internalization | ++++ | 0-+ | 0 | 0-? |
| by GI enterocytes | | | | |

In FIG. 1A, Table 1 above, synthetic polymers refer generally to techniques such as PEGylation. Carrier proteins refers to attached biological molecules such as viral vectors. Both PEGylation and Carrier proteins constructs are given intravenously, and like liposomes, are not absorbed if given orally, primarily because they are degraded in the GI tract

### Peptide Oral Absorption and Favorable Associated Properties

While not being limited by way of theory, the present invention enables oral delivery of a formulation means that encapsulates a molecule, in preferred embodiments one or more peptides or a protein into a cholesteryl ester vesicle which enters GI enterocytes through molecular recognition, is ingested, incorporates into a chylomicron, thereby fully protecting the integrity of the molecule during its passage through the enterocytes of the gastrointestinal tract, into chylomicrons before entering the lymphatic system, in the blood, and across the membranes of body cells. Disclosed formulations of the invention herein are stable and do not release an active ingredient until it has been taken into the cells of the body. Features of commercial embodiments of this invention, called cholestosomes, include the following:
1) complete passage of Caco2 enterocyte barrier by specific fatty acid transporter mediated uptake without endosomes, followed by insertion of intact vesicle into chylomicrons;
2) complete passage of non-enterocyte cellular membrane barrier without endosomes but with release from vesicle by cholesteryl ester hydrolase
3) a method of intracellular delivery similar to the Trojan Horse, where the delivery method largely avoids endosome uptake and yields a payload that is free in cytoplasm;
4) high oral bioavailability and delivery and uptake into body cells, that is relatively independent of active molecule size, charge, binding or degradation pathways, whereby the surface of the peptide-loaded cholestosome has a neutral charge in some applications.
5) active ingredients circulate in lymphatics around the liver an oral deliver method that avoids the so-called first pass hepatic uptake and yet does eventually reach the liver: and
6) Peptide delivery into cells is facilitated by apolipoprotein attachments to surfaces of chylomicrons, said chylomicrons capable of docking with cells and intracellular loading, followed by unpacking of encapsulated molecules in cytoplasm under the action of cholesteryl ester hydrolases.

Accordingly, peptide-loaded cholestosomes according to the present invention are the only viable means of delivering one or more peptides (i.e., active molecules in preferred embodiments) to a concentration within cells of a patient or subject to whom the present compositions are administered (preferably, orally) of at least 2 times that which is provided in the absence of administration in cholestosomes (i.e., by conventional pharmaceutical delivery means, including delivery in liposomes). In most embodiments, the present invention after oral use delivers active molecules within cells to a concentration at least 10 times, 25 times, 50 times, 100 times, 250 times, 500 times and 1,000 times or more that which is provided in the absence of cholestosomes. The bioavailability of preferred compositions according to the present invention ranges from 50% to about 100% (e.g., 99.9+%), 55% to 99.9%, 60% to 99.5%, 65% to 99%, 70% to 98%, 75% to 95%, 50% to 95%. etc, which is calculated on the basis of oral to parenteral AUC (area under the curve). Thus, the present compositions afford unexpectedly high bioavailability especially from oral compositions.

Thus, the present invention provides a means to encapsulate molecules of a variety of size and molecular weight which heretofore could not be accommodated (itself an unexpected result) and regardless of size, the present compositions are capable of delivering’. active molecules to targets in cells at concentrations much higher levels than the prior aft. Accordingly, the dosages of active compounds which can be administered to patients according to the present invention is often less than using traditional compositions and as little as 1% to 50%, often 5% to 50% or 10% to 25% of the dosage required usinu, standard orally administered compositions which are not based upon the present invention.

### Bioavailability After Oral Use As Compared to Injection

In pharmacokinetics and in particular pharmacokinetic comparisons, bioavailability is a means of quantifying (usually oral) absorption. Bioavailability is the fraction of an administered dose of unchanged drug that reaches the systemic circulation, one of the principal pharmacokinetic properties of drugs. Usually the pharmacokinetic comparison is represented as the Area Under the Curve (AUC) after an oral dose to the AUC after the same dose intravenously (IV). By definition, when a medication is administered intravenously, its bioavailability is 100%. However, when a medication is administered via other routes (such as orally), its bioavailability generally decreases (due to incomplete absorption and lor first-pass metabolism) or it may vary from patient to patient. Bioavailability is one of the essential tools m pharmacokinetics, as bioavailability must be considered when calculating dosages for non-intravenous routes of administration.

In the present application, the inventors will show that oral cholestosome encapsulated insulin has surprisingly higher bioavailability than previously seen with any oral delivery system. In additional experiments, the cells of organs and tissues contain many fold higher FITC concentrations when given FITC insulin cholestosomes,

### Cholestosomes Slowly Enter Cells On Their Own

Intravenously administered, cholestosomes not in chylomicrons would not dock with cells, as they are lacking the surface apolipoproteins which are necessary for docking a chylomicron with a cell in need of lipids. However, cell membranes do appear to have transporters for fatty acids, which clearly take in cholestosomes (see MCF-7 cell data). Thus, parenteral administration of cholestosomes does allow some intracellular uptake of certain cholesteryl ester vesicles with affinity for the transporters, intracellular uptake is much greater if these same cholestosomes are given orally and cholestosomes are presented to cells encased in chylomicrons.

Cholestosomes Themselves Enter Into Body Cells with Payload Intact

As can be appreciated from the images of MCF-7 cells in Example 3, there is nearly always an appreciable cellular uptake of molecules from cholestosomes alone, as there are surface transporters on most body cells that recognize the cholesteryl esters chosen as components of the vesicle.

Docking with Body Cells because of APO-B Incorporation into Chylomicrons

Intracellular delivery of macromolecules encapsulated within cholestosomes and incorporated within chylomicrons is accomplished when the chylomicrons containing the cargo-loaded cholestosome containing an active molecule payload dock with cells in need of cholesterol and triglycerides and transfer said components including said cholestosomes into cells without requiring any secondary encapsulation in an endosome, Endosomal formation with a cholestosome encapsulated macromolecule is still possible in the view of the inventors, but it does not appear to be the usual process that is ongoing during ingestion of cholesteryl ester cholestosomes.

### Chylomicrons Amplify Cell Uptake Over Cholestosomes Alone

Cholestosomes clearly enable greater amounts cell uptake after oral absorption because they are first taken into chylomicrons. Chylomicrons then selectively deliver lipids to cells which are in need thereof. Cells in need express a docking site protein which then can link to the APO-B on the surface of the chylomicron, thus effecting docking and release from the chylomicron into the cytoplasm of the cell. Furthermore, the chylomicrons that are formed from cholestosomes have. Apolipoprotein recognition properties on the surface that reaches every cell. As chylomicrons contact cells, they dock with cells that are expressing surface proteins and thereby requesting transport of lipids including triglycerides and cholesteryl esters. After lipases are disgorged from the cell, said lipids such as triglycerides and the cholestosomes are taken into the cell including their encapsulated payloads,

By Contrast, when liposomes are injected into the blood they would not be expected to dock with cells, as they are lacking Apo E constituents for docking with cells seeking lipids. Liposomes serve to create a prolonged plasma release characteristic to molecules in drug delivery. Furthermore, in the favorable occasion where the drug encapsulated within a liposome delayed release system does enter the cell, then it would be expected that there is intracellular delivery of payload because of the property of the drug after it is freed from the carrying liposome.

### Unpacking of Cholestosomes Inside Cells, Cellular Responses and Exocytosis Pathways

Here we discuss cholesterol ester hydrolase as applied to cholesteryl esters after they pass the cell membrane, as the action of this enzyme is to separate the cholesterol nucleus from the carrier fatty acid. When this enzyme acts on the cholesteryl ester, the macromolecule is freed from the cholestosome. Once freed from the delivery system, the next step is pharmaceutical action inside the cells.

Free peptides inside cells may be metabolized, so it becomes necessary to inhibit this metabolism if there is to be any peptide left for exocytosis and continued circulation. For certain molecules in the practice of the invention, such as insulin by example, it will be necessary for the skilled artisan to include an inhibitor of metabolism in the cholestosome vesicle to prevent excessive intracellular metabolism of this substance.

By way of example, if the goal is to create extracellular action, then intracellular metabolism must be stopped or slowed, so the cell chooses exocytosis of the free molecule. This is particularly pertinent to insulin and its intracellular metabolism, since that is the clearance pathway, and the cells rapidly clear insulin by metabolism.

We performed experiments to show that protease inhibitors can stop intracellular metabolism of insulin and thus favor exocytosis of free insulin.

### Formulation of Peptide Loaded Cholestosomes

Cholestosomes are formed in several stages, tint by dissolution of, the pair of chosen cholesteryl esters in organic solvent such as ether, then removal of the organic solvent, and next there is addition of aqueous component which contains the molecule to be encapsulated, with sonication to form the unilamellar membranes and generate the hydrophilic relatively uncharged hollow pocket around molecules in aqueous.

All formation stages are carried out with a water bath at a critical specified temperature which is based on the lowest melt temperature of the esters. Working temperature is a primary condition for selection of cholesteryl ester pairs, as the melt temperature of the, chosen pairs of esters must be equal to or lower than the temperature that will degrade the molecule chosen for encapsulation. With the temperature limits in mind, the cholesteryl ester pairs must be chosen to form a bilayer membrane at temperatures below 45° C. degrees Centigrade, winch is a basis for choice of cholesteryl myristate and cholesteryl titrate for many of the examples disclosed as part of the present invention.

FIG. 1 depicts a three-dimensional model of a cholesteryl laurate/cholesteryl myristate (1:1 molar concentration) cholestosome. Cholestosomes can have a wide range of sizes. Active ingredient loading can he determined through calculations such as those shown in the preferred examples.

Once there is the addition of the aqueous molecule or construct, the mixture is sonicated until there is a cloudy solution formed, thereby minimizing waste from un-dissolved esters, with sonication providing energy for unilamellar vesicle formation. The aqueous component is also maintained at the target temperature prior to its addition, and as stated previously for most peptides, proteins and genes, the highest temperature that can be tolerated is only about 45-50° C. Under certain conditions, the inventors have surprisingly found that insulin will remain stable up to 55° C.

The solution is then filtered and the filtrate is saved for extrusion for size conformity. The sample is then stored in the refrigerator, where it remains stable for more than 30 days.

The newly encapsulated molecule is surrounded by the unilamellar cholesteryl ester vesicle and inside the hollow pocket the encapsulated molecule is protected from contact with the harsh environment of the GI tract and is held away from enzymes and the cells of the immune system. The molecule inside remains unchanged. Accordingly, providing cargo-loaded cholestosomes pursuant to the present invention is a facile, routine undertaking.

### Cholesteryl Ester Vesicle Construction (FIG. 1)

The outer membrane of Cholestosomes consists of cholesteryl esters arranged to form a lipid vesicle based upon cholesteryl esters, generally in the case where the plurality of cholesteryl esters have the same or similar molecular length, so as to form a uniform capsule around a macromolecule encapsulated by said cholestosome. The cholesteryl esters may be of different lengths as long as they are co-soluble, which will permit them to aggregate together to form a vesicle with a rather large hollow core in relation to the total size of the lipid vesicle. In fact, some configurations have the core displacement well beyond 80 percent of the entire vesicle, which affords beneficial high loading of water soluble molecules such as insulin.

This is based on the ability of differential mole fractions of different esters being able to co-exist and aggregate in a minimum energy conformation in which the vesicle formation is determined by the nature of the cholesteryl esters and their relative mole fractions.

An illustration of an assembling vesicle around a molecule, in this case insulin is found in FIG. 1. Here, the chains are configured in a circular format so as to form a hollow center which has a neutral or mildly negative charge (Zeta potential measurements are made to define this property, as will be shown in the examples for each formulation disclosed. Cholestosomes alone have a Zeta Potential reading of −14).

With Insulin in the cholestosome, its Zeta Potential goes additionally negative. Cholestosome encapsulated formulations do not have highly positive charges, in contrast to Liposomes, where the Zeta potential could range as high as +76 in some experiments.

In this and other examples, the cholesteryl esters may be of different lengths as long as they are co-soluble, which will permit them to aggregate together to form a unilamellar vesicle. This is based on the ability of differential mole fractions of different esters being able to co-exist and aggregate in a minimum energy conformation in which the hollow core of the vesicle is determined by the nature of the cholesteryl esters and their relative mole fractions.

As the assembly of cholestosomes are considered and with reference to the 3D diagram as FIG. 1. consider first the assumption that the interior, and exterior of the cholestosome matrix are the same structurally in that the sterol nuceli point both into the cavity and out to the surface.

One feature subject to change by the artisan, by choice of cholesteryl esters, is the length of the ester tails. Having a shorter tail length brings the sterol nuclei closer to each other and lessens the hydrophobic nature of the vesicle (due to chain length). This may have an enhancing impact on the hydrophilic character of cholestosome. This can, be modeled and examples are presented in FIGS. 2 to 6.

Furthermore, assuming the same packing of the inner core of the lipid vesicle irrespective of chain length, shorter chains around a larger molecule would increase the mass to mass ratio of the molecule to the lipid. Clearly, larger molecules need larger internal cores, and hence ester chain length is important for the construction of larger cholestosomes to accommodate larger molecules such as monoclonal antibodies.

Balanced against these considerations is the impact of ester chain length on the relative hydrophilicity of the inner core. Longer ester chains increase the hydrophobic character and allow for packing of a more hydrophobic molecule into the core.

There is also the issue of interactions of matrix cholesteryl esters interacting with solvents.. Aqueous solvent combinations including ethanol may help in the encapsulation process overall, and increase the amount encapsulated at a fixed ratio of cholesteryl esters. This is the impact of charge of the construct and charge of the inner core of the cholestosome

For example, in crystal structures of oxysterols, changing the solvent ratio by including, an alcohol such as ethanol helps bring the oxygen molecules closer to each other, which may help the esters orient in a cholestosome and also'help bring the molecules into the core of the cholestosome vesicle more readily.

In modeling of these interactions, the inventors can examine models of cholesteryl ester matrix structures and predict which esters are the best choices for specific molecules or drugs. A systematic approach is possible when the interactions between esters, charge, solvents and molecule are considered simultaneously.

Temperature range during production of cholestosome vesicles is 35° C. to 45° C. when working with most of the cholesteryl esters in Table 2. Temperature is held constant (+/−5C) throughout the preparation of the vesicles. Temperature is kept below the melt temperature of any of the individual esters. By way of example, for the preparation of cholestosomes using myristate/laurate, temperature is optimally held at 40° C. +1/−5 C. Addition of small amounts of surface active agents or charged ions between to the mixture components prior to sonication increase overall yield of cholestosomes and facilitate the production of more uniform particles, as will be disclosed.

Sonication times range from 10 min to 30 minutes. This time is presented as a range, in that centrifuge time is a variable. Optimal sonication time depends on the ability to find the optimal sonication spot in the sonicator, and at optimal timing, the solution forms a cloudy appearance and the amount of solid material should be minimal as determined at this point by visual inspection.

Filtration techniques claimed include vacuum filtration for initial size selection and then extrusion of preparations for finer size selection.

### Effect of Particle Size on Target

Cholestosome component mixtures differ in novel ways depending on the ionic and physicochemical, characteristics of the macromolecular component. The size of the cargo-loaded cholestosome often affects the target in that certain cholesterol esters, when formed into cholestosomes, are better suited for delivering certain molecules and thus the impact of ester chain length.

In certain embodiments, the pharmaceutical composition is a unilamellar vesicle having a diameter of about 100 to about 750 nm, preferably about 225 to about 275 nm, and even more preferably around 250 nm. DLS measurements indicate vesicles with diameters ranging from 50 nm to more than 1000 nm. Size of vesicles is influenced by the specific macromolecular contents, the cholesteryl esters chosen for manufacture, and the temperature and sonication conditions during manufacture. The final sizing can be established by selective extrusion with an appropriate pore size as well as control of time of sonication as well as other preparation parameters.

### How the Charge and Molecular Pattern Impact the Size of the Cholestosome

Larger molecules with greater net positive charges need longer chain length cholesteryl esters for optimal encapsulation, provided the melt temperature is compatible with the stability of the molecule being encapsulated, throughout the encapsulation process. Smaller molecules with a lower net positive charge may be encapsulated with shorter chain length cholesteryl esters. Adjustment of the cholesteryl ester chain length to provide lipid vesicles based upon cholesteryl esters pursuant to the present invention is well within routine skill.

Surfaces on said cholestosomes may either be smooth, or rough, dependent on component balance and mixture characteristics. The character of the vesicle surface will depend on the esters themselves as well as the interaction of the esters with each other. The expectation is that the esters will aggregate to optimize the molecular interactions and to minimize the holes or spaces between them. These arrangements may therefore produce a surface that is rough.

Most of the graphical examples in the figures of this disclosure have rough configuration, as the esters have arranged themselves so that structural components are inter-digitated on the vesicle surface to produce an uneven structural arrangement (rough). In some cases, the esters have arranged themselves so that they are aligned to produce a surface of constant shape and size (smooth).

The nature of the final surface configuration will depend on the combinations of esters used and their relative concentration in the formulation. In summary, both the choice of esters and the choice of molecule affect the final arrangement of the lipid vesicle. While the various components affect the surface configurations, a novel surface property, the neutral surface itself that allows for uptake by enterocytes, should be the net effect of the charges of the chosen molecules in the final formulation. The surface should always be neutral

### Properties of Formed Cholestosomes and Illustrated Examples

Surprisingly, larger molecules with greater net positive charges need shorter chain length cholesteryl esters for optimal encapsulation.

Preferred cholesteryl esters for use with larger water soluble macromolecules such as proteins and peptides are those which are prepared by the esterification (or a related process to provide the corresponding cholesteryl ester) of a C₈ to C₁₄ saturated or unsaturated fatty acid, often a fatty acid selected from the group consisting of Caprylic acid, Capric acid, Laurie acid, Myristic acid.

The mixing of more than one (preferably two) cholesteryl ester to form cholestosomes may accommodate different sized active molecules with varying delivery characteristics.

FIG. 1 depicts a three-dimensional model of a cholesteryl laurate/cholesteryl myristate (1:1 molar concentration) cholestosome. cholestosomes can have a wide range of sizes, as vesicles shown here in the examples can range in size from 250 nm to 10,000 nm size (350 nm to 10,000 nm, 500 nm to 10,000 nm, 750 nm to 10,000 nm, 1,000 nm to 10,000 nm, 1,250 nm to 7,500 nm, 1,500 nm to 6,500 nm, 750 nm to 5,000 nm, 1000 nm to 5,000 nm)

Active ingredient load can be measured by measurement of hydrophilic weight to lipid weight in vesicles produced using the disclosed method. Ingredient loading may also be determined through physical measurements and calculations such as those disclosed in examples 1 and 2.

Subsequent examples with show more cholestosome preparations that pass cell membranes in the manner of FIGS. 2 to 6, but in fact when cholestosomes are absorbed into enterocytes and then passed intact into chylomicrons, the delivery inside cells is much greater. These examples illustrating greater intracellular penetration will also be shown.

Loading of Cholestosomes into Chylomicrons

Loading of cholestosomes and their molecular payload into chylomicrons by the Golgi apparatus appears to he quantitative, as evidenced by re-measurement of the apical side of the Caco-2 and subtraction of the amount remaining from the amount recovered in chylomicrons on the basolateral side. Thus, the, affinity of Caco-2 cells for cholestosomes appears to be very high. The Caco-2 cells clear all of the available cholestosomes placed on the apical side into chylomicrons on the basolateral side. As demonstrated in Example 3. Thus, the early choice of cholesteryl esters to be used to encapsulate the active molecule(s) is an essential step in the practice of the invention, and the careful application of the art disclosed herein may allow the skilled artisan to load a peptide across the enterocytes, into chylomicrons, and into body cells.

### Pharmaceutically Acceptable

The term “pharmaceutically acceptable” refers to a salt form or other derivative (such as an active metabolite or pro-drug form) of the present compounds or a carrier, additive or excipient which is not unacceptably toxic to the subject to which it is administered,

### Components in Formulations of the Invention

Formulations of the invention may include a pharmaceutically acceptable diluent, carrier, solubilizer, emulsifier, preservative and/or adjuvant. Acceptable formulation materials preferably are nontoxic to recipients at the dosages and concentrations employed. The pharmaceutical formulations may contain materials for modifying, maintaining or preserving, for example, the pH, osmolarity, viscosity, clarity, color, isotonicity, odor, sterility, stability, rate of dissolution or release, adsorption or penetration of the composition. Suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides, disaccharides, and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counter-ions (such as sodium); preservatives (such as benzalkonitun chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, polyethylene glycol (PEG), sorbitan esters, sodium lauryl sulfate, polysorbates such as polysorbate 20 and polysorbate 80, Tween was used in the present example: Tween may be used to improve the yield of emulsion prior to extrusion step: Tween can be added to the aqueous preparation prior to the addition to the lipids or to the lipid and then addition of aqueous. The smallest amount of tween possible is used, that being less than about 100 microliters in 10 ml of aqueous. Triton, trimethamine, lecithin, cholesterol, or tyloxitpal); stability enhancing agents (such as sucrose or sorbitol); tonicity enhancing agents (such as alkali metal halides, preferably sodium or potassium chloride, mannitol, or sorbitol); delivery vehicles; diluents; excipients and/or pharmaceutical adjuvants. See, for example, REMINGTON'S PHARMACEUTICAL SCIENCES, 18.sup.th Edition, (A. R. Gentian), ed.), 1990, Mack Publishing Company.

Optimal pharmaceutical formulations can be determined by one skilled in the art depending upon, for example, the intended route of administration, delivery format and desired dosage. See, for example, REMINGTON'S PHARMACEUTICAL SCIENCES, Id. Such formulations may influence the physical state, stability, rate of in vivo release and rate of in vivo clearance of the antibodies of the invention.

Primary vehicles or carriers in a pharmaceutical formulation can include, but are not limited to, water for injection, physiological saline solution or artificial cerebrospinal fluid, possibly supplemented with other materials common in compositions for parenteral administration. Neutral buffered saline or saline mixed with serum albumin are further exemplary vehicles, Pharmaceutical formulations can comprise Tris buffer of about pH 7.0-8.5, or acetate buffer of about pH 4.0-5.5, which may further include sorbitol or a suitable substitute. Pharmaceutical formulations of the invention may be prepared for storage by mixing the selected composition having the desired degree of purity with optional formulation agents (REMINGTON'S PHARMACEUTICAL SCIENCES, Id.) In the form of a lyophilized cake or an aqueous solution.

Further, the formulations may be formulated as a lyophilizate using appropriate excipients such as sucrose.

Formulation components are present in concentrations that are acceptable to the site of administration. Buffers are advantageously used to maintain the composition at physiological pH or at a slightly lower pH, typically within a pH range of from about 5 to about 8.

The pharmaceutical formulations of the invention can be delivered parenterally. When paremeral administration is contemplated, the therapeutic formulations for use in this invention may be in the form of a pyrogen-free, parenterally acceptable aqueous solution. Preparation involves the formulation of the desired immune-micelle, which may provide controlled or sustained release of the product which may then he delivered via a depot injection. Formulation with hyaluronic acid has the effect of promoting sustained duration in the circulation.

Formulations according to the present invention may be formulated for inhalation. In these embodiments, a stealth Cholestosome-molecule formulation is formulated as a dry powder for inhalation, or inhalation solutions may also he formulated with a propellant for aerosol delivery, such as by nebulization. Pulmonary administration is further described in PCT Application No. PCT/US94/001875, which describes the pulmonary delivery of chemically modified proteins and is hereby incorporated by reference.

Formulations may be formulated for topical application on the skin. In these embodiments, a stealth Cholestosome-molecule formulation is formulated as an ointment or cream, and applied to the surface of the skin.

Formulations of the invention can be delivered through the digestive tract, such as orally and this represents a preferred route of administration. The preparation of such pharmaceutically acceptable compositions is disclosed herein and within the skill of the art. Formulations disclosed herein that are administered in this fashion may be formulated with or without those carriers customarily used in the compounding of solid dosage forms such as tablets and capsules. A capsule may he designed to release the active portion of the formulation at the point in the gastrointestinal tract when bioavailability is maximized and pre-systemic degradation is minimized. Enteric coatings which are stable to acid but degradable within a pH of the duodenum (about 5.0 to about 6.0 or slightly higher) may be preferred. These are well known in the art. Additional agents can be included to facilitate absorption. Diluents, flavorings, low melting point waxes, vegetable oils, lubricants, suspending agents, tablet disintegrating agents, and binders may also be employed.

A formulation may involve an effective quantity of a cholestosome, most preferentially a cholestosome formulation and a molecule in a pharmaceutical composition as disclosed herein in a mixture with non-toxic excipients that are suitable for the manufacture of tablets. By dissolving the tablets in sterile water, or another appropriate vehicle, solutions may be prepared in unit-dose form. Suitable excipients include, but are not limited to, inert diluents, such as calcium carbonate, sodium carbonate or bicarbonate, lactose, or calcium phosphate; or binding agents, such as starch, gelatin, or acacia; or lubricating agents such as magnesium stearate, stearic acid, or talc.

The pharmaceutical composition to be used for in vivo administration typically is sterile. In certain embodiments, this may be accomplished by filtration through sterile filtration membranes. In certain embodiments, where the composition is lyophilized, sterilization using this method may be conducted either prior to or following lyophilization and reconstitution. In certain embodiments, the composition for parenteral administration may be stored in lyophilized form or in a solution. In certain embodiments, parenteral compositions generally are placed into a container having a sterile access port, for example, an intravenous solution bag or vial having a stopper pierce-able by a hypodermic injection needle.

Once the formulation of the invention has been formulated, it may be stored in sterile vials as a solution, suspension, gel, emulsion, solid, or as a dehydrated or lyophilized powder. Such formulations may be stored either in a ready-to-use form or in a form (e.g., lyophilized) that is reconstituted prior to administration.

Administration routes for formulations of the invention include orally, through injection by intravenous, intraperitoneal, intracerebral (intra-parenchymal), intra-cerebroventricular, intramuscular, intra-ocular, intra-arterial, intra-portal, or intra-lesional routes; by sustained release systems or by implantation devices. The pharmaceutical formulations may be administered by bolus injection or continuously by infusion, or by implantation device. The pharmaceutical formulations also can be administered locally via implantation of a membrane, sponge or another appropriate material onto which the desired molecule has been absorbed or encapsulated. Where an implantation device is used, the device may be implanted or topically applied into any suitable tissue or organ, and delivery of the desired molecule may be via diffusion, timed-release bolus, or continuous administration.

Protection of Molecular Payloads from Acid and/or Enzymatic Degradation in the GI Tract.

Cholesteryl ester vesicles themselves survive intact at pH values ranging from 2-14, in contrast to liposomes which are rapidly degraded by these same conditions and are relatively unstable compared to compositions according to the present invention. Cholestosomes prepared with labile payloads may be coated with an outer enterically targeted layer in cases where their payload constituents must be protected from degradation in the gastrointestinal tract so that the cargo-loaded cholestosomes reach the duodenum (G.I. sites of entemcytes which produce chylomicrons incorporating the cholestosomes).

Payloads such as insulin and other proteins/polypeptides are acid labile, necessitating an additional step of an enteric coating protective of insulin in cholestosomes, to be applied prior to use in animal or in vivo systems where there is potential for acid or enzymatic degradation. Under usual situations in the practice of the art, when the contents of a cholestosome are acid labile peptides and proteins, and when these products are cholestosome encapsulated in preparation for oral ingestion, there should be a final product administered with an emetic coating to protect the contents of the cholestosomes from the acid in the stomach. In most cases after release of the cholestosomes in the duodenum there is the possibility of enzymatic degradation or bile salt mediated saponification in the duodenum, so there is a need to perform stability studies of the individual cholestosomes in contact with bile salts, pancreatic lipases and pancreatic esterases. Therefore, unless or until the protein or peptide is definitively proven to be free of acid degradation, the dosage form will be a small capsule filled with cholestosome construct, then coated with enteric coating to release contents at pH 5.5 to 6.0. A suitable coating for this purpose would be Eudragit (19, 20) or another enteric polymer which is stable to acid but having similar degradation characteristics to the Eudragit polymers and while cholestosomes themselves are stable in low pH, there remains a need to employ enteric coatings known in the art to protect the contents of cholestosomes from acid degradation.

Enteric Coatings

As used herein, “enteric coatings” are substantially insoluble at a pH of less than a range of between about 5.0 to 7.0 to about 7.6 (preferably about 5.0 to about 6.0 or slightly more within this range), and can be comprised of a variety of materials, including but not limited to one or more compositions selected from the group consisting of poly(dl-lactide-co-glycolide chitosan (Chi) stabilized with PVA (poly-vinylic alcohol), a lipid, an alginate, carboxymethylethylcellulose (CMEC), cellulose acetate trimellitiate (CAT), hydroxypropylmethyl cellulose phthalate (HPMCP), hydroxypropylmethyl cellulose, ethyl cellulose, color con, food glaze and mixtures of hydroxypropylmethyl cellulose and ethyl cellulose, polyvinyl acetate phthalate (PVAP), cellulose acetate phthalate (CAP), shellac, copolymers of methacrylic acid and ethyl acrylate, and copolymers of methacrylic acid and ethyl acrylate to which a monomer of methylacrylate has been added during polymerization.

Once macromolecules are incorporated into cholestosomes, the product would be placed in a capsule. This capsule may or may not be emetic coated, depending on the site of intended release of the cholestosome composition. By way of example, an enteric coating may be applied to said capsule to protect against acid in the stomach and release the cholestosome composition at pH 5.5 or thereabouts. By way of a second example as applied herein for cancer immunotherapeutic use of cholestosomes, the enteric coating applied would release the cholestosome in the ileum at pH values between 7.3 and 7.6.

Enteric coatings can be applied to said capsules by conventional coating techniques, such as pan coating or fluid bed coating, using solutions of polymers in water or suitable organic solvents or by using aqueous polymer dispersions. As an alternative embodiment, the release controlling enteric coating can be applied to capsules within capsules, each containing separate composition and designed to release sequentially. One preferred embodiment of said release would be outer capsule target release at duodenum at pH of 5.5, and an inner capsule release at the ileum at pH 7.3 to 7.6. By way of example, suitable materials for the release controlling layer include EUDRAGIT® (copolymers of acrylic and methacrylic acid esters), EUDRAGIT®RS (copolymers of acrylic and methacrylic acid esters), cellulose derivatives such as ethylcellulose aqueous dispersions (AQUACOAT®, SURELEASE®), hydroxyethyl cellulose, hydroxypropyl cellulose, hydroxypropyl methylcellulose, polyvinylpyrrolidone, polyvinylpyrrolidone/vinyl acetate copolymer, OPADRY®, and the like.

These and other aspects of the invention are described further in the following non-limiting examples.

### NON-LIMITING ExampleS OF THE PRESENT APPLICATION

### Example 1

### Step by Step: Preparation and Testing of Cholesteryl Ester Vesicles

### Cholesteryl Ester Vesicle Preparation

This example shows the Steps in the preparation of a cholesteryl ester vesicle for eventual oral use of a molecule, an insulin as disclosed herein. One skilled in the art will appreciate that these conditions will be suitable for use with a GLP-1 agonist peptide with only minor experimentation. The resulting vesicle for encapsulation of an oral drug molecule, oral protein, oral peptide, oral gene or nucleic acid construct of genetic material (the term “molecule” used to define one or all of these hereinafter in this example) may be prepared as follows:
1. Obtain purified cholesteryl esters and composition elements for encapsulation;
2. Obtain molecule targeted for encapsulation and pre-test for purity and stability at 37° C.-55° C.;
3. Optimize components of cholesteryl esters in the vesicle mixture using a computer model of interactions between esters and molecule to achieve maximum vesicle loading of said molecule;
4. Optionally, prepare vesicle encapsulated molecule and include Fluorescein Isothiocyanate (FITC) label for purposes of conducting biological studies including, microscopy, said FITC label not a component of product intended for human testing or therapeutic use;
   Studies of Cholesteryl Esters and Molecules Considered for Use In Formation of vesicles.

This example applies in general to cholesteryl ester preparation for encapsulation of peptides, proteins and genes, most of which must be loaded into vesicles at temperatures below 55 degrees centigrade. As this temperature may not be exceeded because the encapsulated molecule is not stable, the selection of cholesteryl esters for use in the practice of the invention is critically dependent on use of a liquid form of the cholesteryl ester in the formulation. Steps in selection of candidate cholesteryl esters are as follows:

Define the melting point of each ester. By way of example, cholesteryl myristate has a melt transition temperature of 65 degrees centigrade, above which temperature the solid component melts.

The formulation objective was to use cholesteryl esters at temperatures below the melt temperature, (Consistent with liposome preparations), and considering that many proteins and peptides begin to denature at temperatures about 40 degrees centigrade or above.

Further temperature testing was carried out on the chosen esters myristate and laurate. After the organic solvent was completely removed from the lipids in the roto-vap, a DSC was conducted, which showed two melting temperatures, one approximately 60 degrees centigrade and a second melt at a higher temperature, typically 80 degrees centigrade. This step is not reflected in the individual component melts, rather this reflects a test of stability of the organization of the, lipids in a 1:1 molar ratio, once the organic solvent has been removed.

On the basis of these finding and considering the stability of the proteins and peptides being formulated, including insulin by way of example, the operating temperature of encapsulation procedures was kept,between 40 and 55 degrees centigrade. Insulin was shown to be stable across this range of temperature. By way of example, optimal insulin preparation was at 37-55 degrees centigrade.

### Selection of Cholesteryl Ester Pairs Between C6 and C22 and Compositions for Encapsulation of Molecules

Selection of specific cholesteryl esters for the proper formation of encapsulating vesicles involves a novel approach and a computerized molecular model. Properties of the cholesteryl esters and the interaction between the target molecule for encapsulation and the inner hollow core of vesicle thrilled from the esters around the molecule can be used'to define favorable cholestosome-molecule properties such as loading, either on a volume to volume basis or a weight to weight basis.

The relevant volume in the vesicles available for encapsulation is a function of the chain length of the esters. The diameter of the entire vesicle is modified as a function of the experimental conditions, energy supplied in sonication for example. But the relevant volume is determined by the inner radius. The inner radius is determined by the length of the sterol nuclei of one component of the pair packed with the other to the sterol nuclei of that other member pair. The inner radius is the difference between the outer radius of the vesicle and the length of the ester pair chosen and packed. The inner radius decreases in length as the chain length increases. An increase in chain length increases the hydrophobic character of the ester wall. The outside sterol nuclei and the inner exposed sterol nuclei are farther away from each other as the chain length increases.

In picking an ester pair for optimal encapsulation, several factors need to be considered including the charge/diploe and size of the molecule to be encapsulated. This is then compared to the electrostatic distribution of the esters in the wall as a function of chain length. Ester pairs are then chosen based on the electrostatic compatibility with the molecule in an aqueous environment

### Formulation of Cholestosomes:

Cholestosomes can be formed using cholesteryl esters that range from short chains to long chains. We have specific examples herein with the range between C6 and C22.
b) Cholestosomes can be formed using cholesteryl ester pairs that differ by more than 2CH2 units.
c) Solubilization of the ester pairs in the lipid component can be used with organic solvents whose choice is a function of the solubility of the ester pair chosen including chloroform and ether.
d) These ester pairs can form vesicles of different sizes and can deliver drugs/molecules into cells.
e) Mole fractions of the esters can vary.
f) The temperature at which the organic solvent is removed can range from 40 to 65, again as a function of the mole ratio of the esters and a function of the solvent, the former most important in most applications.
g) Solubilization of the esters can use different amounts of organic solvent.
h) Formulation process:
   1) Add the desired mass of each ester to a round bottom (RB) flask (of various sizes) and add appropriate amount of organic solvent.
   2) Attach RB flask to rotovap and place in water bath of desired temperature. Rotate for 20 minutes.
   3) Turn on vacuum to remove organic solvent
   4) When solvent is removed add aqueous component which has been in a water bath of desired temperature.
   5) Sonicate for 20 minutes
   6) Filter and recover lipid and filtrate (prep)
   7) Remove unencapsulated drug with methods such as dialysis, filtration as well as others.

FIG. 1B, Table 3 summarizes the results of testing of mixtures of short, intermediate and long chain fatty acid cholesteryl esters, and the figures to follow illustrate the impact of these cholesteryl ester vesicles on MCF-7 cells

Mixtures of Two Short Chains Differing by Two CH2 units (C6/C8)

In FIG. 2, Cholestosomes made by combining two short chain Cholesteryl esters that differ by two CH2 units, Cholesteryl Caprylate (C6) and Cholesteryl Caprate (C8), mixed in a 1:1 molar ratio. The cholestosomes resulting from this combination were an average size of 350 nm when prepared in ether and when FITC was incorporated into these cholestosomes, the green fluorescence showed that they entered cells.

### Mixture of Two Long Chains Differing by 4 CH2 (C18/C22)

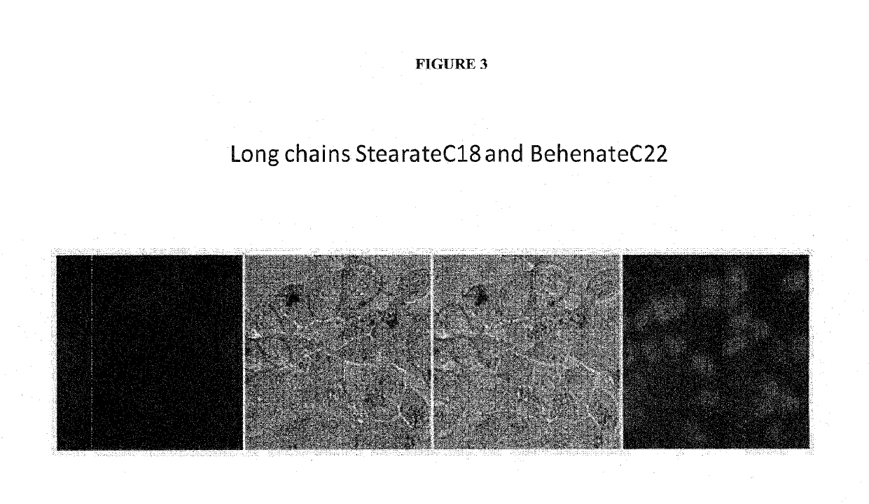

In FIG. 3, Cholestosomes made by combining two long chain Cholesteryl esters that differ by four CH2 units, Cholesteryl Stearate (C18) and cholesteryl Behenate (C22), mixed in a 1:1 molar ratio. The cholestosomes resulting from this combination ranged in size from 392 nm if prepared at 65 C in chloroform to 3899 nm if prepared at 55 C in ether when FITC was incorporated into these cholestosomes, the green fluorescence showed that they entered cells.

### Mixture Differing By 10 CH2 Units (C12/C22)

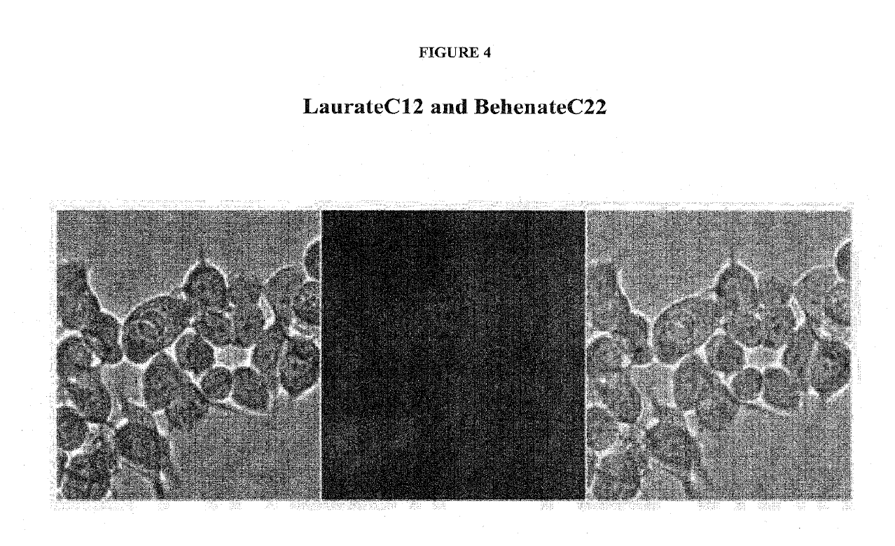

In FIG. 4. Cholestosomes made by combining two Cholesteryl esters that differ by ten CH2 units, Cholesteryl Laurate (C12) and Cholesteryl Behenate (C22), mixed in a 1:4 molar ratio. The cholestosomes resulting from this combination had an average size of 1500 nm when prepared in chloroform at 65 C. When FITC was incorporated into these cholestosomes, the green fluorescence showed that they entered cells.

Mixture Differing By 8012 units (C14/C22)

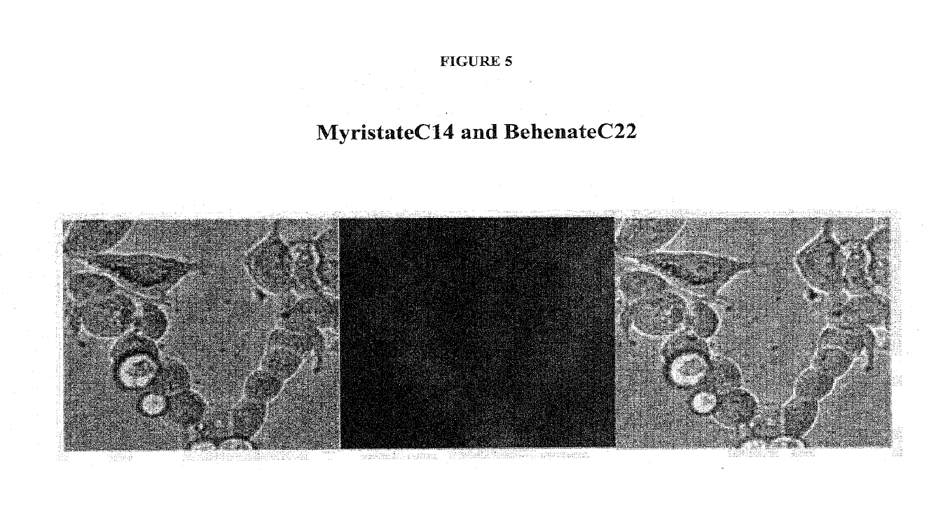

In FIG. 5, Cholestosomes made by combining two Cholesteryl esters that differ by Eight CH2 units, Cholesteryl Myristate (C14) and Cholesteryl Behenate (C22), mixed in a 4:1 molar ratio. The cholestosomes resulting from this combination had an average size of 690 nm when prepared in chloroform at 65 C. When FITC was incorporated into these cholestosomes, the green fluorescence showed that they entered cells.

### Mixture Differing By Two CH2 Units (C14/C16)

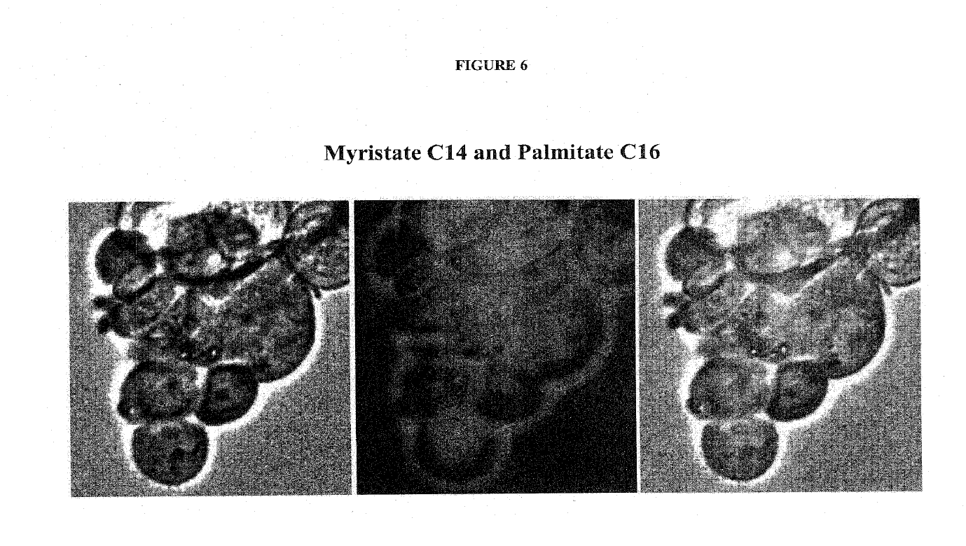

In FIG. 6, Cholestosomes made by combining two medium chain Cholesteryl esters that differ by two CH2 units, Cholesteryl myristate (C14) and Cholesteryl Palmitate (C16), mixed in a 1:1 molar ratio. The cholestosomes resulting from this combination were an average size of 1890 nm when prepared in chloroform and when FITC was incorporated into these cholestosomes, the green fluorescence showed that they entered cells.

### Preparation of 12 C:14 C Cholestosome Control:

5 mL of PBS buffer is added to a 15 mL centrifuge tube and warmed to 55° C. in a water bath, 80.0 mg of Cholesteryl Myristate and 75.0 mg of Cholesteryl Laurate are added to a 100 mL round-bottom flask (RBF) and solubilized with 5 mL of diethyl ether (a 1:1 molar ratio which has been determined by DSC and phase diagram construction). The RBF containing the 155.0 mg of cholesteryl esters and diethyl ether is then held in a Buchi Rotavapor R-3 system that is pre-heated to 55° C. The esters are exposed to low vacuum at speed setting 4 for 20 minutes to remove the solvent. The RBF is then removed after the vacuum seal is released. Preheated 5 ml of PBS is added to the esters in the RBF and it is sonicated for 20 minutes in an S Series Ultrasonics Sonicor or Elmasonic P pre-heated to 55° C. The Cholestosome solution is filtered through a 40 um Falcon cell strainer into 15 mL centrifuge tube and stored at 4° C.

### Assumptions and Procedure for Loading Calculation of Cholestosomes:

The loading of molecule in the vesicle is determined after analysis of several parameters including mass of lipid in final formulation as determined by HPLC, average size of vesicles as determined by microscopy and DLLS and the starting concentration of the aqueous component of the preparation.

In the typical practice of the invention, the ester volume in the bilayer model will be a small percentage of the total volume.

The total diameter is variable nm depending on experimental conditions;

The width of the ester bilayer is 2.5 nm, so the total volume minus the inner volume should equal the volume occupied by the lipids.

So radius outer layer −2.5 nm is the radius of the inner layer.

Steps in the calculation of encapsulated volume:

a) Measure diameter of particle using DLS or microscopy.

b) Calculate outer radius by dividing diameter by 2.

c) That is Rₒᵤₜ

d) Rᵢₙ=Rₒᵤₜ−2.5 nm

e) calculate volume occupied by esters (assume a spherical particle) 4/3*pi*[(Rₒᵤₜ)³−(Rᵢₙ)³]=volume occupied by esters.

f) The volume of an individual ester calculated using SYBYL is 640.45 Â3, converting to cubic nanometers is 0.640 nm̂3.

g) Divide volume occupied by esters from part by volume of individual esters to calculate the total number of esters in a Cholestosome.

h) Calculate mass of one ester: first, find, the average mass of a myristate and laurate molecule:

for Cholesteryl myristate MW=597.0092; for cholesteryl laurate MW=568.9560; so, average=582.98 g/mole. Then, find the mass of one ester=582.98 g/mole div by 6.022 E+23 esters/mole 9.680880106276984 e-22 g/ester. Multiply this by the number of ester/cholestosome in part g) to find the mass of one Cholestosome.

i) Most of the cholestosome is inner core containing the active pharmaceutical agent. Determine the mass of lipid in a prep based on HPLC method. Take the total mass of lipid and divide by mass of one Cholestosome from part h) to determine the number of cholestosomes.

j) Determine volume of inner core, which is the volume occupied by encapsulated molecule.

V_{inner sphere}=4/3*pi*(Rᵢₙ)³

k) Convert volume of cholestosome in nm³ to ml

1 ml=1 cm³

10⁶ cm³=1 m³

10²⁷ nm³=1 m³

l) Multiply volume from k by number of cholestosomes calculated from i).

m) That is the encapsulated volume per volume of preparation

n) Multiply encapsulated volume from m by starting concentration of aqueous molecule in formulation. That gives the encapsulated mass of material per volume of prep of per mass of lipid.

### Example 2

### Cholesteryl Ester Composition for Insulin

### Steps and Method for Thin Film Preparation of Insulin in Cholestosomes.

This example shows the Steps in the preparation of an insulin encapsulated in a cholesteryl ester vesicle for eventual oral use. By way of specific example, Human Recombinant Insulin made in bacteria (Prospec labs, Israel) cholestosomes were prepared in the manner of the present invention, as described in Example 1, with cholesteryl ester selection from the esters disclosed as preferred in Example 1, One skilled in the art will appreciate that these conditions will be suitable for use with a GLP-1 agonist peptide with only minor experimentation and adjustment for different molecular properties. The resulting vesicle encapsulating an oral drug molecule, oral protein, oral peptide, oral gene or nucleic acid construct of genetic material (the term “molecule” used to define one or all of these hereinafter in this example) may be prepared as disclosed herein,

Multiple batches of cholestosomes containing insulin were prepared using the optimized blend of two cholesteryl esters, cholesteryl myristate and cholesteryl laurate in a 1:1 mole ratio. The choice of cholesteryl esters for composition is made from the disclosed compounds of Example 1, optimized for insulin by means of the loading calculations,

Although this is not meant to be limiting, after reasonable experimentation there are other suitable pairs of cholesteryl esters for formulation with insulin or similar molecules, and they may be permitted in the practice of the invention.

In the specific preparation of an optimal cholestosome formulation containing insulin, any cholesterol ester may be chosen as a component of the cholestosome and be within the spirit of the invention so long as the final Zeta Potential of the cholestosome product retains its neutral or slightly negative surface charge. The two esters chosen for insulin using the principles disclosed in Example 1 were cholesteryl myristate and laurate, which differ in ester chain length by two CH2 units, and when combined as disclosed provide a large internal hydrophilic center to the cholestosome vesicle prepared in this manner. This pair is in the middle of the chain length of the fatty acids. Other chain lengths may work as well, given the less hydrophobic nature of the shorter chain length vesicles and the remarkable property of cholestosome formation from cholesteryl esters of greater than two CH2 units.

Optimizing the amounts of specific cholesteryl esters is fully within the scope of the present invention for purposes of producing an optimal loading and release profile of the insulin containing cholestosome for in vivo use.

Initial starting conditions are based on a 1:1 molar ratio of laurate/myristate, although the final ratio in the formulation of the various insulin molecules is not limited to that. Each insulin molecule will need to be examined in terms of its own structure and the molecular interactions with the putative cholesteryl esters as a means of final selection of cholesteryl esters for optimal loading. In the event the optimal final formulation requires a more hydrophobic area, then a longer chain fatty acid ester is used, as the entire proportion of hydrophobic space will change based on the length of the alkyl chain. If we need more centralized hydrophilic structures for certain insulin molecules, the intention is to use one of the oxysterols such as 25 hydroxy, 7-keto or other hydroxy cholesterols made into an ester with fatty acids.

The encapsulation molecule is insulin, to include but not limited to regular insulin, NPN insulin, insulin glargine, insulin degludec or any formulation of insulin prepare and shown to be bioactive in testing for insulin effects, Steps in preparation of the cholestosome formulation included the following:

Prepare a water bath to appropriate temperature (37-55)C; Place aqueous insulin prep (8 mg/ml) in PBS into water bath to equilibrate temperature; Weigh out equimolar amounts of cholesteryl laurate and cbolesteryl myristate (75 mg each) and place in round bottom flask; Add organic solvent (diethyl-ether) to dissolve esters: swirl by hand to dissolve; Place round bottom flask on rotovap and spin for five minutes; Place flask attached to rotovap in water bath; turn on vacuum and spin for 10 minutes; Turn off rotovap and vacuum and add aqueous to round bottom flask; Optionally add Tween; Spin on rotovap (no vacuum) for twenty minutes in water bath; Sonicate for 10 to 30 minutes until cloudy preparation is formed and only minimum solid lipid remains in the round bottom flask; Remove from sonication and filter using vacuum filtration; Save the cloudy filtrate; Optionally Extrude filtrate; Store preparation in refrigerator until use.

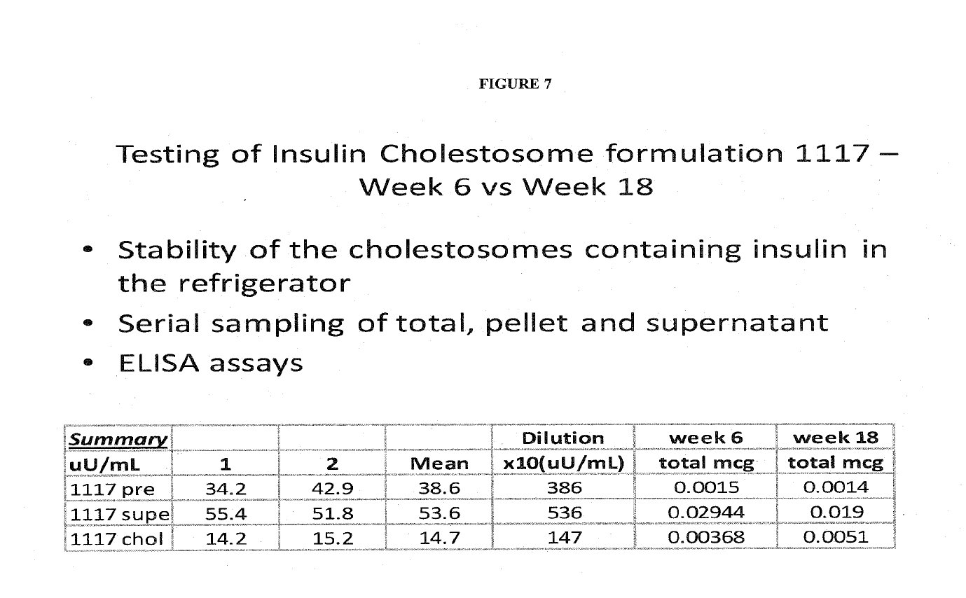

In the refrigerator, formulation 1117 was stable for at least 18 weeks, as shown in FIG. 7.

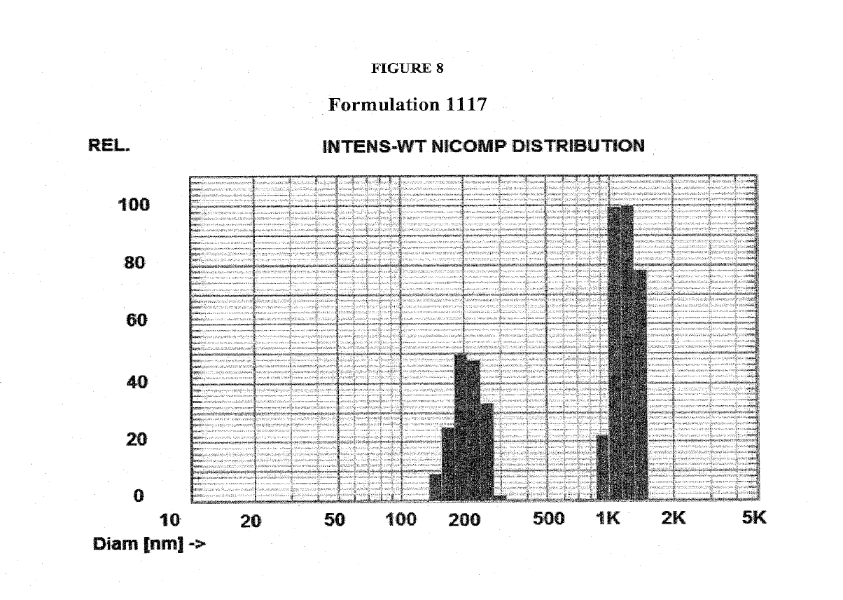

DLS analysis of Particle size was carried out using a NICOMP 380 Submicron Particle sizer (Brookhaven). A sample of the output of said NICOMP on formulation 1117 is shown as FIG. 8, where there were two particle size distributions, one averaging 208 nm and the second averaging 1191 nm.

### Alternative Preparation of Insulin-Cholestosomes

Insulin-Cholestosomes were also prepared without formation of a lipid layer in a round bottom flask. This preparation was made by the inventors using a 15 ml reaction vessel containing 8.0 mg/ml insulin solution. Five ml of the insulin solution held in a water bath sonicator at 37 degrees centigrade while an ether solution of the lipids was slowly infused, and continuous vacuum was applied to remove the ether. After 10-20 minutes of sonication, the now very cloudy reaction mixture was removed from vacuum and processed to define particle sizes and to remove aggregates. Tween 20 was added to the mixture to decrease the aggregates and to facilitate separation of unencapsulated insulin by centrifugation. This preparation yielded high amounts of encapsulated insulin with few aggregates, and over 300 U of insulin was encapsulated in a one hour period without the need for a thin film step.

### pH and Insulin Solubility In the Preparation of Cholesteryl Ester Vesicles Using Thin Film Method

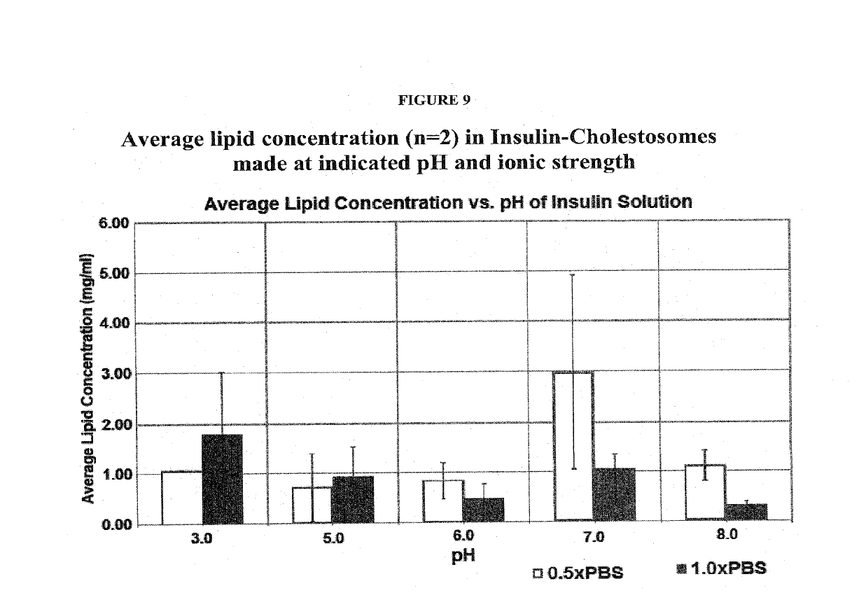

Many preparations of cholestosome insulin have been made. Upon inspection of these data, pH appeared to affect loading and potentially solubility of the insulin used in the preparations. Accordingly, the solubility limits of Insulin were empirically tested at pH 3.5, 6, 7 and 8. Data on the lipid content of each formulation are plotted by pH of the insulin solution are plotted at two different ionic strengths of PBS buffer in FIG. 9.

The maximum concentration at each tested pH was applied to the following: Stock solutions for each pH and ionic strength combination are prepared. The empirically determined maximum concentration of Human Recombinant Insulin (SAFC Biosciences and. ProSpec) was added to the stock solution. These stock solutions were then readjusted to the desired pH using either 1M HCl or 1M NaOH as needed 5.0 ml of stock solution was heated to 55° C. and used as the aqueous component in preparations.

### Insulin-Cholestosomes Were Prepared Using Thin Film Lipid Layers.

The preheated 5.0 mL of 1× PBS in the 15.0 mL Crystalgen centrifuge tube was added to the ester solution. Sonication of the aqueous mixture was carried in an S Series Ultrasonics bath sonicator (Sonicor, W. Babylon, N.Y.). Sonication is done for 20 minutes, with 90 degree rotations of the round bottom flask every 5 minutes.

After the 20 minutes, a pre-weighed Falcon cell strainer is used to drain the cholestosome solution back into the original 15.0 mL Crystalgen Centrifuge Tube, which is then stored in a standard refrigerator. The pre-weighed round-bottom flask is placed in a VWR drying unit set at 37 degrees Celsius. The final weights of the 15.0 mL Crystalgen. Centrifuge Tube and the Falcon cell strainer are recorded 24 hours after use in order to calculate an approximate % yield for the amount of lipid. Characterization and analysis of the lipid cholestosomes is done through utilization of Dynamic Laser Light Scattering, (DLLS; NanoBrook, Brookhaven Instruments, Inc., Holtzville, N.Y.) and High-Pressure Liquid Chromatography, HPLC (Prominence 2020 LC-MS, Shimadzu, Tokyo, JP).

Size of Cholestosomes:

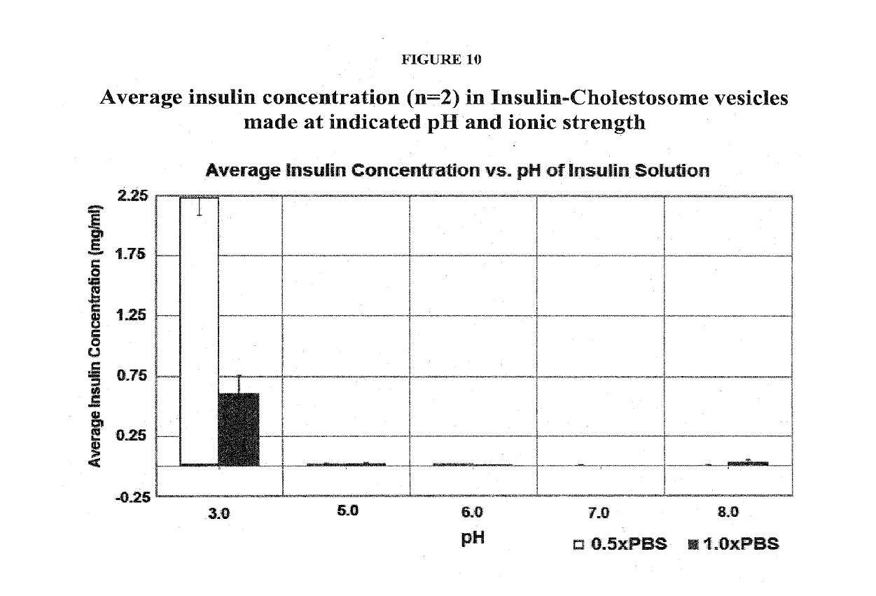

At acidic pHs (3,5& 6) size increases as ionic strength increases. Conversely, the size of Cholestosomes at basic pH (8) increases as ionic strength decreases. Size of pH (7) Cholestosomes is unaffected by ionic strength, as shown in FIGS. 9 and 10.

Encapsulation Efficiency (E.E.):

E.E. of formulations at pH (6-8) is maximized at higher ionic strengths. E.E. of pH (3&5) Cholestosomes is maximized using the ionic strength of1× PBS, as shown in FIG. 11.

pH (8) Cholestosomes:

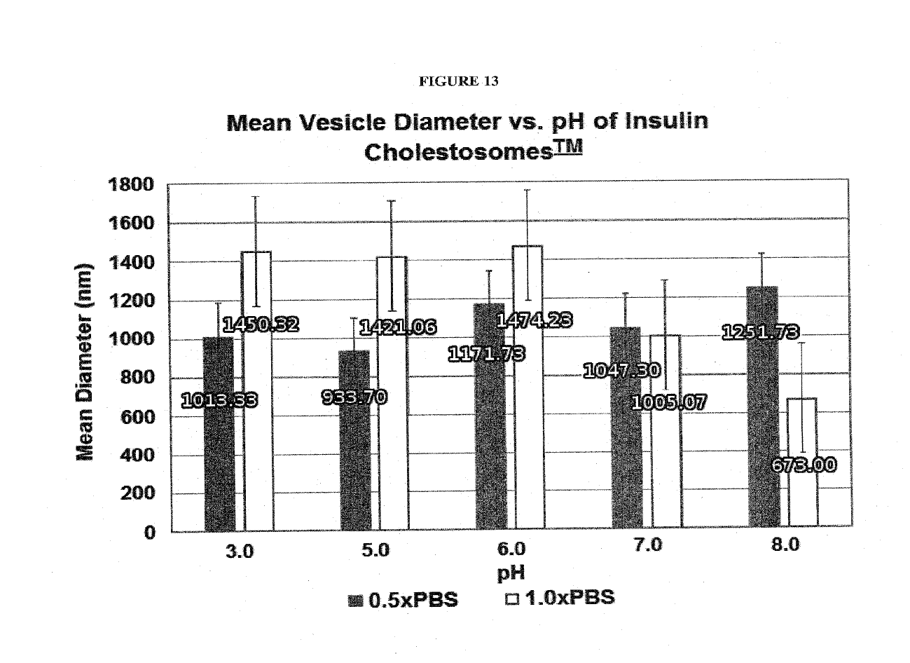

Insulin solubility is high at pH 8.0, and the concentration encapsulated within Cholestosomes increases at basic pH (8) as ionic strength increases. Unfortunately, HPLC analysis has shown that the pH (8) environment breaks down the insulin A/B Dimer making these formulations unfavorable, as shown in FIG. 13.

pH (3-4) Cholestosomes:

Insulin concentration encapsulated is maximized at high and low ionic strengths. Conversely, lipid concentration is maximized at the central ionic strength, as shown in FIGS. 11 to 13.

Conclusions:

Because encapsulated insulin is maximal across the range of ionic strengths tested, and there was relatively consistent lipid incorporation as well as size parameters across the range of pH, due to the instability of insulin at pH 8.0, the production of formulations at pH of 3-4 is a preferred embodiment.

-pH 3 insulin formulation used in this study (1117) was orally bioavailable in mice, as will be demonstrated in Example 4.

### Processing of Reaction Mixture to Remove Aggregates

After sonication of the reaction mixture for 10-20 minutes, the reaction mixture was treated with a surfactant (Optionally Tween 20 in the preferred embodiment for insulin), mixed, and centrifuged. The surfactant added was used to adjust the reaction mixture so that it remained homogenously disbursed after centrifugation and removal of the first supernatant, the insulin which is unencapsulated. When it was observed to be homogenously disbursed, and a microscopic examination showed even dispersion of particles, with few aggregates, the mixture was ready for filter separation of unencapsulated insulin from the insulin-cholestosome containing reaction mixture.

Removal of Unencapsulated Insulin from Insulin-Cholestosome Mixture

When surfactant is used, Insulin cholestosomes may be separated from supernatant by centrifugation. At this point the Insulin cholestosomes are free of aggregates and generally can be evenly disbursed in PBS and stored in the refrigerator until used. However, the free insulin in the mixture may be further removed by filtration or dialysis.

Filtration used an 0.45 u hydrophilic PVDF Sterile Filter to remove the excess insulin which was not encapsulated. This mixture was washed on the filter and then separated from the filter and stored for use as cleaned product insulin-cholestosomes. The filter separation allowed for the additional use of the insulin stock solution. Some Preparations were dialyzed using a 300 kDa MWCO dialysis tubing against 1× PBS at neutral pH to remove unencapsulated insulin. Preparations made in pH 3 1× PBS were also dialyzed, while pH 3 preps in 0.5 × and 2.0× PBS were filtered using a 0.45 u hydrophilic PVDF Sterile Syringe: Filter. Filtered material was recovered by reversing flow.

### Microscopy for Aggregates and Particle Sizes

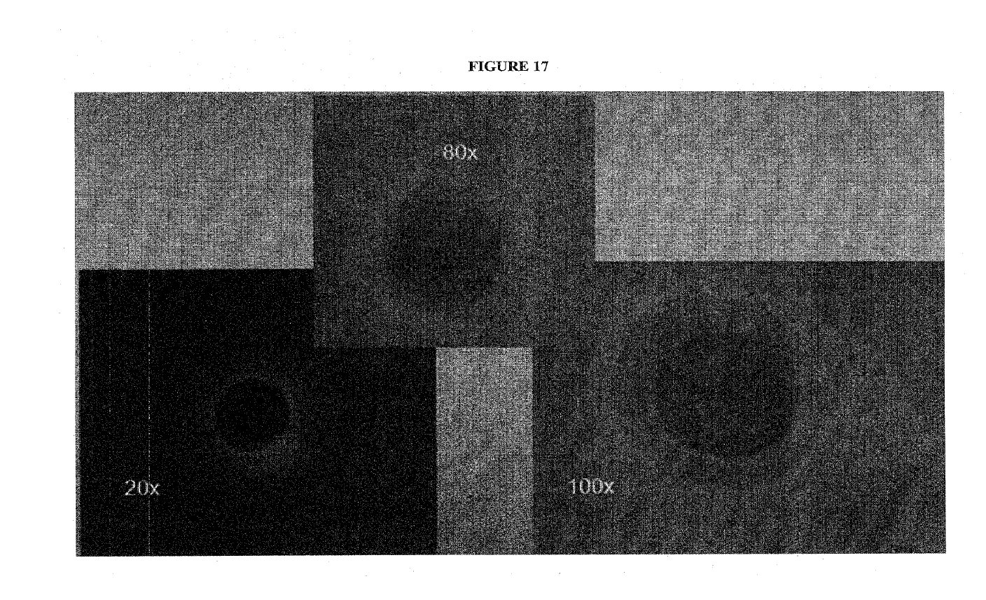

Initial sizing of the reaction mixture was performed by dark field microscopy, which effectively visualizes particles between 500 nm and 5000 nm in size. There are images provided of insulin formulations where aggregation is visible under light microscopy, such as FIG. 14. Further processing of the sample to use surfactants in increasing amounts does not change the size of the particles but does decrease the aggregation, as shown in FIG. 14. A surfactant dispersed insulin-cholestosome particle distribution of an average size approximately 1.7 microns, ranging from 1.6 to 1.8 is shown as FIG. 15. There is a higher power image of the particles shown as FIG. 16, where it can be appreciated that the particles are comprised of an outer radius and an inner radius. The content of the inner core and the outer membrane can be calculated and used to define particle loading, as will be detailed below in the loading calculations. Further analysis of these particles subjected them to Scanning electron microscopy, as shown in FIG. 17. Here it is much easier to visualize the nature of cholesteryl ester particles with a large hydrophilic core at the center. Consistent with the calculations below, the core of this particle is over 50% of the contents of the particle, in the example presented for insulin below, the core represents 69% of the contents of the entire vesicle.

### Calculations Defining Loading of Insulin-Cholestosomes: 2000 nm Vesicles

These calculations refer to cholestosome insulin prepared as in Example 2. Assume preparation aqueous solution of insulin has an 8 mg/ml starting insulin concentration. Assume HPLC measures a 1 mg/ml lipid in the prep (total mass of cholesteryl laurate and cholesteryl myristate).
A Measured diameter of vesicle is 2000nm.
Outer Radius=Rₒᵤₜ=1000 nm
Inner Radius=Rᵢₙ=1000 nm−2.5 nm=997.5 nm
Rₒᵤₜ³=1,000,000,000 nm³
Rᵢₙ³ 992518734.4 nm³
Volume of esters=4/3*pi*(1,000,000,000−992518734.4)=31 21565 nm³
Number of esters=31321565/0.640=48939945.8
Mass of esters in one Cholestosome=(48939945.8)*(9.6800×10⁻²²)=4.737×10⁻¹⁴ grams*1000 mg/1 gram)=4.747×10⁻¹¹ mg cholesteryl esters in one Cholestosome
Assume 1 mg/ml lipid from HPLC measurement of lipid content.
1 mg/(4.747×10⁻¹¹)=2.1×10¹⁰, the number of cholestosomes in one ml.

Calculation of Inner Volume
4/3*pi*(Rᵢₙ)³=4/3*pi(992518734.4 nm³)=455345101 nm³
Convert to ml (4155345101 nm³)*(1 m³/10²⁷ nm³)*(10⁶ cm³/1 m³)*(1 ml/1 cm³)=4.155×10⁻¹² ml per Cholestosome
(2.1×10¹⁰)*(4.155×10⁻¹²)=0.087 ml encapsulated volume per ml of prep 8 mg/ml*(0.087)=0.698 mg insulin/ml of preparation 1 mg lipid per ml prep; so mass to mass in this instance is 0.698 mg insulin/1 mg lipid per ml of preparation for an encapsulation of 69.8%.

In the practical application of the technology, a loading range of 25% to about 96%, most likely 25% to 80% by w:w of insulin to total particle size, is within the scope of the present invention.

### Final Yield: A Concentration of Approximately 20 Units of Insulin Per ml of Prepartnon, Consisting of Vesicles of 1500 nm to 3000 nm In Diameter.

Structure of a Cholestosome with Encapsulated Insulin By Example

Shown in FIG. 1 in modeling and by transmission EM in FIG. 16, is a loaded cholestosome structural model with encapsulated insulin (formulation 1117) as an example. It is assumed that these ideal lipid particles are aggregated into clumps of lipid, with raw production sizes of clumps of about 1000-5000 nm. While the inventors have discovered that particle sizes between 1000 nm and 3000 mn work well for loading large molecules into cells and are orally bioavailable in mammals (insulin by way of example), extrusion of these tame particles down to uniformly sized 250 nm particles is also a preferred embodiment. This can be effected using a standard high-pressure extrusion device, well known in the art.

### Example 3

### In Vitro Cell Testing Methods for Cholestosome Encapsulated Peptides, Proteins, and Genetic Materials

### Overview of Cell Testing Methods

The uptake by enterocytes and incorporation into chylomicrons is an essential component of high oral bioavailability shown by the vesicles invented for this purpose. This example details the, steps of preparation and administration of Insulin Cholestosomes (from Example 2) to an in-vitro means of testing the cellular uptake and chylomicrons incorporation properties conveyed by choice of cholesteryl esters. The testing system begins with an enterocyte model system, for purposes here the Caco2 cell monolayer, whereby the cholesteryl ester vesicles with their encapsulated molecules are used to pass the Caco-2 cell membranes, and then incorporated into chylomicrons, which can he measured in basolateral fluids collected from Caco-2 cells using MCF-7 cells.

Steps that occur after cholesteryl ester vesicle encapsulation of said molecule (an insulin by example) and after cholesteryl ester vesicle encapsulation of said FITC labeled molecule (an insulin by example) are as follows:
1. Test FITC labeled molecule in Caco2 cell monolayer and collect chylomicrons encapsidated FITC-cholestosome-molecules on basolateral fluid after 24 hrs, now defined as incorporated into cholestosome loaded chylomicrons;
2. Expose test cells (MCF-7 cells by example) to chylomicrons containing FITC-cholestosome-molecules and determine uptake of FITC-molecule by these test cells. While MCF-7 cells are often chosen because of their ease of use and relevance to cancer, workers will realize that testing many different cell lines for uptake in the case where cellular targeting is a subject of scientific investigation, as intracellular uptake of many bioactive molecules is novel and unanticipated from prior art in the field of drug delivery;

3. Define, using microscopy, whether intracellular FITC-molecule is contained in endosomes or it is free in cytoplasm; Early time points (2 hr, 4 hr) will show even distribution of FITC throughout the cell, indicating no endosomal step. Typical later time points for imaging of endosomes is approximately 24 hr after the initial exposure, and endosomes can be visualized at 48 hr and even 72 hr, although by this late time most cells have removed endosome FITC labeled contents by exocytosis.
4. Define, using Western Blot expression of GLUT-transporters, whether the intracellular action of molecule is expressed as cell surface mediated uptake of additional substances or molecules controlled by actions of intracellular molecule:

### Caco-2 Cell Culture

Caco-2 intestinal epithelial cells were gown in Dulbecco's Modified Eagle's Media (DMEM) supplemented with 20% fetal bovine serum (heat inactivated), 1 nM sodium pyruvate, 100 U/ml penicillin, 100 μg/ml streptomycin, 292 μg/ml L-Glutamine and 1× MEM non-essential amino acids (Invitrogen, #11140). Cells were cultured in 75 cm² flasks and incubated in a 37° C. and 5% CO2 environment until 80-90% confluent. The Biocoat HTS Caco-2 assay system (Becton Dickson, #0354802) was used for transport studies. Cells were seeded at a density of 3×10⁵ cells/well and differentiated into enterocyte-like cells using, the manufacturer's protocol. This system used a special fibrillar collagen insert and a serum free differentiation medium enhanced with butyric acid, hormones, growth factors and metabolites to establish a differentiated monolayer in 3 days instead of the 21 days that is normally required for this process. This involved incubation of the cells with a DMEM based media containing 10% FBS for 48 hours at 37° C., 5% CO₂ after seeding to establish a growth phase. The media was then replaced by the defined differentiation media and incubated for another 48 hours. At this point the Caco-2 cells are ready for further studies.

### Chylomicrons Production by Caco-2 Cells

The differentiated Caco-2 cells were used for the production of Chylomicrons using the method of Luchoomun and Hussain-1999 (21). This method uses supplementation with oleic acid (OA) and taurocholate (TC) at a 1.6:1.0 mM ratio to achieve chylomicrons production. The OA:TC solution was made up in the DMEM based cell growth media used in the BIOCOAT differentiation protocol by adding OA to a 10 mM TC solution and incubated at 37° C. until clear. This was then 0.2 u filtered and used immediately for the transport studies. Treatments plus OA:TC were added in a 450 ul volume to the apical side of the well and incubated for 24 hours for transport studies and chylomicrons production. PBS plus lingind glucose was added in a 1ml volume to the basolateral side. Assessment of the integrity of the tight junctions of the Caco-2 monolayer was performed using 100 mM Lucifer Yellow (Life Technologies). The cellular monolayer was lysed with 100 ul/well of lysis buffer and incubated 10 mitt at 37° C. All samples were stored at 4° C. until measurement. Fluorescence levels of apical, basolateral and cellular FITC -insulin were determined using a LS45 Luminescence Spectrophotometer (Perkin Elmer).

Chylomicrons levels were assessed using a Human ApoB ELISA Kit (Cell Biolabs, #STA-368) [16]. The Human ApoB-100 Standard was aliquoted and stored at −20 degrees Celsius while all other components were stored at 4 degrees Celsius. The. 1× Wash Buffer was prepared by diluting the 10× Wash Buffer Concentrate to 1× with deionized water and stirring until homogeneous. The Anti-ApoB Antibody and Secondary Antibody was prepared by diluting the Anti-ApoB antibody 1:1000 and Secondary Antibody 1:1000 with Assay Diluent immediately before use. A dilution series of human ApoB-100 standards was prepared in the concentration range of 0 to 50 ng/mL in Assay Diluent. The first standard tube contained 2 uL of 50 uL/mL Human ApoB-100 Standard and 1998 uL of Assay Diluent. The remaining 7 standard tubes were prepared with 500 uL of the previous standard with an addition 500 uL of Assay Diluent added. The basolateral serum was harvested and centrifuged for 10 minutes at 1000 g at 4 degrees Celsius. The serum required 20,000 fold dilution with PBS containing 0.1% BSA immediately before running the ELISA. 100 uL of ApoB unknown sampler standard was added to the Anti-ApoB Antibody Coated Plate with each ApoB unknown sample, standard and blank being duplicated twice. The plate was then incubated at 37 degrees Celsius for 2 hours. The microwell strips were washed 3 times with 250 uL 1× Wash Buffer per well. The wells were then emptied and microwell strips were the tapped on paper towels to remove access Wash Buffer. 100 uL of diluted anti-ApoB antibody was then added to each well and incubated at room temperature for 1 hour on an orbital shaker. The wells were then washed 3 times as previously done. 100 uL of diluted secondary antibody was then added to each well and again incubated at room temperature of 1 hour on an orbital shaker. The wells were again washed 3 times. The Substrate solution was warmed to room temperature and 100 uL of Substrate solution was added to each well, including the blanks. Again, the plate was incubated a room temperature on an orbital shaker until the reaction was complete. The reaction was stopped by adding 100 uL of Stop Solution into each well, including the blank wells. The absorbance of each well was read using at spectrophotometer with 450 nm as the primary wavelength.

### Caco-2 Studies in Transwell, and Formation of Chylomicrons

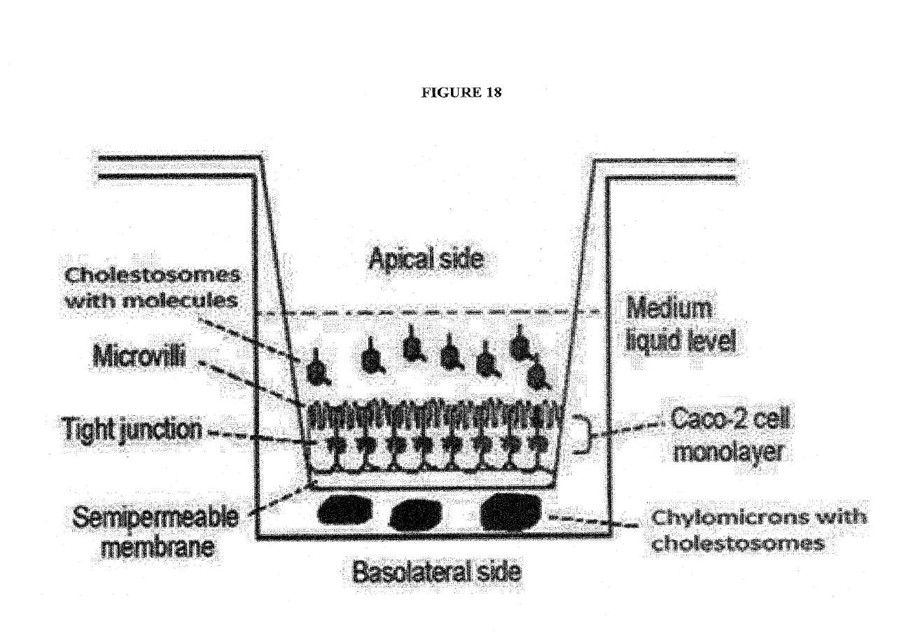

FIG. 18 demonstrates the basic setup of a Transwell plate, which is designed to show passage of a substance across a monolayer of Caco-2 cells. In the case of cholestosome insulin and other cholesteryl ester preparations, we are testing uptake by Caco-2 cells followed by insertion of the intact cholesteryl ester vesicle into the chylomicrons of the Caco-2 cells. This is a novel use of the Caco-2 cell monolayer, but within the scope of the art since it is known that Caco-2 cells make chylomicrons under the conditions stated above.

In the practice of the art, we employ Coming Transwell Permeable Supports in a 12 well format with a pore size of 0.4 um. We begin each Transwell experiment after Caco-2 cells are 80-90% confluent in a 75cm² flask. The cells are trypsinized as usual and counted using a hemocytometer. The cell concentration is adjusted to 2×10⁵ cells/ML with culture media. The wells of the Transwell plate are seeded with 0.5 mL of the cell dilution. Media in a volume of 1.5ml is added to the basolateral side. The cells are incubated as above and the media is changed every other day for 19-20 days. At this time the Caco-2 cells are differentiated and ready for treatment. All media from the upper and lower chambers of the Transwell plate is removed and both chambers are washed 3 times with PBS containing 1 mg/mL glucose (PBSG). PBSG is added to the upper and lower chamber of the plate and incubated for 1 hr. All PBSG is removed from both chambers and 1.5 mL of phosphate buffered saline with added glucose (PBSG) is added to the lower chamber.

The upper chamber, the apical side of the Caco-2 cell monolayer, receives 0.5 mL of the appropriate treatment (PBSG alone, FITC cholestosomes in PHSG or FITC-insulin cholestosomes in PBSG.). All wells have a final concentration of 1.0 mg/mL glucose. The plate is then incubated for 2 hours. All solution is removed and viewed on the Zeiss confocal LSM 510 microscope.

Imaging of the basolateral fluid following RTC insulin cholestosomes applied to the apical side for 2 hr, shows a predominance of overall larger chylomicrons containing FITC-insulin in the basobteral fluid samples. It is important to note that this fluid was imaged after collection of the basolateral fluid and does not reflect microscopy across the entire preparation. Hence, these FITC insulin containing chylomicrons were clearly formed by the Caco-2 cells.

### FITC-Insulin vs FITC-Cholestosome Insulin Across Caco-2 Cells

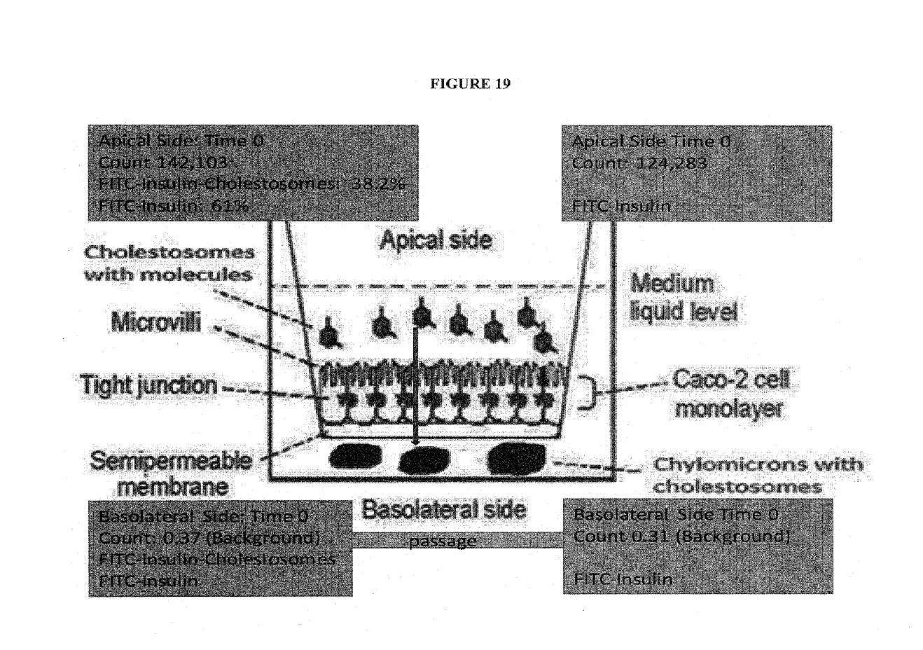

The FITC signal of FITC-insulin and FITC-Cholestosome Insulin were compared after 24 hours of being placed on the apical side of the Caco-2 cell apparatus, in order to track the process of crossing the apical membrane and being ejected as chylomicrons on the basolateral surface. As shown in FIG. 19, nearly 100% of the unencapsulated FITC-insulin placed on the apical side of the transwell remained there after the 24 hour time period. This is concluded because it can be seen in FIG. 19 that only about 1% of FITC signal for F1TC- insulin is found in the Caco-2 cells and less than 1% of the FITC signal for FITC-insulin was recorded in the basolateral layer. Again, this finding confirms that free-insulin is cannot efficiently pass through Caco-2 cells.

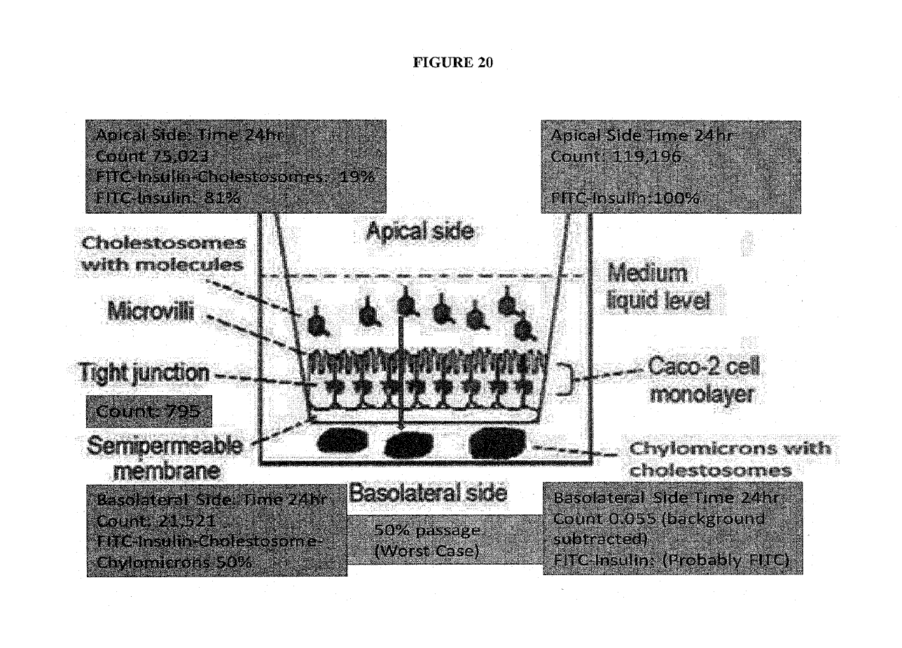

FIG. 20 shows the counts if 50% of FITC-insulin in cholestosomes remains on the apical side, as it would occur if the FITC-insulin-cholestosome mixture still contains free insulin. We term this the worst case scenario for passage across the Caco-2 monolayer into basolateral fluid.

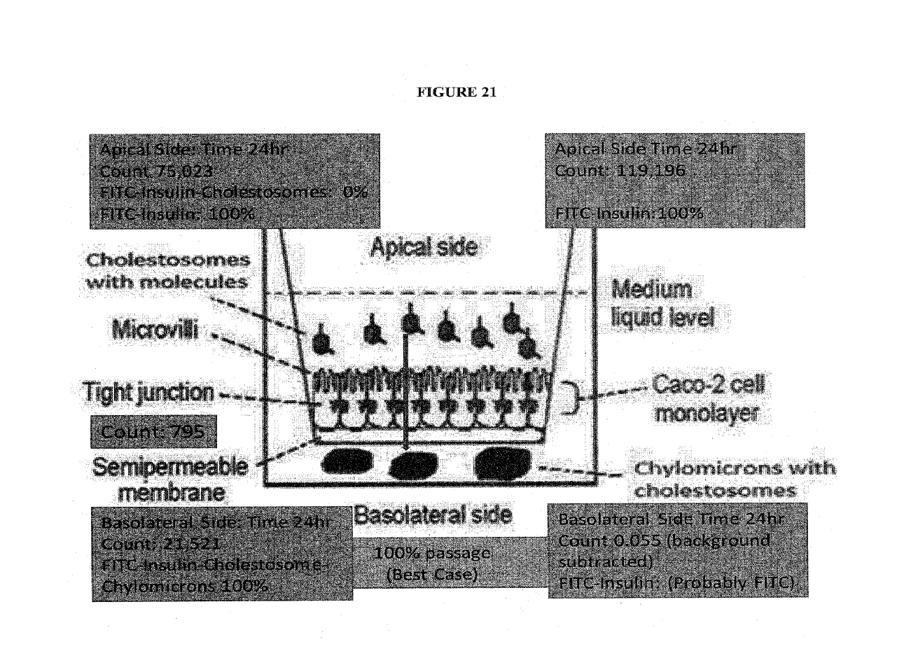

FIG. 21 shows the best case, where all of the FITC-Insulin-Cholestosomes on the apical side is encapsulated in cholestosomes, and all of it passes across into the basolateral fluid. In the practice of the invention and testing of the products, the problem of aggregates creates some free FITC insulin in most of our FITC-insulin cholestosome preparations. Thus the actual case is probably between 50% passage of the Caco-2 on the worst case and 100% passage in the best case. It is a lot like bioavailability to be shown later in both mice and rats. The presence of free insulin complicates the measurement. The final analysis was from the fluid of the basolateral layer shows a nearly untraceable FITC signal from FITC-insulin further supporting the evidence that free insulin is unable to bypass CaCo-2 cells, while FITC-Cholestosome Insulin has the ability to be transported through the cells

Overall, at least and about 50% of FITC-insulin cholestosomes placed on the apical side, was on the basolateral side after 24 hours. We consider this a conservative estimate, as some of the FITC-insulin-cholestosome preparation, about 81% was actually free FITC-insulin and not encapsulated. Clearly, while traveling through the Caco-2 cells, FITC-Cholestosome Insulin becomes incorporated into chylomicrons.

This data confirm that free insulin does not have the ability to be transported through the Caco-2 cells, which is consistent with its very low bioavailability in mammalian species.

When insulin was encapsulated in cholestosomes, however, it was able to be transported across the Caco-2 layer. This is significant because it shows that insulin encapsulated in cholesteryl ester vesicles has the potential ability to be transported out of the digestive system and into the bloodstream where the insulin could be transported to peripheral cells.

### Culture of MCF-7 Cells

The insulin cholestosome preparations, both with and without FITC, needed a cell line for regular testing. We chose MCF-7 cells for this purpose, as they are easy to grow and have many features of standard cell lines.

MCF-7 epithelial breast cancer cells were obtained from ATCC. Cells were grown in DMEM supplemented with 10% heat inactivated fetal bovine serum, 1 nM sodium pyruvate, 100 U penicillin, 100 U streptomycin, 292 ug/ml glutamine and 10 ug/ml gentamycin. Cell monolayers were maintained in 75 cm² flasks in a humidified 37° C., 5% CO₂ environment. Media was renewed every 48-72 hours to provide optimal growth conditions. Cells were then analyzed using an EVOS FL Cell imaging System (Life Technologies).

### Cholestosome FITC Insulin Loaded on MCF-7 Cells

The initial MCF-7 cell study was done by loading FITC insulin Cholestosomes into the cell culture media of the MCF-7 cells, and incubating to determine the amount of insulin that could be transported inside the cell, as well as to track the time course of its uptake.

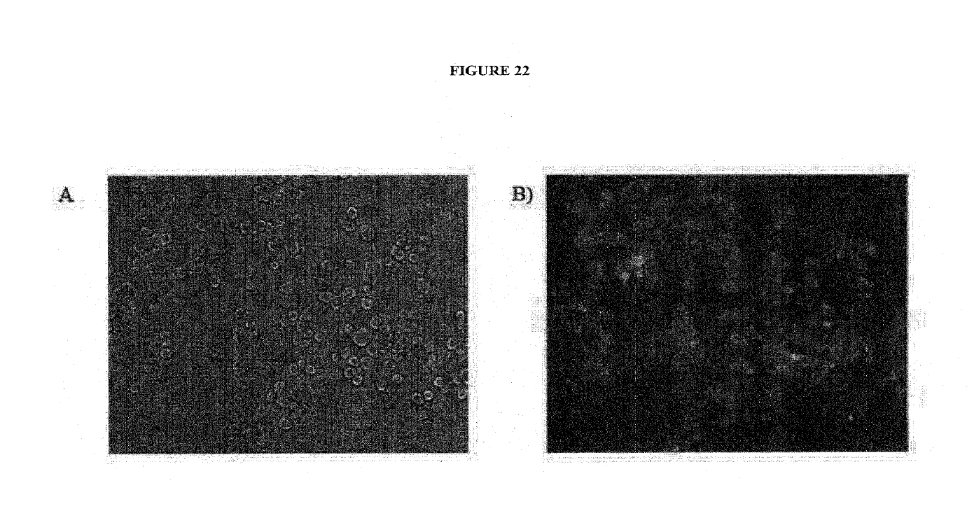

FIG. 22 shows the phase contrast and the fluorescence images of the MCF-7 cells 24 hours after being loaded with FITC-Cholestosome Insulin. Frame A shows the darkfield image, and Frame B shows the fluorescence. FITC insulin cholestosomes are prominently diffuse throughout the cell, but it is also possible to see many small spherical exocytosis vesicles throughout Frame B. At least some of these contain FITC insulin still intact, because there is detection of FITC insulin in mouse plasma for prolonged periods after oral administration of FITC insulin cholestosomes.

The fluorescence of the MCF-7 cells, uniformly green glow, indicates that a sufficient number of the FITC-Cholestosome Insulin vesicles were transported into the cell, and favors a membrane transporter for the cholesteryl vesicle. This was a significant finding because it shows that Cholestosome Insulin itself efficiently delivered insulin to cells over 24 hours, and that uptake of FITC insulin in cholestosomes ( )UM; even without a chylomicrons step. Furthermore, early time points in the time course do not show any endocytosis vesicles. Thus there is evidence that cell uptake of FITC insulin cholestosomes quickly lead to release of FITC insulin free in the cytoplasm without any evidence of endocytosis. In fact, the distribution of FITC label remains uniform until about 6 hrs, when there is the beginning of cell exocytosis.

Time course studies showed that the peak of the fluorescence in MCF-7 cells was found at around 18 hours. When delivered orally however, Cholestosome insulin would first need to bypass the intestinal wall before reaching the peripheral cells, which are mimicked by the MCF-7 cells, in order to deliver the insulin. Therefore, the next step in the experiment was to place Cholestosome insulin on Caco-2 cells, which mimic the enterocyte process of absorption of cholesteryl ester vesicles and placement into chylomicrons. Men the basolateral fluid with its load of chylomicrons could be tested for the expected entry into MCF-7 cell when they receive their cholesteryl esters from chylomicrons (a mimetic of the effect of oral use of cholestosomes).

### FITC Cholestosome Chylomicrons Loading MCF-7 Cells

Cholestosomes containing encapsulated FITC-insulin were prepared as disclosed herein, using FITC labeled regular insulin purchased commercially. Caco-2 cells were used to ensure that Cholestosomes transfer intact insulin (i.e. insulin remains within the Cholestosome) across the enterocytes and enters chylomicrons, following which chylomicrons were detected on the basolateral side of the Caco-2 membrane. ELISA was used to demonstrate that acid protected insulin does not pass the apical Caco-2 barrier (<5%), and that all of the insulin on the basolateral side is within chylomicrons. FITC-insulin was used on the apical side to verify that insulin alone does not pass the enterocyte barrier but that FITC insulin in cholestosomes passes the Caco2 enterocyte barrier. From these experiments, absorption efficiency was determined as the difference between basolateral side, and apical side content of insulin Further experiments compared the effect of altered pH and bile salts on the cholestosome encapsulated insulin. In addition, chylomicrons stability when containing insulin loaded into cholestosomes was quantified and the conditions necessary for release of insulin from the loaded cholestosomes in vivo were studied.

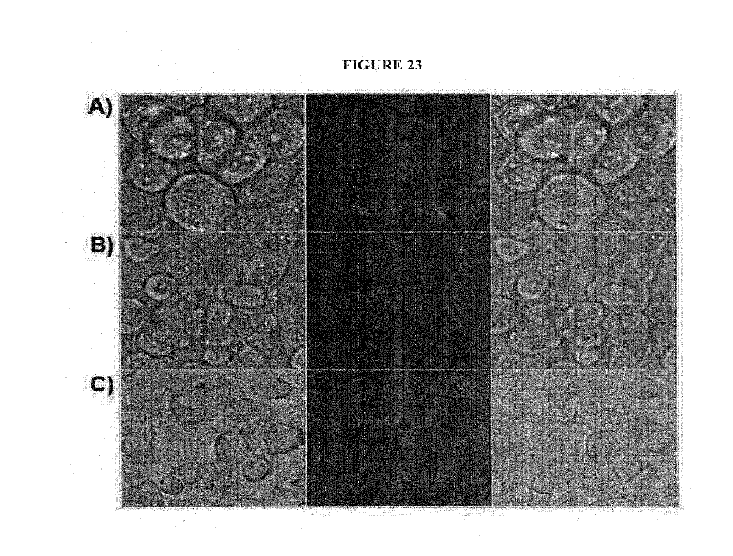

In FIG. 23, the FITC insulin preparations were placed in the media adjacent to MCF-7 cells in order to demonstrate uptake into cells. This image was taken at 2 hr after adding the construct to the media. FIG. 22 compares the ability of unencapsulated FITC-insulin, row A, FITC-Cholestosome Insulin, row B, and FITC -Cholestosome Insulin in chylomicrons, row C, to deliver FITC-insulin into MCF-7 cells. All rows have a darkfield image first, the fluorescent image in the middle and the last image is an overlay of the fluorescence over the darkfield.

In this experiment FITC insulin cholestosome chylomicrons loading of MCF-7 cells was nearly 1000× greater (row C) as compared to loading from FITC-insulin alone (row A). There was modest but detectable loading in Row B from FITC insulin cholestosomes given without the chylomicrons step. Throughout FIG. 23, it can be appreciated that at 2 hrs there are no visible endosomes as well as uniform distribution of FITC insulin throughout the cells.

In all cases, the in-vivo processing of FITC insulin cholestosomes by Caco-2 cells into chylomicrons, produces a robust improvement in the amount of insulin inside cells. This is greater than loading from FITC insulin cholestosomes by themselves (row B),

Not only are the cell membranes dramatically more concentrating FITC insulin in row C of the image, but the cytoplasm of these cells is loaded with FITC insulin as well. This is after only 2 hr exposure, confirming that chylomicrons not only load massively more, they load more quickly than cholestosomes on their own.

This particular formulation was then administered to mice, in order to confirm the Caco-2 model prediction of high oral bioavailability. That aspect of the invention is disclosed in Example 4.

### Example 4

### In Vivo—Murine Studies of Cholestosome-Insulin

The experiments disclosed in Example 4 were designed to confirm the predicted high oral bioavailability from the use of Insulin in cholestosomes.

### ELISA Analysis of Insulin

ELISA analysis was performed as per manufacturer's instructions, using the Human Insulin ELISA Kit (#EZHI-14K and #EZHI-14 BK, Dattustadt, Germany). The 10× concentrated HRP Wash. Buffer was diluted by mixing the entire content with. 450 mL deionized water. The strips were removed from the Microtiter Assay Plate and the wells were filled with 300 uL of diluted HRP Wash Buffer. The plate was incubated at room temperature for 5 minutes. The wells were washed with Wash Buffer and 20 uL of Assay Buffer was added to the NSB (non-Specific Binding) wells and each of the sample wells. 20 uL of Matrix Solution was added to the NSB, Standard and Control wells. 20 uL of Human Insulin Standards were added in duplicate. 20 uL of QC1 and 20 uL of QC2 were added to appropriate wells. 20 uL of the unknown samples were added in duplicate to the remaining wells. 20 uL of Detection Antibody was added to all wells, which were then covered with plate sealer and incubated at room temperature for 1 hour on an orbital plate shaker. The plate sealer and decant solution were removed from the plate and the wells were washed 3 times with diluted 300 uL HRP Wash Buffer per well per wash. 100 uL of enzyme solution was added to each well and the plate was again covered with the sealer and incubated at room temperature for 30 minutes on an orbital shaker. The sealer and decant solutions were removed and the wells were washed 5 times in the same way. 100 uL of Substrate Solution was added to each well, which were again sealed and put on the orbital shaker for 5-20 minutes. 100 uL of Stop Solution was added to the wells after a blue color appeared indicating that the reaction was complete. The absorbance of each well was read using at spectrophotometer with 450 nm as the wavelength. The measurements were recorded as micro Unit per ml.

### Oral Delivery of Cholestosome Insulin to Mice

This example presents data collected after mice were given oral or IV cholestosome insulin.

The Insulin was administered as free (unencapsulated) human recombinant insulin, or as cholestosome encapsulated human recombinant insulin. Of the mice given insulin-cholestosomes for measurement of relative bioavailability (oral to IV expressed as percent AUC), half were dosed orally while the other half were dosed IV using a 31gauge needle into the penile vein.

The dosing and sacrificing of the mice used followed a strict humane procedure. A dose of 1.0 U/kg of human insulin recombinant was administered in 0.05-0.1 ml saline to ND4 Swiss Webster mice (Harlan Laboratory, Indianapolis, Ind.). The mice were fasted for 3 hours prior to administration and allowed access to food after one hour. Water was provided at all times to the mice. The, mice received anesthesia by inhaling isoflurane prior to the penile vein injection. 3% mg/Kg body weight was inhaled by the mice for induction of exsanguination and 2% mg/Kg was inhaled for maintenance. After the Insulin administration, the mice were monitored every 30-60 minutes for the first 6 hours, then every 12 hours, with behavior and urination strictly recorded. Two mice per time, point were then sacrificed by inhalation in a CO₂ chamber.

The time points in which the mice were sacrificed were 1.0 (pre) and post at 30 min, 1 h, 3 h, 6 h, 12 h, 24 h. Blood samples were collected by aortic artery puncture, exsanguination, in the sacrificed mice.

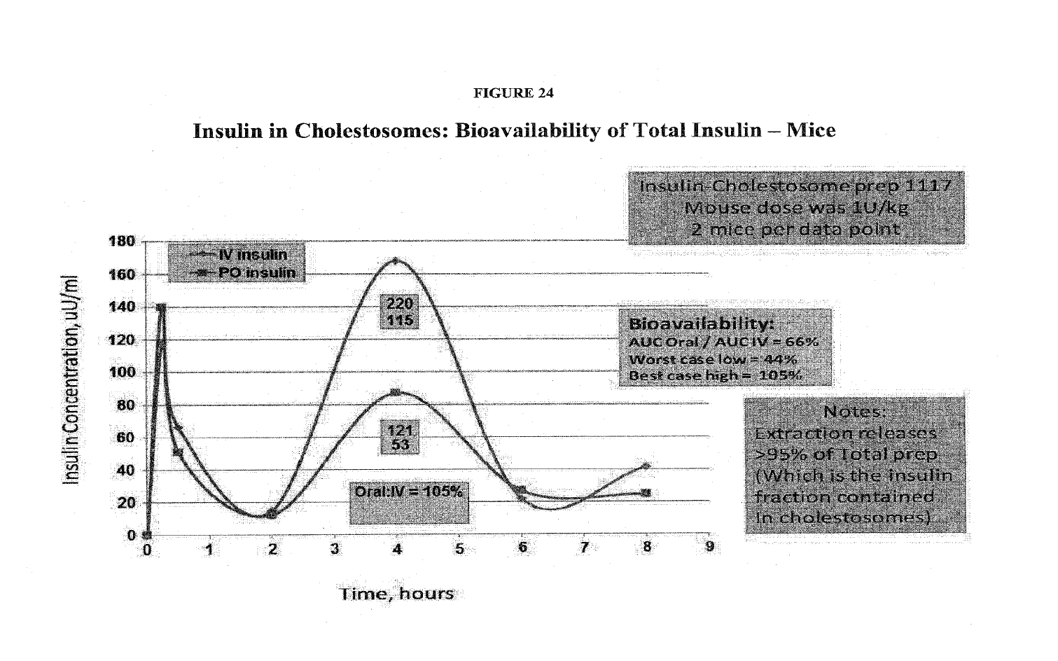

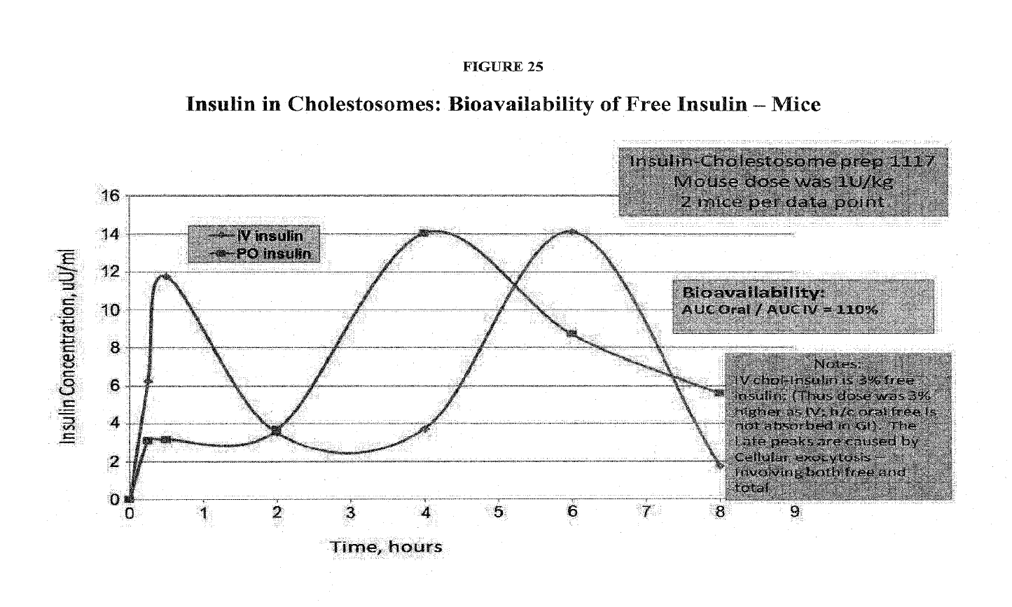

FIGS. 24 and 25 show the results of mice given 1.0 U/kg of Cholestosome Insulin both orally and IV for purposes of comparing bioavailability. FIG. 24 plots Cholestosome Insulin (Total, assuming extraction was complete) while FIG. 25 plots insulin that is free of cholestosomes after release inside the cell and exocytosis. Using relative AUC as a measure of Oral to IV bioavailability, the oral free insulin was 110% bioavadable, and even more when corrected for the 3% free insulin in the formulation.

A large exocytosis associated peak of insulin was noted between 4 and 6 hours after oral AND IV dosing, indicating that some of the intact cholestosomes that enter cells are also transported intact back to the outside without opening them and releasing their contents inside the cell.

Considering the plots of Cholestosome encapsulated insulin as FIG. 25, the total cholestosome encapsulated AUC associated bioavailability averaged 66%, again associated with a very large exocytosis peak of still encapsulated insulin cholestosomes at 4 hr after both oral and IV dosing. It was expected that cholestosomes would all be releasing intracellular free insulin, while the data argue that cells simply eject at least a portion of the cholestosomes that enter them after chylomicrons docking.

Given the initial understanding of cholestosome transport, more studies are necessary to optimize the delivery. Also, there was significant variability from mouse to mouse. The information recorded in FIGS. 24 and 25 is nonetheless important, however, because it validates our model and our in-vitro data, as well as indicates that Cholestosome Insulin has the ability to be orally delivered into a mammal with a very high bioavailability, and in fact considerably higher than the usual 5-25% which is the best previously available value.

As a further note on the mechanisms of oral and IV cholestosome entry and exit from the cells, Insulin concentration (uU/mL) was determined by collecting plasma from the mice and performing an Insulin ELISA using the Millipore kits. It is important to note than any insulin measured by the ApoB EL/SA was insulin delivered as either free insulin or Cholestosome insulin, because the ApoB ELISA kit is specific to human recombinant insulin. This is important, as the mice used in the study were not diabetic meaning they also had their own naturally produced insulin, although the Millipore kits for human insulin assay announce that they do not cross over to mouse insulin. If it could not be determined whether the insulin readings were from the mice or from Cholestosome Insulin, the results would have been inconclusive. Because human recombinant Insulin has ApoB proteins attached to it, while normal insulin produced in the body does not, the ApoB ELISA kit could determine the insulin that was specifically delivered from Cholestosome Insulin in chylomicrons, which also contain ApoB as part of their cellular docking mechanisms.

### Tissue to Plasma Ratios Mouse Studies

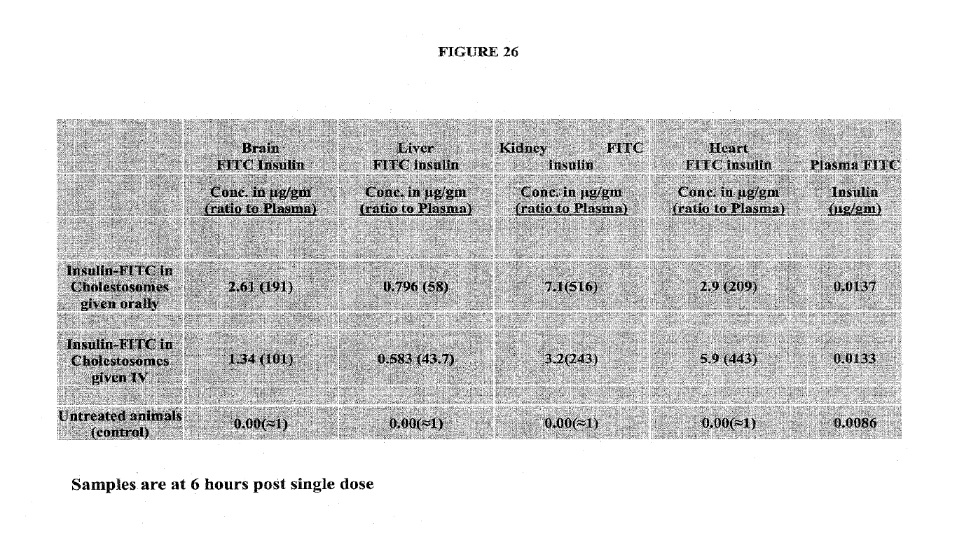

As shown in FIG. 26, cholestosome encapsulated FITC insulin shows high absorption into tissues after oral use, and in fact the concentrations in target tissues such as Brain, Liver, Kidney and Heart are higher after oral dosing than after the same dose given IV. This is expected because oral use places the FITC-insulin into chylomicrons and chylomicrons presumably load cells better than cholestosomes that are injected.

The assays here were detecting FITC according to a FITC calibration standard applied to tissue. Samples of tissues were taken 6 hr after dosing, at a time when plasma concentrations of insulin are low according to the data in FIG. 24. Accordingly, the assays of FITC reflect intracellular FITC since blood and presumably Interstitial fluid contamination of the tissues in question are absent. Tissue concentrations were particularly high in Brain, where ratios of plasma to tissue FITC were above 100 to 1. Kidney and heart were even greater as ratios of Tissue to plasma exceeded 200 to 1. Liver was lower, but still above 50 to 1. It a summary, at a time when FITC and recombitant human insulin was nearly undetectable in plasma, the ratio of Tissue to Plasma remained very high. As insulin is rapidly metabolized by cells, these ratios reflect retention of intracellular FITC, and are entirely consistent with the long retention of FITC in our cell experiments, where the concentration may still be high 24hr after exposure to FITC cholestosome insulin in the media.

### Example 5

### In vivo—Rat Studies of Cholestosome-Insulin

### Oral Bioavailability Studies

These experiments were conducted to evaluate the comparative plasma and tissue to plasma ratios of insulin between a cholesterol-encapsulated insulin formulation (cholestosome-insulin) administered by oral gavage and by subcutaneous injection, both compared with the same insulin administered subcutaneously (s.c.) In healthy female Wistar rats.

This study was designed for comparison of insulin bioavailability from the formulations administered, which is defined by the Area under the curve (AUC) calculated as the ratio of Oral to IV over 24 hours following the dosing of the formulation,

Sixteen healthy female Wistar rats ranging from 246.5 to 292.8 g in body weight were distributed into three study groups. Two 6-rat cohorts were dosed with cholestosome insulin oral and SC injection and the remaining 4 rats were given subcutaneous regular insulin as controls. Each rat was dosed with 1.0 ml/animal of a solution identified by Sponsor with cage number and rat number only. Two groups of female Wistar rats were fasted, ranging in duration from 3 hr prior—to 1 hr post-dosing. Rats had free access to water at all times. Rats were allowed free access to food starting one hour after dosing. Rats were monitored daily for mortality and morbidity, including signs of hypoglycemia. After baseline blood was collected 30 min before dosing, blood samples of 500 ul were collected at 30 min, 1 h, 2 h, 4 h, 6 b, 8 h, 10 h, 12 h and 24 hr for plasma preparation. All rats were euthanized 24 h post-dosing by CO₂ overdose. Immediately after euthanasia, a final blood sample was collected for plasma preparation by cardiac puncture. All plasma samples were frozen at −80° C. until assay using ELISA.

Animals were provided a commercial rodent diet (5% 7012 Teklad LM-485 Mouse/Rat Sterilizable Diet, Harlan) and sterile drinking water. All animals were confined to a limited access facility with environmentally-controlled housing conditions throughout the entire study period. The facility was maintained at 18-26° C., 30-70% air humidity, with a 12 h light-dark cycle (lights on at 6:00, off at 18:00). The animals were acclimatized in the housing conditions for at least 3-5 days before the start of the experiment.

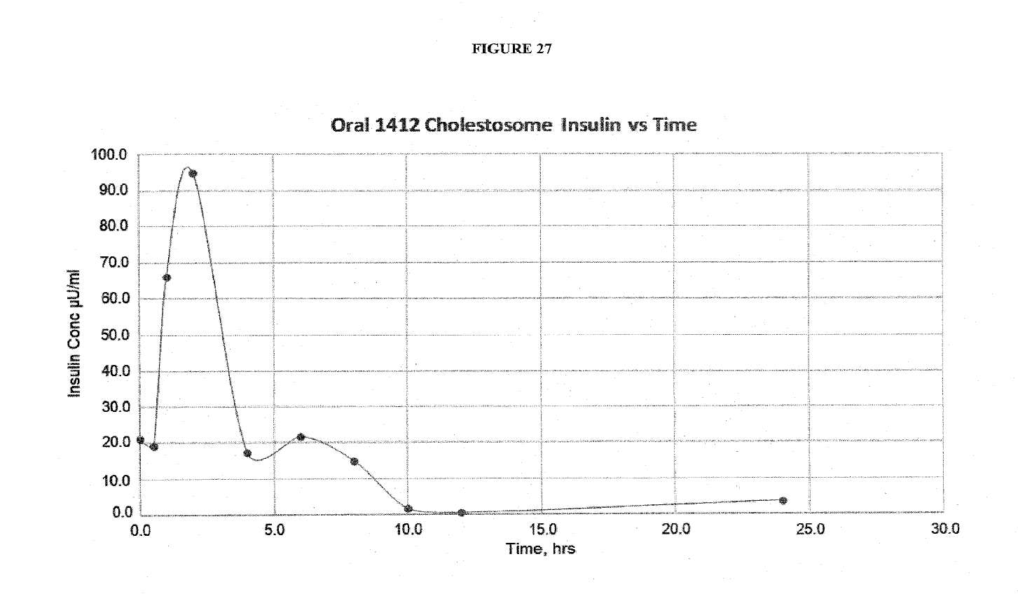

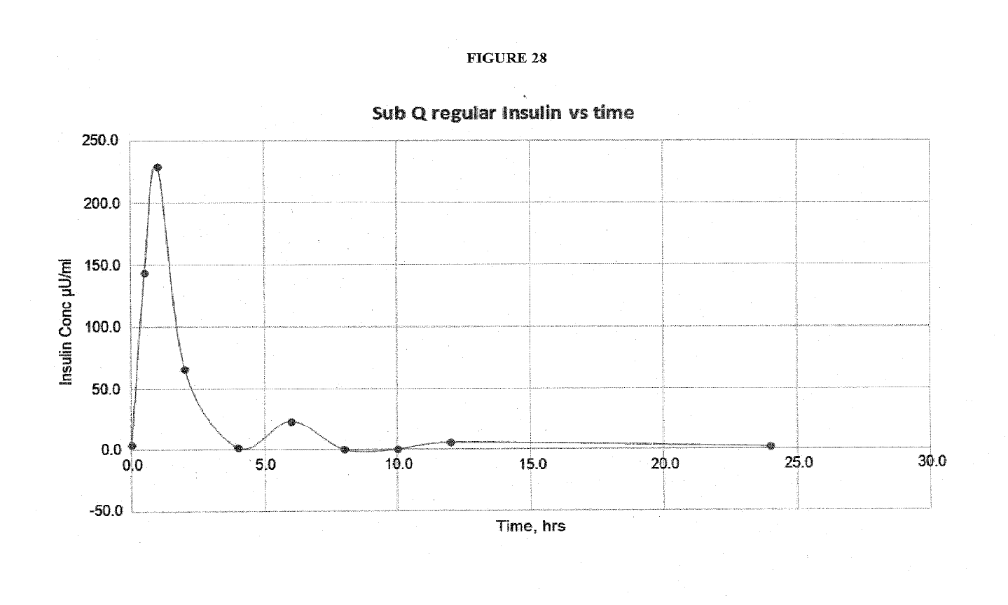

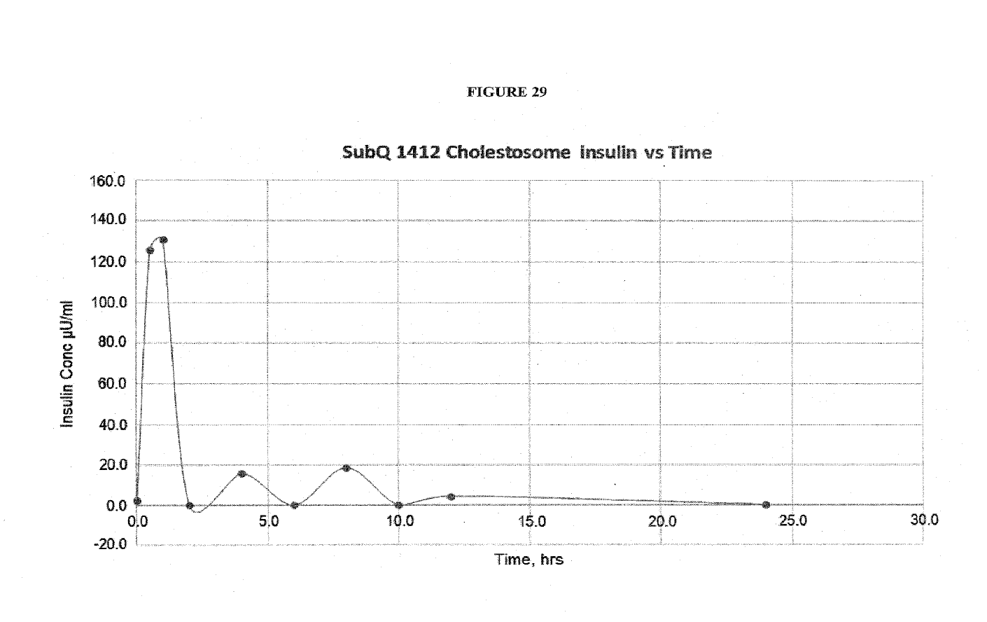

All animals behaved normally throughout the course of the study, and both oral and SC administration of cholestosome-encapsulated insulin and SC administration of regular insulin were well tolerated. No signs of drug toxicity or severe hypoglycemia were observed. Results. Plasma Insulin was assayed by ELISA (Mercodia) in triplicates, with a full standard curve and control samples on each assay day. Insulin concentrations were plotted as mean of time points, and the resulting blood concentrations and AUC values are shown in FIG. 27 (cholestosome oral, mean AUG 295) in FIG. 28 (S.C. regular insulin, mean AUG 344) and FIG. 29 (S.C. Cholestosome insulin, mean AUG 322). All AUG values were corrected for actual dose given (subtracting residual returned) and expressed as AUG to a dose of 0.25 units, which was approximately 1 unit/kg of body weight. The comparison of AUC from oral cholestosome insulin to S.C. insulin was considered to be an accurate estimate of relative oral bio-availability. In this experiment, oral bioavailability of cholestosome insulin was 86% vs SC injection and the relative bioavailability of oral cholestosome insulin vs SC cholestosome insulin was 92%.

### Tissue to Plasma Ratios Rat Studies

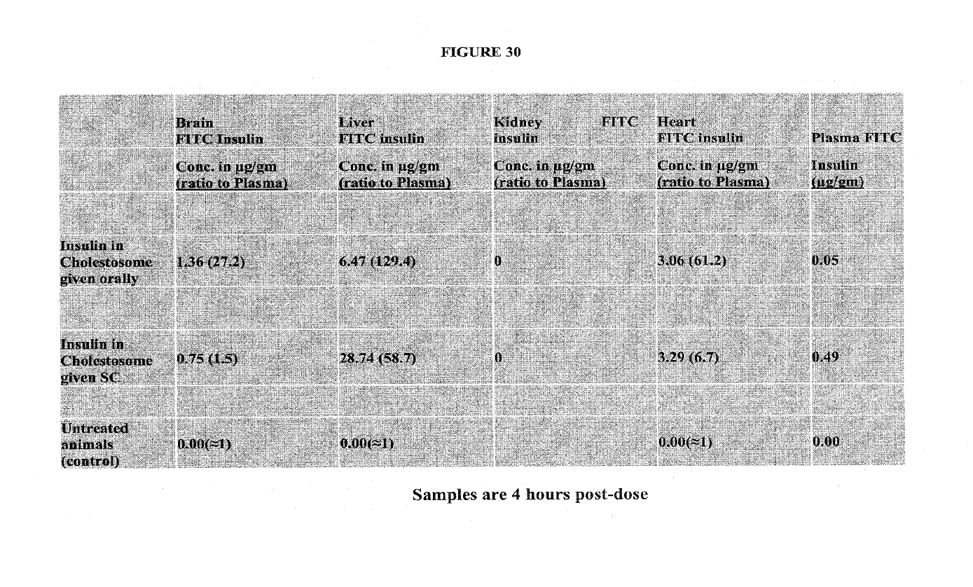

As shown in FIG. 30, cholestosome encapsulated FITC insulin shows high absorption into rat tissues after oral use, and in fact the concentrations in target tissues such as Brain, Liver, Kidney and Heart are higher after oral dosing than after the same dose given SC. This is expected because oral use places the FITC-insulin into chylomicrons and chylomicrons presumably load cells better than cholestosomes that are injected.

The assays here were detecting FITC according to a FITC calibration standard applied to tissue. Samples of tissues were taken 6 hr after dosing, at a time when plasma concentrations of insulin are low according to the data in FIGS. 26-28. Accordingly, assays of FITC reflect intracellular FITC since with these low values, blood and, presumably Interstitial fluid contamination of the tissues is unlikely to be a factor.

Organ concentrations of FITC in rats were similar to organ concentrations in mice. Kidney was different between the species, and liver was higher in rats than mice. However, overall tissue to plasma ratios were lower in rats than in mice. This reflects the higher plasma concentrations, particularly in the rats given SC dosing. Liver was the highest for rats and kidney the lowest. Brain distribution was better in mice than in rats. In summary, at a time when FITC and recombitant human insulin was nearly undetectable in plasma, the ratio of Tissue to Plasma remained very high. As insulin is rapidly metabolized by cells, these ratios reflect retention of intracellular FITC, and are entirely consistent with the long retention of FITC in our cell experiments, where the concentration may still be high 24 hr after exposure to FITC cholestosome insulin in the media.

### Example 6

### Protease Inhibitors to Add to Cholestosome Formulations

This example discloses a means of protecting cholestosome payload released peptides and proteins that are inside the cell membrane, from metabolism inside the cell. Said delay in intracellular metabolism serves to prolong their actions and thereby cause an increase in potency. Optionally said inhibitor of peptide metabolism may encourage a greater amount of the payload to be removed by the cell using its own exocytosis pathways.

A pilot study was performed whereby was added to MCF -7 cells that were also exposed to 1117 insulin-cholestosomes. It was found that MG-132 caused the likely exocytosis of more intact insulin-cholestosomes, indicating action on the enzyme cholesteryl ester hydrolase that otherwise acts to release insulin from cholestosomes.

In this embodiment of the present invention, said cholesteryl ester vesicles produced in the present invention and encapsulating one or more peptides additionally and optionally incorporates one or more of a protease inhibitor substance that slows the rate or entirely prevents the metabolism or catabolism of said peptides inside the cells of said mammal or patient or subject.

In another embodiment, said protease inhibitor is a trypsin inhibitor such as but not limited to: Lima bean trypsin inhibitor, Aprotinin, soy bean trypsin inhibitor (SBTI), or Ovomucoid.

In another embodiment, said protease inhibitor is a Cysteine protease inhibitor, where Cysteine protease inhibitors of the invention comprise: cystatin, type 1 cystatins (or stefins), Cystatins of type 2, human cystatins C, D, S, SN, and SA, cystatin E/M, cystatin F, type 3 cystatins, or kininogens.

In another embodiment, said inhibitor is the antibiotic substance bacitracin, where the effective amount of said substance is below that which causes toxic injury to cells after cholestosome delivery.

In another embodiment, said inhibitor is a DPP-IV inhibitor, for example but not limited to sitagliptin, saxagliptin, or linagliptin, each of which is commercially available for the purpose of prolonging the action of GLP-1 agonists.

In another embodiment, said inhibitor is an insulin degrading enzyme inhibitor (IDE inhibitor) including but not limited to ML-345 and others previously disclosed (18)

In another embodiment, said protease inhibitor is a Threonine protease inhibitor, where Threonine protease inhibitors of the invention comprise: MLN-519, ER-807446, TMC-95A.

In another embodiment, proteasome inhibitors such as Bortezomib or Ixazomib may be used for the purpose of prolonging the action of peptides delivered into cells using the present invention. In normal cells, the proteasome regulates protein expression and function by degradation of ubiquitylated proteins, and also cleanses the cell of abnormal or misfolded proteins.

In another embodiment, said protease inhibitor is an Aspartic protease inhibitor, where Aspartic protease inhibitors of the invention comprise: Pepstatin A, Aspartic protease inhibitor 11, Aspartic protease inhibitor 1, Aspartic protease inhibitor 2, Aspartic protease inhibitor 3, Aspartic protease inhibitor 4, Aspartic protease inhibitor 5, Aspartic protease inhibitor 6, Aspartic protease inhibitor 7, Aspartic protease inhibitor 8, Aspartic protease inhibitor 9, Pepsin inhibitor Dit33, Aspartyl protease inhibitor, or Protease A inhibitor 3.

In another embodiment, said protease inhibitor is a Metalloprotease inhibitor, where Metalloprotease inhibitors of the invention comprise: Angiotensin-1-converting enzyme inhibitory peptide, Anti-hemorrhagic factor BJ46a, Beta-casein, Proteinase inhibitor CeKI, Venom metalloproteinase inhibitor DM43, Carboxypeptidase A inhibitor, smpl, IMPI, Alkaline proteinase, INN, Latexin, Carboxypeptidase inhibitor, Anti-hemorrhaeic factor HSF, Testican-3, SPOCK3, TIMP1, Metalloproteinase inhibitor 1, Metalloproteinase inhibitor 2, TIMP2, Metalloproteinase inhibitor 3, TIMP3, Metalloproteinase inhibitor 4, TIMP4, Putative metalloproteinase inhibitor tag-225, Tissue inhibitor of metalloprotease, WAP, katal, immunoglobulin, or kunitz and NTR domain-containing protein 1.

In some embodiments, said protease inhibitor is a suicide inhibitor, a transition state inhibitor, or a chelating agent. In some embodiments, said protease inhibitor of the present invention is: AEBSF-HCl, (epsilon)-aminocaproic acid, (alpha) 1-antichymotypsin, antipain, antithrombin III, (alpha) 1-antitrypsin ([alpha] 1-proteinase inhibitor), APMSF-HCl (4-amidinophenyl-methane sulfonyl-fluoride), sprotinin, benzamidine-HCl, chymostatin, DFP (diisopropylfluoro-phosphate), leupeptin, PEFABLOC.®SC (4-(2-Aminoethyl)-benzenesulfonyl fluoride hydrochloride), PMSF (phenylmethyl sulfonyl fluoride), TLCK (1-Chloro-3-tosylamido-7-amino-2-heptanone HCl), TPCK (1-Chloro-3-tosylamido-4-phenyl-2-butanone), pentamidine isethionate, pepstatin, guanidium, alpha2-macroglobulin, a chelating agent of zinc, or iodoacetate, zinc. In some embodiments, said chelating agent is EDTA.

Each protease inhibitor substance disclosed herein, in an effective amount, represents a separate embodiment of the present invention, provided that the metabolism of said cholestosome released protein or peptide is delayed by said protease inhibitor. It will be obvious to one skilled in the art that any protease inhibitor that delays or prevents the metabolism of said peptide inside cells is preferred within the art of selection of protease inhibitors.

It is a preferred embodiment of the invention to employ a protease inhibitor that does not injure cells that release said protease inhibitor from the cholestosomes that reach the cytoplasm.

Each amount of a first or a second protease inhibitor represents a separate embodiment of the present invention, and it will be obvious to one skilled in the art that the amount of a selected protease inhibitor will delay or prevent the metabolism of said peptide or protein.

### Example 7

### Oral Trastuzumab Cholestosomes and IgG Control

Introduction, Breast cancer develops in glands (lobular carcinoma or milk ducts (ductal carcinoma) and is due to uncontrolled breast cell division. The Human Epidermal Growth Factor Receptor 2 (HER2) gene is responsible for the production of HER2 proteins. These proteins act as receptors on normal, healthy breast cells and aid in the growth, division, and repair of these cells. Trastuzumab (Herceptin®) is an IgG1 monoclonal antibody that has been proven to be therapeutic to HER2 positive breast cancer patients. Trastuzumab working with mitogen-activated protein kinase (MAPK) and phosphatidylinositol 3 kinase (P 13K) pathways interferes with the intracellular signaling of the HER2 receptor.

Many monoclonal antibodies with high therapeutic value for diseases such as cancer, tend to denature or form aggregates when exposed to the shear stresses of encapsulation. Trastuzumab has proven to be resistant against these shear stresses making its encapsulation promising for the future treatment of the typically Trastuzumab-resistant HER2-positive breast cancer patients.

The use of Cholestosomes as a method of delivery for Trastuzumab into HER2 negative MCF-7 human cells is conducted for evaluation of toxicity effects. Some in vitro models of HER2 positive cancer cell lines that appear responsive to Trastuzumab and can be evaluated in future studies are SKBR3 and MDA-MB-453.

Post in vitro encapsulation, oral and intracellular delivery of Trastuzumab its vivo is the ultimate goal. Currently, a safe and effective oral formulation is needed for Trastuzumab. Cholestosome technology shows the potential satisfy this need. As a novel drug delivery system. Cholestosomes show the ability to survive the acidic environment of the GI tract and enter the circulatory system via intestinal absorption. In this study, Trastuzumab has been successfully encapsulated in Cholestosomes.

### Preparation of Trastuzumab Stock Solution

A research formulation of trastuzumab for injection (hereafter, trastuzumab; ˜1 mg/mL trastuzumab, ˜5 mM histidine, ˜50 mM α-trehalose, ˜0.01% Tween® 20 and ˜1% w/v benzyl alcohol, pH6.0: generous gift from Kentucky Bioprocessing, Owensboro, Ky.) was used as starting material. An aliquot was reserved for later analysis. The trastuzumab needed to be concentrated before encapsulation, after detergent removal. Tween® 20 was removed from approximately 250 mL of trastuzumab using DetergentOUT™ Tween® Medi, (GBiosciences, St. Louis, Mo.). The resulting solution was made 2% in glycerol, concentrated by lyophilization and dialyzed against 4L of 0.5× PBS for 2 h (100 kD MWCO Spectra/Por® CE membrane; Spectrum, Rancho Dominguez, Calif.).

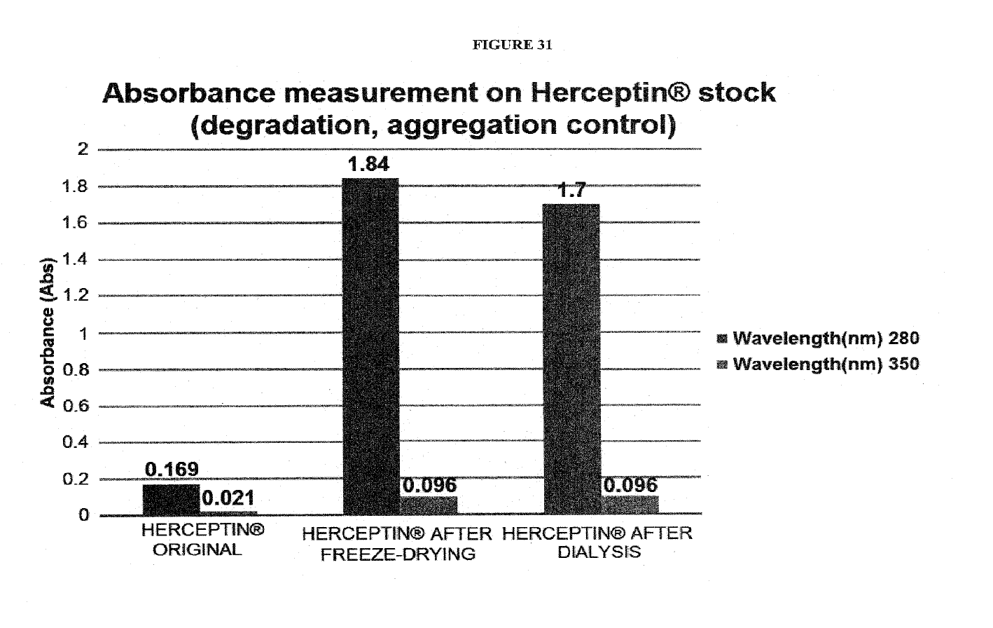

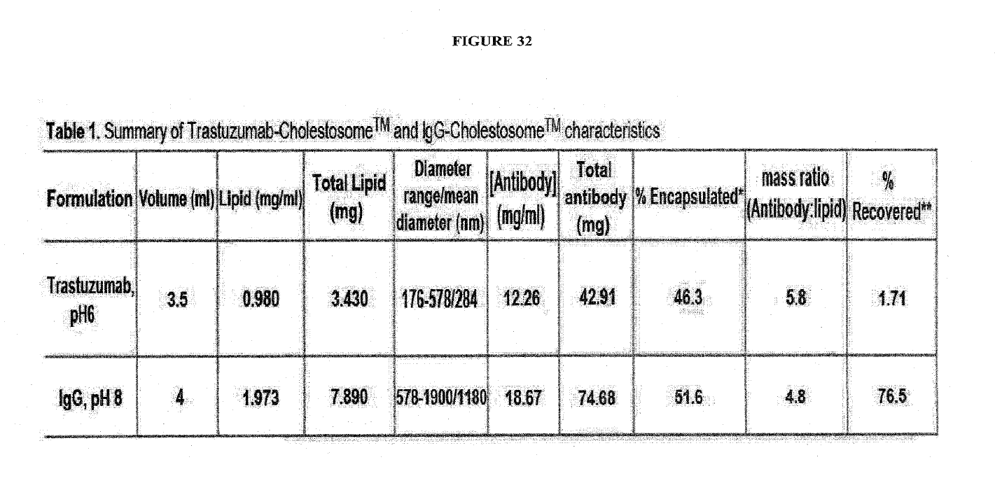

The Trastuzumab post-column solution was collected in 50 ml conical tubes. 80% Glycerol was added to the Trastuzumab post-column solution such that the final concentration of Glycerol was 2% in Trastuzumab post-column solution, which were then lyophilized. Absorbance of trastuzumab solution after lyophilization process was taken at 2 wavelengths: 280 nm and 350 nm using JENWAY spectrophotometer. Absorbance measurements are plotted in FIG. 31. A dialysis process was then performed on 20% Glycerol trastuzumab solution against 4 liters of 0.5× PBS for 2 hours using SPECTRUM Spectra/Por® Biotech Cellulose Ester (CE) Dialysis membrane with a molecular weight cutoff of 100,000 with nominal flat width 31 mm. The absorbance measurements of trastuzumab post-dialysis solution are recorded in FIG. 32. The final trastuzumab stock solution for encapsulation contains approximately 12.52 mg/ml trastuzumab, 0.18% benzyl alcohol pH 6.0, 0.428 mM Histidine HCl, 0.346 mM Histidine and 8.741 mM Trehalose dihydrate.

### Preparation of Trastuzumab-Cholestosomes:

5 mL of Trastuzumab stock solution was added into a 15 ml conical tube and equilibrated at 40° C., 80 mg of Cholesteryl Myristate and 75 mg of Cholesteryl Laurate (NU Check Prep; Elysian, Minn.) were added into a 100 ml round-bottom flask (RBF) and solubilized with 5 ml of diethyl ether. The solution was then put on a Buchi Rotovapor R-3 to spin at speed setting 4 for 10 minutes under no vacuum, and then exposed to low vacuum for 10 minutes at 40° C. The RBF was then removed and 5 ml Trastuzumab stock solution at 40° C. was then added to the RBF and sonicated at 40° C. for 20 minutes. RBF was rotated during sonication every 5 minutes. Trastuzumab Cholestosomes were then filtered through a sterile cell strainer 40 μm mesh basket (ThermoFisher Scientific, Waltham, Mass.) to remove excess lipid materials and stored at 4° C.

### Separation of Unencapsulated Trastuzumab

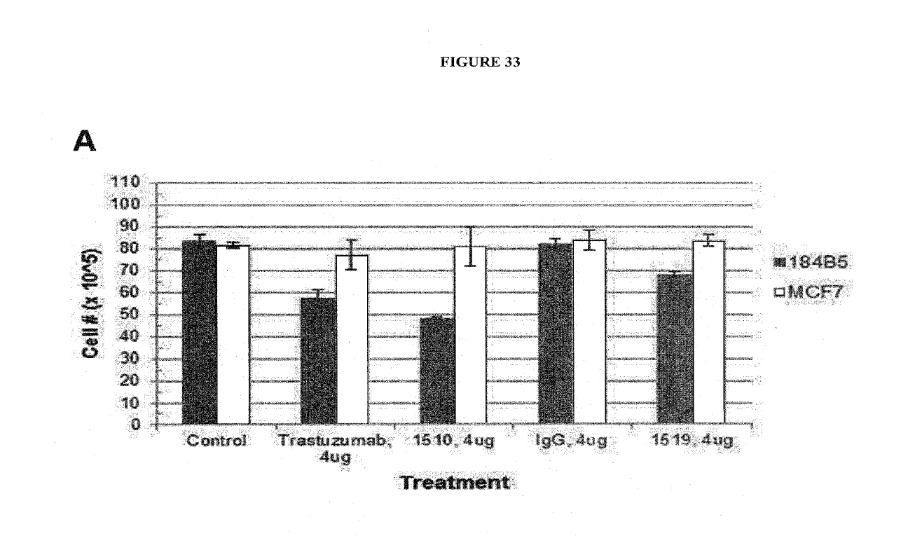

Unencapsulated Trastuzumab was separated using Protein G-Sepharose (Biovision Inc.; Milpita, Calif.) according to the manufacturer's instructions. Briefly, the protein G matrix was washed 5 times with binding buffers, loaded with Trastuzumab-Cholestosomes and incubated at room temperature for 90 minutes. The samples were then spun at 5000× g for 30 seconds. The supernatant was removed using a micropipette and saved for further analysis. Elution butlers were then added to the matrix and the sample was centrifuged as previously. Supernatants were collected and saved for further analysis. Unexpectedly, difficulty was encountered in recovering trastuzumab-Cholestosomes from the protein G-sepharose used to remove the unencapsulated trasturumab. Empirical evidence (FIG. 33 and not shown) suggests this may he related to the amount of lipid in the formulation. Efforts are ongoing to find ways to maximize Cholestosome recovery during the separation process.

### Preparation of IgG-Cholestosomes:

5 mL of an 18.8 mg/ml Human IgG stock (0,02M Na₂HPO₄, 0.15M NaCl; Innovative Research; Novi, Mich.) was used to prepare IgG-Cholestosomes using the method described above. Unencapsulated IgG was separated from IgG-Cholestosomes using protein G-sepharose as described above for trastuzumab-cholestosomes.

### Analysis of Trastuzumab Cholestosomes and IgG Cholestosomes

Dynamic light scattering (DLS) analysis of Cholestosomes was performed with the Nanobrooks −90 Plus PALS. Analysis of lipid concentration were obtained using the HPLC (Shimadzu 2020) Spectrophotometry was performed on the Cholestosome™ solutions as described above.

Cell treatments; MCF-7 cells were cultured in 75 cm² flasks and incubated in a 37° C. and 5% CO₂ environment until 80-90% confluent. Incubation of MCF-7 cells with various treatments and formulations was performed in a 24-NveII format. Cells were seeded at 5×10⁵cells/well and incubated 24 hours before treatment. Cells were then incubated for 24 hr, then washed, trypsinized and counted in a hemocometer. Viability was determined by trypan blue exclusion

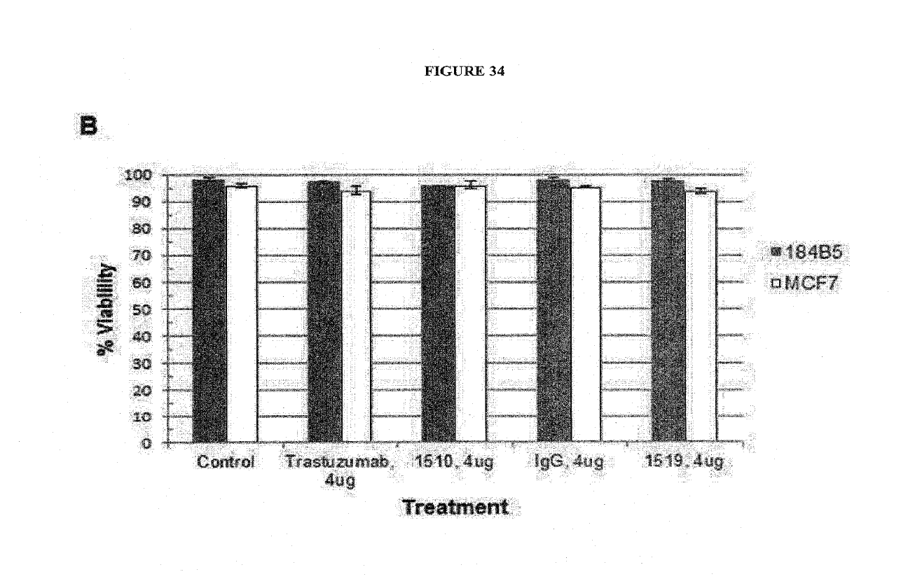

Crude Cholestosomes were initially put on breast epithelial cells (184B5 and MCF-7) to test for effects on cell growth FIG. 33 and viability FIG. 34. Neither of the soluble or Cholestosome formulated antibodies had any effect on viability of the cells. Cholestosomes formulated trastuzurnab and to a much lesser extent, IgG affected 184B5 cell growth, but it did not affect MCF-7

### Results and Discussion

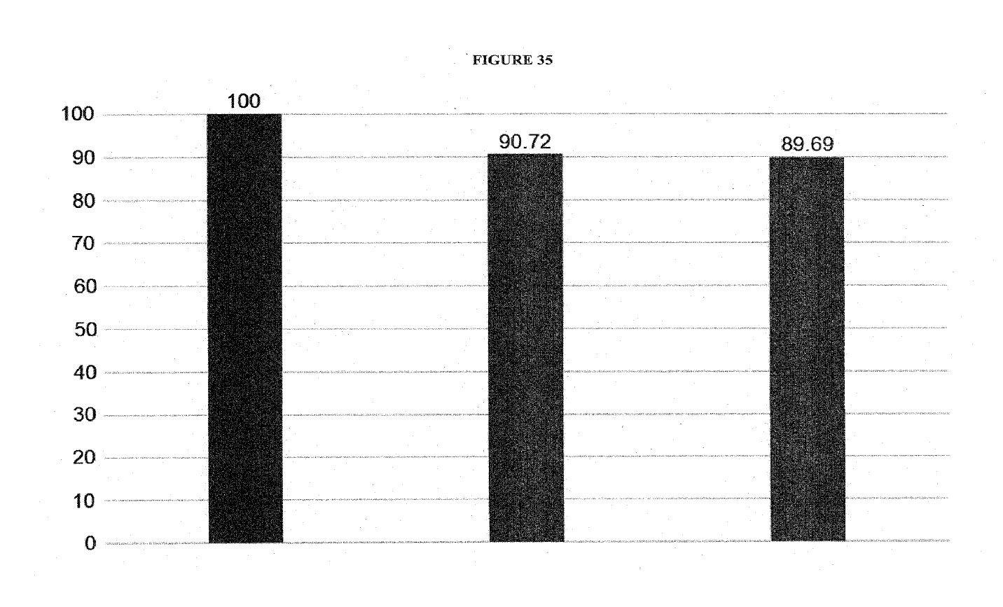

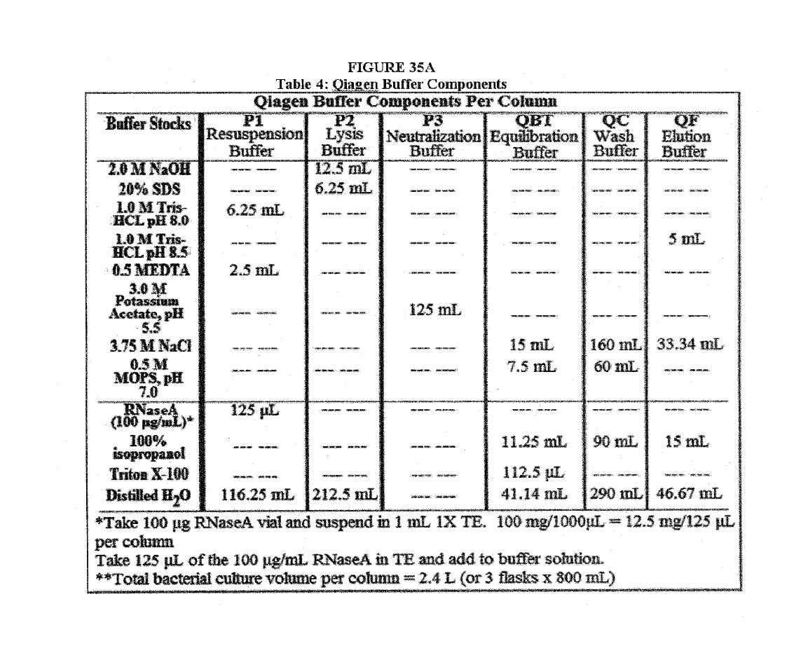

Typical clinical formulations of protein biologics have lower amounts in solution, necessitating concentration of the protein before undertaking of Cholestosome encapsulation. In some cases, there has been substantial losses due to aggregation during the concentration process such as lyophilization. FIG. 35 suggests that for trastuzumab, this mode of loss is minimal, as trastuzumab has not shown any degradation or aggregation under the stress of lyophilization or encapsulation. FIG. 35 shows that losses are minimal for both IgG and Trastuzumab in Cholestosomes, as both can be successfully measured without any lipid interferences at 1000 fold dilution.

Cholestosome formulations encapsulating IgG and trastuzumab had differences in lipid incorporation and sizes, but were similar in the amounts estimated to be encapsulated and in the antibody to lipid mass ratio. Trastuzumab has been successfully encapsulated into Cholestosomes, with a loading ratio w/w of approximately 50%. Neither encapsulated trastuzumab nor IgG showed any toxicity to MCF-7 cells or 184B5 cells, which indicates that these formulations could be tested in animal models.

### Example 8

### Cholestosome-FITC Loading of MCF-7 and ARPE19 Retinal Cells

FITC cholestosomes were also prepared from a 1:1 molar mixture of Cholesteryl myristate (C14) and Cholesteryl palmitate (C16). Surprisingly rapid uptake of FITC into MCF-7 cells was observed from this cholestosome preparation.

Preparation of FITC stock: A solution of 1.0 mg/ml FITC solution was made at neutral pH in 1× PBS.

### Preparation of Cholestosomes Using Cholesteryl Myristate/Palmitate:

A 1:1 Molar solution of cholesteryl myristate (75 mg) and cholesteryl palmitate (84 mg) was made in 5 ml of chloroform. The chloroform solution was added to a round bottom flask attached to a Buchi Rotoevaporator. The flask was placed in a water bath set at 65 C and rotated for 10 minutes at Which point the vacuum was applied to remove the organic solvent. Once the solvent was removed 5 ml of a 1 mg/ml aqueous FITC solution was added. The solution was moved to a sonicator bath that readied a high temperature of 57 C. Sonication was carried out for 20 minutes. The formulation was then filtered to separate the formulation from the unused lipid.

Separation of unencapsulated FITC. The formulation was gravity filtered through an 0.22u filter ensuring that the volume removed was replaced by fluid at regular intervals.

Cell studies-MCF-7

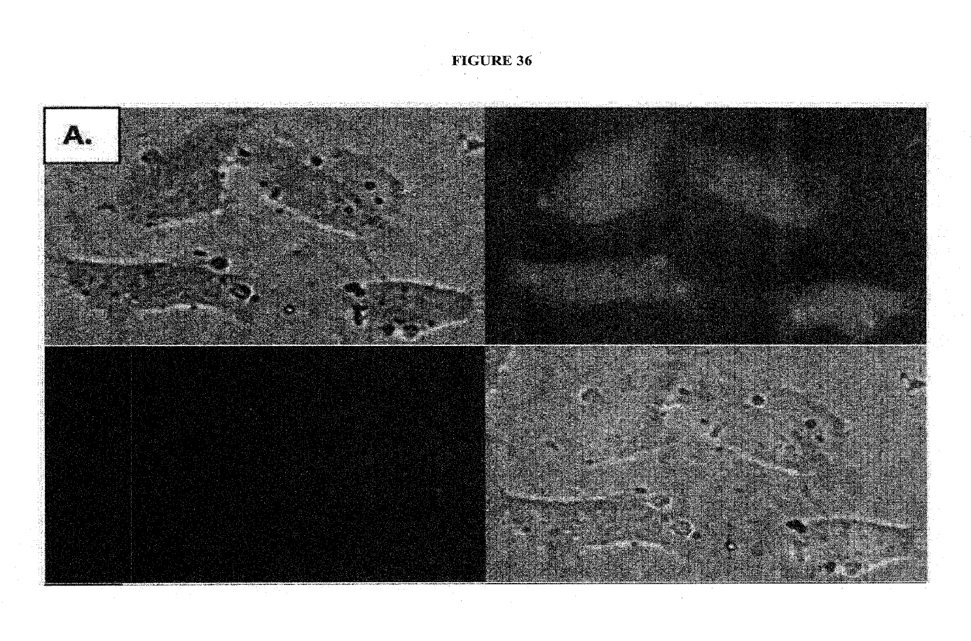

In this experiment, 22 ug of FITC formulation was placed on MCF-7 cells, Fluorescent imaging was performed over time, with images taken at 2, 4, 8 and 24 hours. The data revealed a more rapid uptake for FITC cholestosomes prepared from Myristate and Palmitate compared with FITC cholestosomes prepared from Myristate and laurate, as shown in FIG. 36. The difference in rate of uptake was unexpected and disclosed herein as a special property of cholestosome construction. In this particular experiment, there was also uptake of FITC itself into MCF-7 cells, while it was clear that there was much greater fluorescence from cholestosome encapsulated FITC compared with FITC alone.

### Cell Studies-Retinal Cells ARPE19

Cholestosomes made from cholesteryl myristate and cholesteryl palmitate an a 1:1 Molar ratio loading human retinal epithelial cells with FITC is shown in FIG. 36. In this experiment, 22 ug of FITC formulation was placed on ARPE19 cells. Fluorescent imaging was performed over time, with images taken at 2, 4, 8 and 24 hours. Panel A shows images of ARPE-19 cells treated for 2 h with FITC-Cholestosomes made from myristate/palmitate. The top left panel is the phase contrast image; the top right is the green fluorescent channel image at 2 hr; the bottom left panel is the red fluorescent channel image; the bottom right panel is the merged image. The data suggested that there was a more rapid uptake for FITC cholestosomes prepared from Myristate and Palmitate compared with FITC cholestosomes prepared from Myristate and laurate. The difference in rate of uptake was unexpected and disclosed herein as a special property of cholestosome construction.

### Example 9

### Nucleic Add Cholestosomes: GFP Plasmid

Fluorescent proteins are genetically encoded tools that are used extensively by life scientists. The original green fluorescent protein (GFP) was cloned in 1992, and since then scientists have engineered numerous GFP-variants and non-GFP proteins that result in a diverse set of colors. Successful transfection of Green Fluorescence Plasmid (GFP) and expression of green fluorescence by the transfected cells is widely considered to be definitive evidence of a successful intracellular delivery mechanism. Accordingly, these experiments were conducted in order to demonstrate intracellular delivery of nucleic material using cholestosomes encapsulation.

Steps in the growth and purification of GFP, beginning with a stock of pgWizGFP plasmid as described by Himoudi-2002 (22) were as follows:

### Preparation of LB Miller Broth

1. 800 mL of H₂O was added to a 2 L Erlenmeyer flask.

2. 20 g LB Miller broth (Sigma L3522) was dissolved by swirling until liquid was a clear yellow and all powder clumps were dissolved. Steps 1 and 2 were repeated twice more to obtain 3 flasks, each containing 800 mL of LB Miller Broth.

3. Flasks were autoclaved on liquid media setting

4. The sterilized LB broth was brought to 50 mg/mL Kanamycin sulfate (GIBCO, 100×, 151600-054) and inoculated with a glycerol stock of pgWizGFP plasmid (Aidevron, Fargo, N.D.; 5757 bp).

5. Flasks were incubated with shaking (200 rpm) overnight at 37° C.

Column Purification of pgWizGFP:

1. Buffers P1, P2, and P3 (Plasmid Giga. Kit, Qia gen Corp Germantown, Md.) were prepared according to the manufacturer's specifications (buffer composition specifications are provided in Table 4).

2. Approximately 22-24 hours post incubation, culture growth was monitored by measuring the optical density (OD) at 600 nm using an appropriately blanked Spectrophotometer until cultures had between 1.5E+9 and 3-4E+9 bacteria,

3. Bacteria were pelleted at 6,000×g, 15′, 4° C., (JLA 9.1000 rotor; Avantit J-E Centrifuge Avanti, Beckman-Coulter, Brea, Calif.). The supernatant was discarded,

While bacteria were pelleting, 3 QIAGEN-tip 10000 columns were each equilibrated with 75 mL of Buffer QBT.

5. Bacterial pellets were resuspended in 125 mL Butler PI, combining all pellets.

6. 125 mL of Buffer P2. was added slowly to avoid foaming, it was then mixed thoroughly by vigorously inverting 6 times and finally, incubated at RT for 5′ on the benchtop.

7. 125 mL of chilled Buffer P3 was added to the resuspended pellet mixture, mixed vigorously by inversion 6 times and incubated on ice for 30 minutes.

8. Treated pellets were spun in bottles at 20,000×g, for 30 min, at 4° C. In a JLA 16.250 rotor in the same centrifuge as in Step 3

9. The supernatants were carefully decanted into clean 250 mL centrifuge bottles.

10. The supernatants were spun at 20,000×g, 15 min, and 4° C. with a JLA 16.250 rotor to get rid of any additional particulates which would clog the column if present.

11. The supernatant obtained from step 10 was applied to the column. The equilibration flow through was discarded.

12. The columns were washed with 600 mL of Buffer QC.

13. The plasmid was diluted with 100 mL of Buffer QF into a cleaned 250 mL centrifuge bottle.

14. The plasmid was precipitated by adding 70 mL of RT isopropanol to the 250 mL centrifuge bottle containing eluted plasmid and mixing well.

15. This mixture was centrifuged at 15,000×g, 30′, 4° C., using the JLA 16.250 rotor. The supernatant was discarded.

16. The pellet was rinsed with 10 mL RT 70% ethanol and spun again at15,000×g, 10 min, 4° C., JLA 16.250 rotor. The supernatant was discarded. Note: The pellet should look pure white.

17. The pellet was air dried for ˜20 min by inverting the centrifuge bottle over a paper towel at ˜45 degree angle. 2 mL nuclease free water was added to the pellet. After overnight incubation at 4° C., the pellet was resuspended and transferred to a 15 mL conical tube and then stored 4° C. The yield should be a between 10 mg and 15 mg from each 2.4 L total culture volume.

18. Using these methods, approximately 100 mg of pgWizGFP was prepared and used in the preparation of pgWizGFP-Cholestosomes™

Preparation of pgWizGFP -Cholestosomes™ from Cholesteryl Myristate/Cholesteryl Laurate)

5 mL of a 4 mg/mL the purified pgWizGFP stock solution was added to a 15 ml conical tube and equilibrated at 55° C. At the same time, 80 mg of Cholesteryl Myristate and 75 mg of Cholesteryl Laurate (NU Check Prep; Elysian, Minn.) were put into a 100 ml round bottom flask (RBF) and solubilized with 5 ml of diethyl ether. The flask with the solubilized esters was then put on a Rotovapor R-3 (Buchi, New Castle, Del.) to spin at speed setting 4 for 10 minutes at 55° C. without vacuum, and then was exposed to low vacuum for an additional 10 minutes. The RBF was then removed and the pre-equilibrated 4 mg/mL pgWizGFP stock solution was then added to the RBF and it was sonicated at 50° C. for 20 minutes (90% power, 35 kHz) in an Elmasonic P sonicator (Tovatech, Maplewood, N.J.). RBF was rotated during sonication every 5 minutes. The resulting pgWizGFP-Cholestosomes™ were then filtered through a sterile 40 μm nylon mesh strainer (ThermoFisher Scientific, Waltham, Mass.), In order to separate the unencapsulated FITC, the formulation then gravity filtered through an 0.22 u filter ensuring that the volume removed was replaced by fluid at regular intervals. The formulation was stored at 4° C. until analysis and use in cell studies.

Preparation of pgWizGFP Cholestosomes from Cholesteryl myristate/Cholesteryl palmitate,

5 ml of a 3.6 mg.ml the purified pgWizGFP stock solution was added to a 15 ml conical tube and equilibrated at 55° C. At the same time 75.5 mg of cholesteryl myristate and 83.5 mg of cholesteryl palmitate (NU Check Prep Elysian, Minn.) were put into a 100 ml round bottom flask (RBF) and solubilized with 5 ml of chloroform. The flask with the solubilized esters was then put on a Rotovapor R-3 (Buchi, New Castle, Del.) to spin at speed setting 4 for 10 minutes at 65° C. without vacuum, and then was exposed to low vacuum for an additional 10 minutes. The RBF was then removed and the pre-equilibrated 3.6 mg/mL pgWizGFP stock solution was then added to the RBF and it was sonicated at 57 ° C. for 20 minutes (90% power. 35 kHz) in an Elmasonic P sonicator (Tovatech, Maplewood, N.J.). RBF was rotated during sonication every 5 minutes. The resulting pgWizGFP-Cholestosomesmt were then filtered through a sterile 40 μm nylon mesh strainer (ThermoFisher Scientific, Waltham, Mass.) In order to separate the unencapsulated FITC, the formulation then gravity filtered through an 0.22 u filter ensuring that the volume removed was replaced by fluid at regular intervals. The formulation was stored at 4° C. until analysis and use in cell studies.

Analysis of pgWizGFP-Cholestosomes™:

Dynamic light scattering (DLS) analysis of Cholestosomes™ was performed with the Nanobrook 90 Plus PALS. (Brookhaven, Holtsville, N.Y.). Lipid concentration was determined using HPLC and comparison to the appropriate standard curve (LCMS 2020, Shimadzu, Columbia, Md.). DNA concentration was determined by measurement of absorbance at 260 nm using a BioSpec-nano spectrophotometer (Shimadzu, Columbia, Md.).

Intracellular delivery Studies and Imaging

In order to test the intracellular delivery properties of pgWizGFP-Cholestosomes™ (200 ul of a cholestosome encapsulated pgWizGFP preparation was added to the media of MCF-7 cells and the cells were tested for the presence of green fluorescent protein variants, as a means of monitoring gene transfer and expression in mammalian cells over time, in the manner of Cheng 1996 (23).

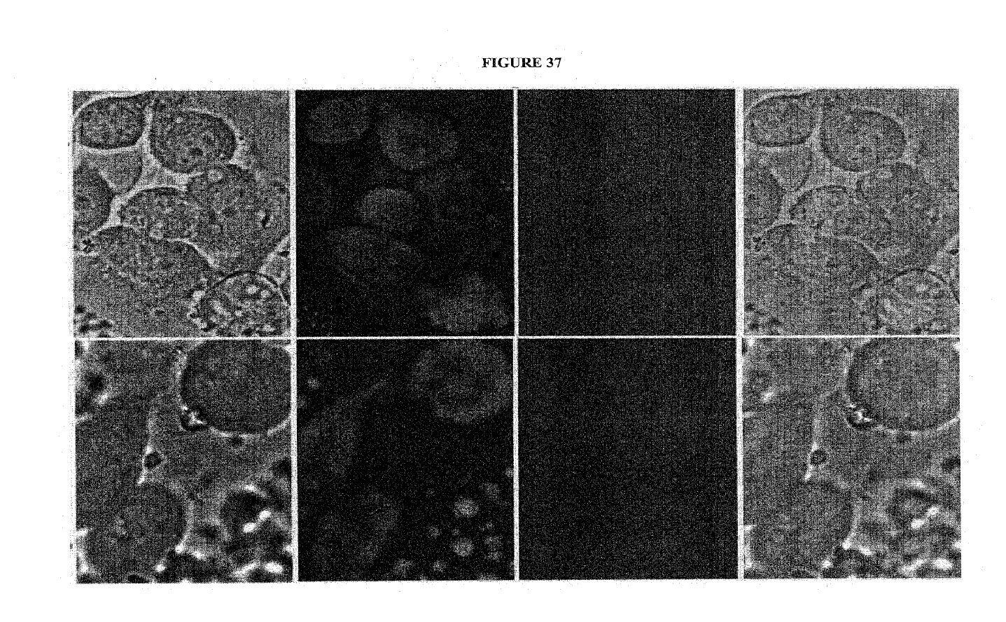

FIG. 37 Shown are images of MCF7 cells treated with pgWizGFP Cholestosomes made from Cholesteryl myristate/palmitate (top panel at 30 hr) and pgWizGFP Cholestosomes made from Cholesteryl myristate/laurate (bottom panel at 24 hr). In each panel, the far-left image is phase contrast, the next is green fluorescent channel, the next is red fluorescent channel, and the far right is the merged image. The images show that both formulations load. MCF7 cells with pgWizGFP plasmid, with expression of Green Fluorescence. Media control panels (not shown had negligible fluorescence, consistent with background autofluorescence. Overall, the images demonstrate surprisingly robust cellular uptake of pgWizGFP-Cholestosomes™ which was followed by evident replication behavior of the GFP plasmid inside these cells. Cells remained viable and showed no loss of integrity during, these GFP expression experiments.

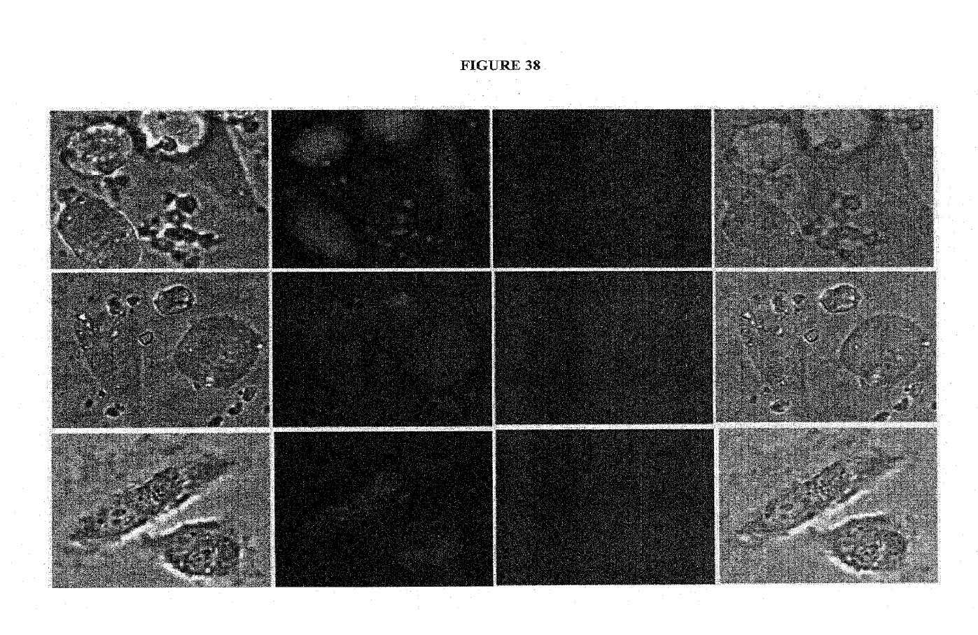

The readings of the cholesteryl myristatecholesteryl palmitate GFP Cholestosomes in FIG. 38 were taken 24 hr after addition to the media. Shown are images of ARPE-19 Human retinal cells treated for 24 h with pgWizGFP Cholestosomes made from Cholesteryl myristate/palmitate (top panel) and pgWizGFP Cholestosomes made from Cholesteryl myristate/laurate (middle panel) and pgWizGFP alone added to media (bottom panel). In each panel, the far-left image is phase contrast, the next is green fluorescent channel, the next is red fluorescent channel, and the far right is the merged image. The images show that both formulations load ARPE-19 cells with pgWizGFP plasmid, resulting in the expression of Green Fluorescence at 24 hr. pgWizGFP alone shows negligible fluorescence at 24 hr, indicating little spontaneous entry of plasmid into cells (not shown). Overall, the images demonstrate surprisingly robust cellular uptake of pgWizGFP-Cholestosomes™ which was followed by evident replication behavior of the GFP plasmid inside these cells. Cells remained viable and showed no loss of integrity during, these GFP expression experiments.

### Example 10

### Use of Cholestosomes in Cancer Immunotherapy

This example describes a completely personalized immunotherapy approach to patients with solid tumors. The full scope of the invention can be practiced when the tumor can be excised and tissue obtained for processing into an autologous immunotherapy composition which is purified and then encapsulated into cholestosomes, optionally with an adjuvant. Said construct may be administered by direct injection into said patient's tumor in one aspect of the present invention. In another preferred aspect, said composition may be loaded into a capsule and the capsule then emetically coated to release said composition at pH 7.3 to 7.6 after oral administration. When the invention is practiced in this manner, said composition is delivered only at the ileum and potentially the appendix cholestosome encapsulated immunotherapy compositions applied to the ileum are taken up by dendritic cells lining the ileum in Peyers Patches, and these dendritic cells program CD4+ and CD8+ cells to attack said cancer cells expressing the targeted cancer antigens at their body location. Examples include melanoma, lung cancers of all types, colon cancer, hepatocellular carcinoma, pancreatic cancer, ovarian cancer, head and neck cancer, prostate cancer, breast cancer and brain tumors such as glioblastoma.

### Exposure of Dendritic Cells to Antigens and Adjuvants

Dendritic cells (DCs) are the most effective antigen-presenting cells. In the last decade, the use of DCs for immunotherapy of cancer patients has been vastly increased. High endocytic capacity together with a unique capability of initiating primary T-cell responses have made DCs the most potent candidates for this purpose. Although DC vaccination given by injection occasionally leads to tumor regression, the responses to this approach have been modest. The present invention overcomes these limitations by encapsulating the active tumor composition in cholestosomes, thereby ensuring that it will reliably enter dendritic cells in the first aspect. In the second aspect, the dendritic cells to be targeted are those of lymphoid responsive tissues of Peyers Patches in the ileum of the distal intestinal tract.

A variety of approaches have been-used to deliver tumor-associated antigens (TAA) in conjunction with dendritic cells (DC) as cellular adjuvants, DC derived from monocytic precursors have been pulsed with whole tumor antigen using a variety of strategies and have been demonstrated to induce CD4+ and CD8+ antitumor responses. In the present study, monocyte-derived DC have been pulsed with lysate from an allogeneic melanoma cell line, A-375, and used to repeatedly stimulate T cells. The resultant T cells were examined for cytotoxic activity against A-375 targets as well as the HLA A2-positive melanoma cell line DFW. Uptake of FITC-labeled melanoma lysate by DC established that lysate of melanoma cells was efficiently endoeytosed. Stimulation with lysate-pulsed DC resulted in strong proliferative responses by T cells, which could be inhibited by antibodies against both MHC class I and class II.

T cells stimulated in vitro with lysate-pulsed DC demonstrated potent cytotoxicity against the melanoma targets which were blocked by antibodies against MHC class I. Lysate-pulsed DC also elicited IFN-gamma secretion by T cells as measured in an ELISPOT assay. We have also examined the ability of lysate-pulsed DC to present melanoma-associated antigens to T cells. ELISPOT assays with synthetic peptides of melanoma-associated antigens, such as gp100, magel, NY -ESO, and MART-1, revealed that lysate-pulsed DC could stimulate T cells in an antigen-specific manner. The results demonstrated that lysate from allogeneic tumor cells may be used as a source of antigens to stimulate tumor-specific T cells in melanoma.(24)

### Autologous Tumor Acquisition and Processing

In each case where a patient tumor specimen (an autologous sample) is submitted for processing into cholestosomes, the first step in tumor processing will be to firm a “Lysate”

The term “lysate” used in the specification means a state of dispersion of the solidified tumor material in an aqueous medium such as water, physiological saline, and a buffer solution to an extent that any solid mass cannot be observed with naked eyes, and to an extent that the dispersoids can be phagocytosed by the antigen-presenting cells. However, the term should not be construed in any limiting way. The details of the preparations of the fixed tumor materials, the preparations of the microparticles, and the preparations of lysates are specifically described in the examples of the present specification. Accordingly, those skilled in the art can prepare the desired microparticles or the lysates by referring to the above general explanations and specific explanations in the examples, and appropriately modifying or altering those methods, if necessary.

Tumor lysis and processing, of antigenic constructs in the lysate will precede separation of fractions using centrifugation. Fractions collected will each be tested as antigens against the T-cells of the patient. A suitable adjuvant would be lipopolysaccharide (LPS) in a non-limiting example. For analysis of T-cell responses in peripheral blood. PBMCs will be isolated via density gradient centrifugation, counted and re-stimulated by addition of peptide and syngeneic BMDC. There should be an increase in IL-2 and IFN gamma as a means of detecting responsiveness. Subtyping of T-cell responses will be performed with an MHC class II blocking antibody (20 mg/ml clone M5/114, BioXcell). All samples will be tested in duplicates or triplicates.

Fractions of said patient's tumor lysate which demonstrate T-cell responsiveness will be processed by encapsulation into cholestosomes. These active fractions, processed into cholestosomes, will he combined with LPS adjuvants and prepared for oral delivery to the patient who was the source of said tumor.

In all cases, a blood sample suitable to isolate PBMCs will be taken at the same time as the tumor is excised. Said blood sample will be used to test in vitro responses to the intact tumor and its components as processed in the laboratory as antigens.

### Allogenic Tumor Acquisition and Processing

For patients without an accessible tumor for preparation as stated herein, the procedure will be conducted using the patients T-cells harvested from a blood specimen, and either an allogenic tumor of the same type will be processed for antigens (starting as a tumor lysate as done with autologous tumor), or the patients Tcells will be tested against known “common antigens” for the tumor type, which may consist of gp100 for melanoma, NY-ESO-1 for ovarian or hepatocellular carcinoma, or others as disclosed in the art.

T cells stimulated in vitro with lysate-pulsed DC demonstrated potent cytotoxicity against the melanoma targets which were blocked by antibodies against MHC class 1. Lysate-pulsed DC also elicited IFN-gamma secretion by T cells as measured in an ELISPOT assay. We have also examined the ability of lysate-pulsed DC to present melanoma-associated antigens to T cells. ELISPOT assays with synthetic peptides of melanoma-associated antigens, such as gp100, magel, NY-ESO, and MART-1, revealed that lysate-pulsed DC could stimulate T cells in an antigen-specific manner. The results demonstrated that lysate from allogeneic tumor cells may be used as a source of antigens to stimulate tumor-specific T cells in melanoma.(24)

For purposes of the invention, any antigen considered reactive with the patient's own T-cells in this testing procedure may be suitable for preparation in cholestosomes, as long as those cholestosomes will elicit an immune response from the patient's T cells once prepared in cholestosomes. The ELISPOT assay may be used to identify cytokines that result from antigen recognition by the patient's T cells.

ELISPOT assay example: IFN-γ ELI SPOT kit (Dakewei, China) was used to determine the frequency of cytokine-expressing cells after overnight activation with peptides. Briefly, T cells (105 per well) and peptides (50 μg/ml) were added to duplicate wells and DCs were added at ratio (DC:T) of 1:5˜1:10 for 18˜20 h. The plates were washed before the addition of the diluted detection antibody (1:100 dilution) and then incubated for 1 h in 37° C. After washing the plates, streptavidin-AP (1:100 dilution) was added and incubated at 37° C. for another 1 h. AEC solution mix was then added to each well, and the plates were left in the dark for about 15˜25 m at room temperature before deionized water was added to stop development. Plates were scanned by Elispot CTL Reader (Cell Technology Inc, Columbia, Md.) and the results were analyzed with Flispot software (AID, Strassberg, Germany).(25)

### Additonal Means of Acquisition of Tissue Compositions for Use in the Invention

For patients without an accessible tumor for preparation as stated herein, the procedure will be conducted using the patients T-cells harvested from a blood specimen, additionally, tested against exosomes taken from the blood of said patient. The source of these cancer exosomes may be the patient's tumor grown in vitro, followed by harvesting of the supernatant and isolation of exosomes. Optionally, the exosomes may be collected directly from the patient's blood, using a blood concentration technique such as the Aethlon Hemopurifier (Aethlon Medical, San Diego, Calif.) (26)

For patients with an accessible tumor and where there is rapid access to Whole Genome sequencing, the procedure will be used to derive a poly-neo-epitope mRNA cancer immunotherapy antigen construct for use in the cholestosome delivery vesicle. Tumor-specific mutations are ideal targets for cancer immunotherapy as they lack expression in healthy tissues and can potentially be recognized as neo-antigens by the nature T-cell repertoire. Every patient's tumor possesses a unique set of mutations (‘the mutanome’) that must first be identified by whole genome sequencing. The neoantigens are processed into an injectable vaccine construct which does work well in mice. This approach is undergoing pilot phase I human testing and is considered an advance in treatment based on “just in time” production of a single patient vaccine/immunotherapy. The novel aspect of this approach is to encapsulate the construct in cholestosomes, add an adjuvant, then deliver this construct to the ileum in order to directly pre gram said patient's dendritic cells in Peyers Patches. This approach is revolutionary and rests on the unique property of cholestosome encapsulated antigens of all type to be taken directly into dendritic cells that monitor the distal intestine in search of antigenic material.

The approach proposed here ideally begins with a resected autologous tumor and integrates advances in the field of next-generation sequencing, computational immunology and synthetic genomics to define the broad based neo-epitope target repertoire specific to that tumor. Targeting multiple mutations at once may in theory pave the way to solve critical problems in current cancer drug development such as clonal heterogeneity and antigen escape(27)

This construct being injected in the current phase I trial. According, it is believed by the inventors that cholestosome delivery to dendritic cells via the oral delivery to the ileum would be entirely novel in terms of improved safety and of most importance, by greatly expanding the chances for successful activation of dendritic cells.

In spite of marginal success with lysates as T-cell activators in the past, the present invention continues to rely on use of an autologous tumor lysate as a vaccine antigen. Said strategy is expected to be effective against tumor recurrence because the tumor lysate should contain all the relevant epitopes that can stimulate CD4+ helper T cells (adaptive immune response) and CD8+ T cells (innate immune response). The problem appears to be ineffective programing of dendritic cells, or ineffective activation of T-cells against the antigen by Antigen Presenting Cells (APCs). The present invention overcomes these limitations by targeting the antigen presenting steps to dendritic cells in Peyer's Patches. The present invention further ensures the activation of said dendritic cells by use of adjuvants, and by encapsulation of antigens and adjuvants in cholestosomes so that they will be more readily picked up by dendritic cells in the Peyer's Patches. The invention overcomes the ineffective activation of dendritic cells by peripheral injection of tumor lysates in the past.

By its very nature as a process beginning with an autologous tumor, the use of these antigens in cholestosomes and targeted to Peyer's Patches would be less likely to trigger autoimmune activation of T-cells against non-cancerous tissues and organs of the host. Likewise, there should be no attack from the patient's immune system on the newly activated T-cells, so the use of immunosuppressives as in CAR-T procedures should also be rendered moot. Both problems of current T-cell activation methods are avoided in the present invention.

Unlike previous use of subcutaneous injections of antigens as immune activators in cancer, the present invention relies on oral administration. However, it is necessary to avoid degradation in the anterior GI tract in order to reach the site of immune system programming in the ileum. Thus, antigens are formulated in the outer capsule of the present invention so that there is intact delivery of antigen and optionally adjuvant to the ileum and activation in Peyer's patches. The inventive step of oral use avoids the need for ex-vivo antigenic programing of dendritic cells, and certainly avoids the need to create a suitable mass of activated T-cells ex vivo. In essence, the inventors are, for the first time, programming T-cells against the tumor using a composition from said patients tumor itself, and all activation and expansion steps are conducted in-vivo after they are verified to be active by cytokine expression in vitro.

The necessary amount of tumor received should be at least one gram, and preferably 5 grams or more. Once received in the inventor's laboratory, fresh tumors are minced, washed with PBS to remove blood, and then digested with collagenase (0.5 mg/ml, Cat#C5138, Sigma-Aldrich) for at least 1 hr at 37S. The pieces are then passed through a 100 mm cell strainer to obtain a single-cell suspension.

Tumor Lysates are prepared from the cell suspensions using Freeze-Thaw cycles, with an optimal number of cycles being 5 to ensure that there are no living cells remaining, in the antigenic mixture. Freeze thaw methods for preparing lysates are well known in the art (28). The antigenic composition will be further separated by centrifugation to remove large particles >10 microns. Adjuvants may be added to the reaction mixture at this point.

The coarse filtered and membrane enriched fractions of tumor lysate will be suspended in PBS in a volume of approximately 1.0 ml, and this mixture will be encapsulated into cholestosomes as disclosed previously by the inventors(13, 29).

The cholestosome encapsulated fractions will then he lyophilized and the lyophilized material will be placed into the capsule delivery for ileum targeting. These capsules will be given orally back to the patient from whom the tumor was excised, targeting delivery of said compositions to the ileum and/or the ascending colon in the vicinity of the appendix.

In order to overcome the degradation of antigen in the GI tract, said invention will encapsulate said tumor lysate antigens in a lipid vesicle. When the formulation releases the antigenic, composition at the ileum and the encapsulated vesicles reach the Peyer's Patches, the antigen loaded vesicles preferentially enter the dendritic cells because this property is conveyed by encapsulation in cholestosomes. There is no endosome formation around a cholestosome composition entering dendritic cells, so when the outer surface of the cholestosome is removed by cholesteryl hydrolases in the cytoplasm., the antigen and adjuvant are free in the cytoplasm to allow dendritic cell recognition and packaging to present to T-cells during their activation.

For the first time, this procedure would allow dendritic cell activation in the ileum using a composition that would either activate dendritic cells or in some cases harmlessly pass thru the intestine unabsorbed. In addition to the safety advantage of oral administration of tumor lysate compositions, the encapsulation of said composition in the vesicles ensures uptake of the antigen and adjuvant by only the dendritic cells which are charged with programming activated T-cells against the patient's tumor.

Furthermore, the manufacture of oral dosage forms would be less demanding and certainly more pleasing to the patient than the current manufacture of injectable forms. Alternative delivery of cholestosome encapsulated constructs, such as direct: injection into tumors, can also be applied if the response to oral use is not sufficient to eradicate said patient's tumor.

The preparation method of tumor antigen particles is not particularly limited, and applicable methods include, for example, a method of grinding the solidified tumor tissues to prepare microparticles of fine fragments, as well as a method of lysing ground fragments of tumor tissues or tumor cells to fix the lysate to solid microparticles, a method of fixing soluble tumor antigens such as antigenic peptides and antigenic proteins to solid microparticles and the like. As the solid microparticles, for example, iron powder, carbon powder, polystyrene beads and the like from about 0.05 to 1,000 mm diameter can be used. Usable microparticles include ground tissue fragments, tumor cells or soluble tumor antigens bound to lipid particles such as liposomes so as to be recognized as microparticles by the antigen-presenting cells to allow phagocytosis, or a microparticles obtained by binding soluble tumor antigens, per se, to each other by using a binder or a crosslinking agent.

Sizes of cholestosome vesicles containing a mixture of tumor components are not especially limiting, although a size that allows easy passage into cells by receptor mediated endocytosis in vivo is desirable. It is not necessary to grind fixed tumor cells that are originally in a state of small single cells. However, it is desirable to apply grinding or dispersing treatment when the cells aggregate during the fixation operation. For the grinding or dispersing treatment, treatment with a homogenizer ultrasonic treatment, partial digestion with a digestive enzyme and the like can be used. The microparticles can also be prepared by passing through a screen having a pore size of not more than 1,000 micrometers, preferably not more than 380 micrometers. The preparation of these microparticles is well known to those skilled in the art and the skilled artisan can prepare the microparticles by a single appropriate method or a combination of plural methods.

As a method to prepare the lysate from solidified tumor materials, for example, a method using a proteolytic enzyme can be applied. An example of the proteolytic enzyme includes proteinase K. A method employing an appropriate combination of an enzyme other than the proteolytic enzyme, an acid, an alkali and the like may also be utilized. Any method that can achieve lysis of the solidified tumor material may be employed, and those skilled in the art can choose an appropriate method. The lysate may be fixed to the solid microparticles mentioned above.

### Oral Ileal Delivery of Cholestosome Encapsulated Immunotherapeutics

Said oral delivery of ileal targeted cholestosome encapsulated immunotherapy constructs and adjuvants such as cholestosome encapsulated LPS, will be accomplished using an enterically coated ileal targeted capsule previously disclosed h the inventors as a means of delivery for cancer vaccines. The oral delivery capsule disclosed is specifically coated to release its outer contents at the ileum and its inner contents in the right colon at the site of the appendix(30). The entire disclosure is incorporated herein for reference, since the application of this delivery to cholestosome encapsulated constructs has not previously been attempted.

In the specific practice of the invention, the oral delivery capsules containing cholestosome encapsulated fractions and LPS or other adjuvants will be targeted to release these fractions at the ileum which is the site of Peyer's Patches, a concentrated lymphoid tissue with many dendritic T-cells responsible for surveillance and mucosal surface immune defense. The ileum is the site of T-cell immunity mediated by dendritic cells. Optionally, the inner capsule will be released at the appendix in the right colon, for stimulation of B-cell immunity.

Combination Treatment with Checkpoint Inhibitors

Patients who receive these oral cholestosome fraction treatments for an identical placebo consisting of the capsules with tiller only), will be optionally given PD-1 monoclonal antibodies (for example, nivolumab, pembrolizumab), PD-L1 monoclonal antibodies (for example atezolizumab), and optionally CTLA-4 antibodies for example, Ipilimumab) Patients may also optionally receive cytokine stimulation with IL-2 or general immune system activators such as interferon. One or more of these latter treatments Will be given parenterally according to the instructions of their respective manufacturers. Nonspecific immune stimulation with interleukin 2 (IL-2) and ipilimumah can lead to durable cancer regression, although the overall tumor response rates for each agent have been small (16% for high-dose IL-2 and 11% for ipilimumab), with complete response (CR) rates of less than 10%. A pilot trial of 36 patients with melanoma treated with ipilimumab combined with high- dose IL-2 had overall response (OR) rates of 25%, with 17% achieving CRs lasting more than 8 years ongoing; however, this IL-2 plus ipiliniunab combination has not been further tested to confirm these results. Anti-PD1 and anti-PD-L1 antibodies have been recently reported to have OR rates of up to 38% and 17%, respectively, in patients with melanoma, and OR rates of up to 40% when combined with ipilimumab although the long-tem durability of the responses is not yet known.

Although Immunotherapy with tumor lysate-loaded dendritic cells (DCs) is one of the most promising strategies to induce antitumor immune responses, the antitumor activity of cytotoxic T cells may be restrained by their expression of the inhibitory T-cell coreceptor cytotoxic lymphocyte antigen-4 (CTL-A-4), which is a checkpoint inhibitor pathway. By relieving this restraint, CTIA-4-blocking antibodies promote tumor rejection, but the full scope of their most suitable applications has yet to be fully determined.

Others have studied the checkpoint inhibitors in conjunction with minor lysates. A preclinical concept in a C3H mouse osteosarcoma (LM8) model that CTLA-4 blockade cooperates with cryofreated tumor lysate-pulsed DCs in a primary tumor to prevent the outgrowth of lung metastasis. To evaluate immune response activation, the authors established the following four groups of C3H mice (60 mice in total): i) control immunoglobulin G (IgG)-treated mice; ii) tumor lysate-pulsed DC-treated mice; iii) anti-CTLA-4 antibody-treated mice and iv) tumor lysate-pulsed DC- and anti-CTLA-4 antibody-treated mice. The mice that received the tumor lysate-pulsed DCs and anti-CTLA-4 antibody displayed reduced numbers of regulatory T lymphocytes and increased numbers of CD8+ T lymphocytes inside the metastatic tumor, inhibition of metastatic growth, a prolonged lifetime, reduced numbers of regulatory T lymphocytes in the spleen and high serum interferon-gamma levels. Combining an anti-CTLA4 antibody with tumor lysate-pulsed DCs enhanced the systemic immune response. These findings document for the first time an effect of the combination of tumor lysate-pulsed DCs and CTLA-4-blocking antibodies in osteosarcoma. The authors suggest that cryotreated tumor lysate-pulsed DCs, although insufficient on their own, may mediate the rejection of metastatic lesions and prevent recurrence of the disease when combined with CTLA-4 blockade in osteosarcoma patients in the clinical setting.(31)

In view of these promising results with tumor lysates, checkpoint inhibitors may be both combined with lysates as separate treatments, or alternatively, checkpoint inhibitors may be added to cholestosomes as a means of reaching receptors on Dendritic cells, T cells or on cancer cells directly. These compounds are usually monoclonal antibodies, and the examples of monoclonal antibodies subjected to cholestosome encapsulation presented herein show good encapsulation of similar size molecules, as well as intracellular delivery in cell models.

Accordingly, one preferred aspect of the present invention is to include both tumor antigens and checkpoint inhibitors along with an adjuvant in the oral delivery capsule of the present invention.

In a further embodiment designed to overcome high level PD-1 expression by the tumor, the antigenic mixture encapsulated in the cholestosomes would contain siRNA oligonucleotides specific for PD-L1 and PD-L2. siRNA-mediated knockdown of PD-1 ligands on human T cells augments the function of these cells, including 11⁷N-g production and CTL activity. Accordingly, the inventors will incorporate cholestosome encapsulated anti PD-1 oligos of siRNA into the compositions delivered to Peyer's Patches, in order to reduce the cell surface and mRNA expression of the target PD-1 expression proteins on the surface of the activated T cells. The reductions of PD-1 ligands by siRNA oligos would be confirmed by real time reverse transcriptase-PCR. When said siRNAs are added to compositions, supernatants examined for the presence of interferon-g (IFN-g), and siRNA associated knockdown of either PD-L1, I or PD-L2 expression by T-cell clones would reveal an increase in IFN-g production. Furthermore, an siRNA-transfected CD8+ T-cell clone would exhibit increased lytic activity toward in vitro lysate antigens.

In addition to PD-1 blockers, it is an alternative embodiment to incorporate cell excitatory cytokines in the cholestosome encapsulated mixture, in order to further activated dendritic cells against the tumor antigens. IL-2 and IL-12 are suitable for this purpose, as both cytokines have proven to excite T-cells against tumors in routine use. Ileal delivery of cholestosome encapsulated compositions to include tumor lysates, PD-1 antibodies and cytokines such as IL-12 are within the scope of the invention.

A further embodiment is to include a stimulating substance that activates antigen-presenting cells such as dendritic cells and improves T-cell priming. Such a substance would be a TLR9 agonist, recently disclosed IMO-2125 or the like, Currently IMO-2125 is being injected into the tumor microenvironment, and thus given in combination therapy with checkpoint inhibitors such as ipilimumab or pembrolizumab. In preclinical studies, intratumor injected IMO-2125 stimulated plasmacytoid dendritic cells to induce high amounts of interferon alpha and helper T cell-i cytokines, leading to increased immune cell infiltration in the tumor microenvironment. In addition, the combination of intratumor IMO- 2125 with either an anti-CTLA-4 or anti-PD-1 antibody resulted in improved systemic tumor control compared with either agent alone. Use of cholestosome encapsulated IMO -2125 would be expected to strongly stimulate dendritic cells, and thereby improve the activation step when this substance is injected into tumors, it is within the scope of the invention to use cholestosome encapsulated IMO-2125 as delivered to the dendritic cells in the ileum. The cholestosome encapsulated EMO-2125 would be administered in combination with either systemically injected checkpoint inhibitors in effective amounts, or alternatively in combination with orally administered cholestosome encapsulated checkpoint inhibitors in order to boost the responsiveness of tumors to checkpoint inhibitors.

All patients receiving said immunotherapy will be monitored for in vivo tumor responsiveness and for activation of CD4+ and CD8+ T-cells, using flow cytometric methods. Tumor lysis/regression will be documented by RECIST and mechanistically by repeat biopsy to show T-cell infiltration of the tumor as well as lysis and regression.

### Flow Cytometric Methods.

MCF-7 cells were plated at 750,000 cells per well on 12-well plates one day prior to treatment. Next day, media was removed and plates were washed twice with warm (37° C.) Ringer buffer (pH 7.4). Cells were serum starved for 1.0 hour in supplemented, serum-free DMEM, then 2,3 ug/mL FITC-Cholestosomes were added in triplicate to wells containing serum-free DMEM. Three wells were untreated to serve as unstained controls. Plates were incubated at 37° C. for indicated times in FIG. 1. Following treatment, wells were washed 4 times with ice-cold Ringer's. Cells were prepared for flow cytometry as described before. DRAQ5 (nuclear stain) was not used in these studies as we previously found that >90% of events in our defined single-cell gate stained positive for DRAQ5.

For the 4.5 hr +FBS, cells were handled as described above: wells were washed and cells starved 1 hour prior to treatment. Cells were allowed to incubate with FITC-Cholestosomes for 4.5 hours, then FBS was spiked into the wells for a final concentration of 10% v/v. The total volume within these wells increased by 50 uL, diluting what, would have been the initial FITC-Cholestosome concentration to 2.1 ug/mL. Cells were allowed to incubate with FITC-Cholestosomes for a total of 24 hours, then prepared for flow cytometry.

FITC-Cholestosomes demonstrated a time-dependent accumulation within MCF-7 cells, with measurable uptake detected at 30 minutes prior to treatment. We were unable to achieve steady state kinetics, which may be due to the concentration used for these studies. Notably, the cells that received a spike of FBS 4.5 hours after addition of FITC-Cholestosomes demonstrated significant vesicle uptake. The mean fluorescence intensity was significantly lower than serum-starved cells and may be due to (1) sample dilution (from FBS addition) as well as (2) signal dilution from cell growth (serum-starved cells are growth arrested). We provide the first Flow Cytometric data to support time-dependent uptake of FITC-Cholestosomes is MCF-7 cells at a FITC concentration of 2.3 ug/mL. These results will be confirmed using either flourescent microscopy and/or measuring intracellular FITC concentrations on a fluorimeter following cell lysis.

### INDUSTRIAL APPLICABILITY

As has been described above, the compositions and methods described herein make it possible to provide an excellent and well characterized antigenic and optionally adjuvanted composition for augmenting the killing of a specific tumor in a specific patient. Some of the generally applicable antigens may allow the practice of the invention without sampling of the tumor itself, but these are applications and not necessarily the preferred embodiments herein. The disclosed personalized oral immunotherapy is ready for use in patients, but does require approval by regulatory authorities prior to commercialization.

### REFERENCES

Kidron M. Patent: Methods and Compositions for Oral administration of Exenatide, US 2013-0195939 A1. 2013; Published Aug. 1, 2013.
2. Irvine D J, Moon J. Patent: Lipid Vesicle Compositions and Methods of Use. application Ser. No. 13/052,067. 2011; Provisional U.S. 61/315,485 tiled Mar. 19, 2010.
3. Balu-Iyer S, Banken R B, Purohit V S. Patent: Compositions and methods of preparation of Liposomal Microparticulate IL-12. U.S. 60/706,650 filed Aug. 9, 2005. 2010; U.S. Pat. No. 7,662,405 issued
4. Zhu G, Mallery S R, Schwendeman S P. Stabilization of proteins encapsulated in injectable poly (latide-co-glyeolide). Nat Biotechnol. 2000; 18(1): 52-7.
5. Tseng L-P, Liang H-J, Chung T-W, Huang Y-V, Liu D-Z. Liposomes Incorporated with Cholesterol for Drug Release. J. Medical and Biological Engineering. 2007; 77:29-34.
6. Geho W B, Lau J. Patent; Orally Bioavailable Lipid based constructs. US 2013-0183270 A1. 2013; Application U.S. Ser. No. 13/785,591 Filed Mar. 5, 2013.
7. Sonaje K, Lin K J, Wey S P, Lin C K, Yeh T M, Nguyen H N, et al. Biodistribution, pharmacodynamics and pharmacokinetics of insulin analogues in a rat model: Oral delivery using pH-responsive nanoparticles vs. subcutaneous injection. Biomaterials. 2010; 31(26):6849-58.
8. Su F Y, Lin K J, Sonaje K, Wey S P, Yen T C, Ho Y C, et al. Protease inhibition and absorption enhancement by functional nanoparticles for effective oral insulin delivery. Biomaterials. 2012; 33(9):2801-11.
9. Sung H W, Sonaje K, Liao Z X, Hsu L W, Chuang E Y. pH-responsive nanoparticles shelled with chitosan for oral delivery of insulin: from mechanism to therapeutic applications. Acc Chem Res. 2012; 45(4):619-29.
10. Hong K, Zheng W-W, Drummond D, Kirpotin D, Hayes M. Patent; Delivery of Nucleic Acid-Like Compounds. Filed May 15, 2002. As U.S. 60/381,417. 2004,
11. Nagy J O, Federman N, Denny C, Tomlinson J S. Patent: Enhanced Growth :Inhibition of Osteosarcoma by Cytotoic Polymerized Liposomal Nanoparticles Targeting the Alcam Cell Surface Receptor application Ser. No. 14/116,688, 2014; Provisional application No. 61/485,024 filed on May 11, 2011.
12. McCourt M P, Patent: Drug Delivery Means. U.S. Provisional application 60/784,118 Filed Mar. 20, 2006, 2006; U.S. Non-Provisional application Ser. No. 11/725,831 Filed Mar. 20, 2007(Published on Sep. 27, 2007 as US2007-0225264 A1):Issued as U.S. Pat. No. 9,119,787 on Feb. 5, 2015.
13. Schentagn, J J, McCourt M P, Mielnicki L, Hughes J. Patent: Cholestosome Vesicles for Incorporation of Molecules into Chylomicrons. U.S. Provisional application 61/783,003 Filed Mar. 14, 2013; PCTUS2014/027761 filed Mar. 14, 2014; U.S. Nonprovisional application Ser. No. 14/776,308 filed Mar. 13, 2014; (Published on Sep. 25, 2014 as WO2014-152795 and Feb. 4, 2016 as US 2016-0030361):Status: Issued Jul. 4, 2017 as U.S. Pat. No. 9,693,968.
14. Abumrad N, Harmon C, Ibrahimi A. Membrane transport of long-chain fatty acids: evidence for a facilitated process. J. Lipid Res. 1998; 39(12):2309-18.
15. Trotter P J, Ho S Y, Storch J, Fatty acid uptake by Caco-2 human intestinal cells. J Lipid Res. 1996; 37(2):336-46.
16. Negrete G R, Mahindrarantne M, Mfuh A M, Quintero M V, Patent: Compositions and. Methods related to Acid Stable Lipid Nanospheres, US 2011-0268653 Published Nov. 3, 2011, 2011; Provisional 61/325,111 filed Apr. 16, 2010.
17. Leissring M A, Malito E, Hedouin S, Reinstatler L, Sahara T, Abdul-Hay S O, et al. Designed inhibitors of insulin-degrading enzyme regulate the catabolism and activity of insulin, PLoS One, 2010; 5(5):e10504.
18. Bannister T D, Wang H, Abdul-Hay S O, Masson A, Madoux F, Ferguson J, et al. ML345, A Small-Molecule Inhibitor of the Insulin-Degrading Enzyme (IDE). Probe Reports from the NIH Molecular Libraries Program. Bethesda (Md.); 2010.
19. Delgado A, Lavelle E C, Hartshorne M, Davis S S. PLG microparticles stabilised using enteric coating polymers as oral vaccine delivery systems. Vaccine. 1999; 17(22)2927-38.
20. Ano G, Esquisabel A, Pastor M, Talavera A, Cedre B, Fernandez S, et al. A new oral vaccine candidate based on the microencapsulation by spray-drying of inactivated Vibrio cholerae. Vaccine. 2011; 29(34):5758-64.
21. Luchoomun J, Hussain M M. Assembly and secretion of chylomicrons by differentiated Caco-2 cells. Nascent triglycerides and preformed phospholipids are preferentially used for lipoprotein assembly. J Biol Chem. 1999; 274(28):19565-72.
22. Himoudi N, Abraham J D, Fournillier A, Lone Y C, Joubert A, Op De Beeck A, et al. Comparative vaccine studies in HLA-A2.1-transgenic mice reveal a clustered organization of epitopes presented in hepatitis C virus natural infection. J Virol. 2002; 76(24):12735-46.
23. Cheng L, Fu J, Tsukamoto A, Hawley R G. Use of green fluorescent protein variants to monitor gene transfer and expression in mammalian cells. Nat Biotechnol. 6; 14(5 ):606-9.
24. Mandian R, Kokhaei P, Najar H M, Derkow K, Choudhury A, Mellstedt H. Dendritic cells, pulsed with lysate of allogeneic tumor cells, are capable of stimulating MHC-restricted antigen-specific antitumor cells. Med Oncol. 2006; 23(2):273-82.
25. Su S, Flu B, Shao J, Shen B, Du J, Du Y, et al, CRISPR-Cas9 mediated efficient PD-1 disruption on human primary T cells from cancer patients. Sci Rep. 2016; 6:20070.
26. Ichim T, Tullis R H. Patent: Extracoporeal Removal of Microvesicular Particles. US 2014-0166578 as published Jun. 19, 2015. 2014 application Ser. No. 14/185,033 Filed Feb. 20, 2014(Provisional Application 60/780,945 filed Mar. 9, 2006):Divisional of U.S. Pat. No. 8,288,172.
27. Kreiter S, Vormehr van de Roemer N, Diken M, Lower M, Diekmann J. et al. Mutant MHC class II epitopes drive therapeutic immune responses to cancer. Nature. 2015; 520(7549):692-6.
28. Kawahara M, Takaku H. A tumor lysate is an effective vaccine antigen for the stimulation of CD4(+) T-cell function and subsequent induction of antitumor immunity mediated by CD8(+) T cells. Cancer Biol Ther. 2015; 16(11):1616-25.
29. Schentag J J, McCourt M P, Mielnicki L, Patent: Chylomicrons as Carriers for Cholesteryl Ester Vesicles Loaded with Peptides and Proteins for Oral Absorption and Intracellular Delivery. US Provisional Application 62/378,599 Filed Aug. 23, 2016:Status: Active Negotiation.
30. Schemag J J, Kabadi M. Patent: Gastrointestinal Site-Specific Oral Vaccination Formulations Active on the ileum and Appendix. US Provisional application 61/617,367 Filed Mar. 29, 2012; application Ser. No. 14/387,979 Filed on Mar. 14, 2013; Filed as PCT/US2013-031483 and PCT/U.S. Ser. No. 13/31,483 filed on Mar. 13, 2013(Published on Mar. 12, 2015 as US2015-0071994 A 1).
31. Kawano M, ltonaga I, Iwasaki T, Tsumura H. Enhancement of antitumor immunity by combining anti-cytotoxic T lymphocyte antigen-4 antibodies and cryotreated tumor lysate-pulsed dendritic cells in murine osteosarcoma. Oncol Rep. 2013; 29(3):1001-6.

1. A composition in pharmaceutical dosage form for administration to a patient or subject comprising one or more macromolecules encapsulated in a lipid vesicle to provide an intact loaded vesicle wherein the outer surface coating of said loaded vesicle comprises at least one cholesteryl ester obtained from cholesterol and a C₈-C₂₆ fatty acid, said macromolecule obtaining an intracellular concentration in cells of said patient or subject which is at least 10-fold greater than the concentration obtained by said macromolecule in the absence of said vesicle.

2. The composition according to claim 1 wherein said outer surface coating of said loaded vesicle remains intact without endosomal formation during passage of said loaded vesicle across a membrane of a cell in said patient or subject, and wherein said vesicle optionally (I don't think we need this word, but I am OK with keeping it too; releases said one or more macromolecules inside the cell by the action of cholesteryl ester hydrolases on said vesicle.

3. The composition according to claim 1, wherein said macromolecule is selected from the group consisting of proteins, peptides, nucleic acids and mixtures thereof.

4. The composition according to claim 1 wherein said macromolecule when encapsulated in said vesicle achieves an intracellular concentration at least 250-fold greater than the concentration achieved by said macromolecule in the absence of said vesicle.

5. The composition according to claim 1 wherein said intact vesicle is taken into cells of said patient or subject which express surface receptors for said chylomicrons and whereby once inside said cells, said intact vesicle releases said macromolecule(s) in said cell by the action of cholesteryl ester hydrolases in said cells on said vesicle.

6. The composition according to claim 5 wherein the intracellular concentration of macromolecule in cells expressing surface chylomicrons receptors is at least 10 times greater than the intracellular concentration of macromolecule in cells which do not express a surface chylomicrons in said patient or subject.
Note to Henry. This #7 does not add anything to 5 and 6, so I deleted it. Keeping it would need a re-write

7. The composition according to claim 2 wherein the intracellular concentration of said intact vesicle containing said macromolecule in cells expressing a surface chylomicrons receptor is at least 250 times greater than the intracellular concentration of said intact vesicle containing said macromolecule in cells which do not express said surface chylomicrons receptors.

8. (canceled)

9. (canceled)

10. The composition according to claim 1 wherein said cell metabolizes said macromolecule and/or ejects said macromolecule from said cell. ?? Reverse the order . . . what is now claim 11 should be 10, and what is now 10 should be 11??

11. The composition according to claim 1 wherein said macromolecule is unaltered during encapsulation into said intact vesicle and upon release in said cells by cholesteryl ester hydrolase and is identical to and has the same activity as said macromolecule encapsulated in said vesicle.

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. The composition according to claim 3 wherein oral administration of said macromolecule in said vesicle produces bioavailability of said macromolecule in said patient or subject of at least 50% in comparison to said macromolecule not encapsulated in said vesicle.

36. The composition according to claim 35 wherein said bioavailability is 85-100%.

37. The composition according to claim 3 wherein oral administration of said macromolecule produces a tissue concentration at least 10 times greater than the plasma concentration of said agent.

38. The composition according to claim 3 wherein oral administration of said macromolecule produces a tissue concentration at least 20 times greater than the plasma concentration of said agent.

39. The composition according to claim 3 wherein oral administration of said macromolecule produces a tissue concentration up to 250 times the plasma concentration of said agent.

40. The composition according to claim 1 wherein said cholesteryl esters are a mixture of two different cholesteryl esters.

41. The composition according to claim 41 wherein said cholesteryl ester components are obtained from fatty acids which differ in length by more than two carbon units.

42. The composition according to claims 41 wherein said cholesteryl ester components are obtained from fatty acids which differ in length by no more than two carbon units.

43. The composition according to claim 41 wherein said cholesteryl ester components are obtained from fatty acids which differ in length by two carbon units.

44. The composition according to claim 41 wherein said vesicles further comprise an effective amount of phosphatidyl serine to target cells for apoptosis.

45. The composition according to claim 3 wherein said macromolecule is a nucleic acid or includes a nucleic acid.

46. The composition according to claim 46 where said nucleic acid is pgWizGFP plasmid.

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. The composition according to claim 1 wherein said fatty acid is selected from the group consisting of Myristoleic acid, Palmitoleic acid, Sapienic acid, Oleic acid, Elaidic acid, Vaccenic acid, Linoleic acid, Linoelaidic acid, α-Linolenic acid, Arachidonic acid, Eicosapentaenoic acid, Erucic acid, Docosahexaenoic acid, Caprylic acid, Capric acid, Lauric acid, Myristic acid, Palmitic acid, Stearic acid, Arachidic acid, Behenic acid, Lignoceric acid, Cerotic acid or a mixture thereof.

59. (canceled)

60. (canceled)

61. (canceled)

62. (canceled)

63. (canceled)

64. A composition comprising a tumor lysate or a tumor antigen encapsulated in a lipid vesicle wherein the outer surface coating of said vesicle is comprised of one or more cholesteryl esters obtained from cholesterol and a C₆-C₂₆ fatty acid (preferably a C₈-C₂₂ fatty acid, or a C₈-C₁₄ fatty acid), wherein said outer surface coating of said vesicle remains intact during passage of said composition across a cell membrane such that said vesicle and its core enters into lymphoid cells, including dendritic cells without endosomal formation, and releases said encapsulated lysate or antigen in said cells by the action of cellular cholesteryl ester hydrolases, wherein the intracellular concentration of said lysate or antigen in said cell is at least 10 fold and preferably at least 250 fold greater than the intracellular concentration that would be obtained from the same concentration of tumor lysate or antigen being delivered into said cells if it were not encapsulated in said vesicle.

65. The composition according to claim 64 wherein said tumor lysate or antigen additionally comprises macromolecules which are selected from the group consisting of proteins, peptides, nucleic acids or mixtures thereof.

66. (canceled)

67. (canceled)

68. (canceled)

69. (canceled)

70. (canceled)

71. (canceled)

72. (canceled)

73. (canceled)

74. (canceled)

75. (canceled)

76. (canceled)

77. (canceled)

78. A method of treating cancer in a patient in need comprising orally administering a composition according to claims 64-65 to said patient, wherein said composition activates a cellular immune response against cancer cells in said patient.

79.-82. (canceled)

83. The composition according to claim 2 in oral dosage form for administration to a patient or subject wherein said loaded vesicles enter the duodenal enterocytes in an intact form and remain intact while said enterocyte incorporates said loaded vesicles into chylomicrons for release into lymphatic fluids and eventual delivery into cells of said patient or subject.

84. The composition according to claim 1 in oral dosage form which is optionally enteric coated, wherein said macromolecule reaches the blood stream of said patient or subject at a concentration between at least 50 percent and 100 percent of the area under the curve (AUC) blood concentration when said macromolecule is administered to said patient or subject by subcutaneous or intravenous injection.

85. The composition according to claim 1 wherein oral administration of said intact vesicle causes said vesicles to concentrate in cells of said patient or subject which express surface receptors for said chylomicrons and following said passage, said intact vesicle releases said macromolecule(s) in said cell by the action of cholesteryl ester hydrolases in said cells on said vesicle.

86. The composition according to claim 5 wherein the oral administration of said vesicle results in the intracellular concentration of macromolecule in cells expressing surface chylomicron receptors that is at least 10 times greater than the intracellular concentration of said macromolecule in cells which do not express a surface chylomicron receptor.
